(19) United States
(12) Patent Application Publication
(10) Pub. No.: US 2026/0325099 A1
(43) Pub. Date: Oct. 22, 2026

(54) DEVICES, SYSTEMS, AND METHODS FOR MONITORING AND MODULATING THE NERVOUS SYSTEM USING CONTRAST-ENHANCED ULTRASOUND-BASED BRAIN-COMPUTER INTERFACE

(71) Applicant: Forest Neurotech, LLC, Cambridge, MA (US)

(72) Inventors: Sumner Lee NORMAN, Los Angeles, CA (US); Tyson Nikiyas Sunyata AFLALO, Claremont, CA (US)

(21) Appl. No.: 19/651,419

(22) Filed: Apr. 17, 2026

Related U.S. Application Data
(60) Provisional application No. 63/791,308, filed on Apr. 18, 2025.

(51) Int. Cl. A61B 8/08; A61B 8/00

(52) U.S. Cl. CPC A61B 8/0808; A61B 8/481

(57) ABSTRACT

Devices, methods, and systems related to ultrasound imaging or modulating a nervous system for a subject comprising a sono-enhancing contrast agent are described. The ultrasound imaging or modulating of the nervous system can include the use of one or more ultrasound transducers, wherein the one or more ultrasound transducers can be optimized for a specific scale, e.g., a brain-wide scale, a brain region-level scale, a neural circuit-level scale, or a single neuron-level scale. The methods and systems may also comprise, for example, a method of imaging or modulating the nervous system of a subject comprising a sono-enhancing contrast agent, based on a closed-loop operation. The closed-loop operation can be based on a predictive closed loop, or a non-predictive (e.g., reactive) closed loop.

### CROSS-REFERENCE TO RELATED APPLICATIONS

This application claims the priority benefits of U.S. Provisional Patent Application No. 63/791,308, filed Apr. 18, 2025, the contents of which are incorporated herein by reference in its entirety.

### FIELD OF THE DISCLOSURE

The present disclosure relates generally to devices, systems, and methods for monitoring or modulating one or more portions of a nervous system of a subject using one or more ultrasound transducers and a sono-enhancing contrast agent. More specifically, the parameters of the devices, systems, and methods can range to accommodate for the optimal monitoring or modulating of a whole brain, one or more brain regions, one or more neural circuits, or one or more single neurons.

### BACKGROUND

Existing methodologies for monitoring or modulating the nervous system are constrained to specific spatial domains. A given methodology often falters when transitioning between whole-brain, regional, circuit, or cellular analyses. Clinical applications and research, however, benefit from the analysis of diverse biological scales. Currently, a motley assortment of technological modalities are used for studying neural phenomena across scales. The use of such assortments result in added instrumentation expenses, high maintenance costs, and non-cohesive data structures. The devices, systems, and methods described herein address these challenges.

### BRIEF SUMMARY

Dysfunctions across multiple scales of the nervous system result in neural disorders. Existing neurotechnology fails to provide a unified and scalable platform for monitoring or modulating the nervous system. As a result, clinicians and researchers are limited to using entirely different technological modalities when monitoring or modulating the nervous system across its different scales. For example, the monitoring or modulating of the nervous system at the level of the whole brain often requires different technological modalities from those used to study specific brain regions, specific neural circuits, or individual neurons. This lack of scalability increases procedural complexity, raises costs, and often necessitates multiple invasive interventions for subjects. In addition, the lack of a unified platform creates substantial data integration challenges for clinicians and researchers.

The present disclosure addresses these challenges by providing a unified technological platform that works across all neural scales, from whole brain to single neurons. The devices, systems, and methods disclosed herein employ specialized ultrasound transducers that share common operational principles and data processing frameworks. Each transducer is optimized for a specific neural scale: larger element ultrasonic transducer arrays (e.g., 100-500 microns) and lower frequencies (1.0-2.5 MHz) for whole-brain applications, mid-sized element ultrasonic transducer arrays (e.g., 50-250 microns) and mid-range frequencies (5.0-15 MHz) for brain regions and neural circuits, and small element ultrasonic transducer arrays (e.g., 20-50 microns) and high-range frequencies (e.g., 15-100 MHz) for neural circuits and single neurons.

In addition to employing scale-optimized transducer elements, the ultrasound transducers further optimize for their target neural scale be emitting scale-specific ultrasound frequencies—high penetrating lower frequencies (e.g., 2.5-5.0 MHz) are used for low-resolution whole-brain monitoring or modulating, intermediate frequencies (e.g., 5.0-15 MHz) are used for regional monitoring or modulating, and low penetrating high frequencies (e.g., 15-100 MHz) are used for high-resolution circuit and cellular monitoring. Despite the scale-optimization of the ultrasound transducers, the ultrasound transducers disclosed herein employ a unifying technological design. They interface with the same control systems and generate neural data structures in compatible formats.

One challenge facing ultrasound and other volumetric imaging techniques is that at smaller imaging volumes and higher imaging resolutions, the signal-to-noise ratio supralinearly decreases. Such a supralinear decrease can result in irrecoverable imaging signals or non-effective neuro-modulations, when applying the ultrasound transducers optimized for emitting and receiving intermediate frequencies or high frequencies. The devices, systems, and methods disclosed herein, address the signal-to-noise challenge, via the use of sono-enhancing contrast agents. Sono-enhancing contrast agents can, upon being activated by the ultrasound waves emitted by the ultrasound transducer, emit strongly scattered ultrasound waves that are readily received and signal-processed by the ultrasound transducer. These contrast agents allow for the maintenance of high-quality neural monitoring or modulating, at even the finest spatial resolutions or the deepest tissue penetrations. The sono-enhancing contrast agents can be biological, and can include genetically encoded acoustic proteins, such as ultrasonic reporters of calcium, ultrasonic reporters of voltage, or ultrasonic reporters of a neurotransmitter, e.g., glutamate, GABA, dopamine, or acetylcholine.

The combination of scale-optimized ultrasound transducers and sono-enhancing contrast agents constitute a unified and reliable platform for monitoring or modulating a nervous system. The devices, systems, and methods disclosed herein allow for a scalable and dependable ultrasound-based neurotechnology platform that can monitor or modulate the nervous system at a brain-wide, brain region-specific, neural circuit-specific, or neuronal-specific scale. By providing a unified technological platform with scale-optimized transducers for each neural scale, the devices, systems, and methods disclosed herein reduce procedural complexities, decreases costs, and reduces invasiveness, when compared to the existing standard of using several disparate technologies.

In some aspects, disclosed herein is an ultrasound transducer configured to emit ultrasound waves, or receive the ultrasound waves, from one or more portions of a nervous system from a subject comprising a sono-enhancing contrast agent, the ultrasound transducer comprising: a sonolucent window; an ultrasonic transducer array, wherein an element in the ultrasonic transducer array can be 20 to 500 microns wide, and the ultrasonic transducer array can comprise at least 100 elements; a control circuit configured to receive or execute instructions for activating a modulating mode or a monitoring mode for the ultrasound transducer; and a processing circuit configured to transform the received ultrasound waves into one or more neural data structures.

In some aspects, disclosed herein is an ultrasound transducer configured to emit ultrasound waves, or receive the ultrasound waves, from one or more portions of a nervous system from a subject comprising a sono-enhancing contrast agent, the ultrasound transducer comprising: a sonolucent window; an ultrasonic transducer array, wherein an element in the ultrasonic transducer array can be 20 to 500 microns wide, the ultrasonic transducer array can comprise at least 100 elements, and the ultrasonic transducer array can be configured to receive or execute instructions for modulating the one or more portions of the nervous system from the subject; and a processing circuit configured to transform the received ultrasound waves into one or more neural data structures.

In some aspects, disclosed herein is an ultrasound transducer configured to emit ultrasound waves, or receive the ultrasound waves, from one or more portions of a nervous system from a subject comprising a sono-enhancing contrast agent, the ultrasound transducer comprising: a sonolucent window; an ultrasonic transducer array, wherein an element in the ultrasonic transducer array can be 20 to 500 microns wide, the ultrasonic transducer array comprises at least 100 elements, and the ultrasonic transducer array can be configured to receive or execute instructions for monitoring the one or more portions of the nervous system from the subject; and a processing circuit configured to transform the received ultrasound waves into one or more neural data structures.

In any of the embodiments herein, the one or more portions of the nervous system from the subject can comprise a whole brain, a brain region, a neural circuit, or a single neuron. In any of the embodiments herein, the one or more portions of the nervous system from the subject can comprise a dorsolateral prefrontal cortex, frontopolar cortex, anterior cingulate cortex, inferotemporal cortex, visual cortex, auditory cortex, somatosensory cortex, posterior parietal cortex, temporoparietal junction, nucleus accumbens, ventral tegmental area, basal ganglia, orbitofrontal cortex, amygdala, insula, ventromedial prefrontal cortex, hippocampus, entorhinal cortex, parahippcampal gyrus, locus coerulus, reticular formation, ventral striatum, anterior insula, Broca's area, Wernicke's area, medial prefrontal cortex, temporoparietal junction, anterior insula, posterior insula, supplementary motor area, pre-supplementary motor area, or a combination thereof.

In any of the embodiments herein, the one or more neural data structures can correspond to an imaging volume of at most 0.2 cm³. , 0.6 cm³. , 12 cm³. , 100 cm³. , 700 cm³ or 1000 cm³. . In any of the embodiments herein, the one or more neural data structures can comprise imaging data at an optical resolution. In some embodiments, the optical resolution can correspond to approximately a width of the element in the ultrasonic transducer array. In some embodiments, the optical resolution can correspond to approximately the width of the element in the ultrasonic transducer array, based on an approximately one-to-one ratio. In any of the embodiments herein, the optical resolution can be at least 20 microns, 25 microns, 50 microns, 100 microns, 150 microns, 200 microns, 250 microns, 300 microns, or 500 microns. In any of the embodiments herein, the one or more neural data structures can comprise precursor imaging data at a computational superresolution. In some embodiments, the computational superresolution can correspond to less than the width of the element in the ultrasonic transducer array. In any of the embodiments herein, the computational superresolution can correspond to approximately the width of the element in the ultrasonic transducer array, based on an approximately multi-to-one ratio. In any of the embodiments herein, the one or more neural data structures can comprise imaging data at an imaging depth. In some embodiments, the imaging depth can be at least 0.04 cm, 0.1 cm, 0.2 cm, 0.4 cm, 0.5 cm, 2.0 cm, 4.0 cm, 6.0 cm, or 8.0 cm. In any of the embodiments herein, the ultrasound waves can comprise a frequency of at most 100 MHz, 60 MHz, 15 MHz, 7.5 MHz, 5.0 MHz, 2.5 MHz, or 1.0 MHz. In some embodiments, the optical resolution can correspond to approximately the frequency. In any of the embodiments herein, the computational superresolution can correspond to approximately the frequency. In any of the embodiments herein, the one or more neural data structures can comprise hemodynamic data. In any of the embodiments herein, the one or more neural data structures can comprise neural activity data. In any of the embodiments herein, the one or more neural data structures can comprise neural anatomy data.

In any of the embodiments herein, the element in the ultrasonic transducer array can be circular, elliptical, square, rectangular, hexagonal, regular, or irregular, in shape. In any of the embodiments herein, the ultrasonic transducer array can be a capacitive micromachined ultrasonic transducer (CMUT) array or a piezoelectric micromachined ultrasonic transducer (PMUT) array. In some embodiments, the CMUT array can comprise a flat membrane CMUT, a curved membrane CMUT, a dual-electrode CMUT, a collapsed-mode CMUT, a wafer-bonded CMUT, a surface-micromachined CMUT, a CMUT-on-CMOS integrated device, a piston-shaped CMUT, an annular array CMUT, or a flexible CMUT. In some embodiments, the PMUT array can comprise a unimorph PMUT, a bimorph PMUT, a dome-shaped PMUT, a suspended membrane PMUT, an aluminum nitride PMUT, a lead zirconate titanate PMUT, a scandium-doped aluminum nitride PMUT, a lithium niobate PMUT, a polyvinylidene fluoride PMUT, a thin-film PMUT, or a flexible PMUT.

In any of the embodiments herein, the ultrasound transducer can be an implantable ultrasound transducer. In any of the embodiments herein, the ultrasound transducer can be a non-implantable ultrasound transducer. In some embodiments, the non-implantable ultrasound transducer can comprise a wearable ultrasound transducer.

In any of the embodiments herein, the sono-enhancing contrast agent can comprise a biological sono-enhancing contrast agent, a polymeric sono-enhancing contrast agent, or a pharmaceutical sono-enhancing contrast agent. In some embodiments, the biological sono-enhancing contrast agent can comprise a genetically-encoded sono-enhancing contrast agent. In some embodiments, the genetically-encoded sono-enhancing contrast agent can comprise an ultrasonic reporter of calcium, an ultrasonic reporter of voltage, or an ultrasonic reporter of a neurotransmitter, e.g., glutamate, GABA, dopamine, or acetylcholine.

In any of the embodiments herein, the subject can comprise a human subject or a non-human primate subject. In any of the embodiments herein, the subject can have, or can be suspected of having, a neural dysfunction. In some embodiments, the neural dysfunction can comprise a motor dysfunction, a sensory dysfunction, a cognitive dysfunction, a neurological condition, or a neuropsychiatric dysfunction. In some embodiments, the motor dysfunction can comprise a spinal cord injury, amyotrophic lateral sclerosis, locked-in syndrome, stroke, Parkinson's disease, or essential tremor. In any of the embodiments herein, the sensory dysfunction can comprise blindness, deafness, epilepsy, a loss of chemosensation, or a loss of somatosensation. In any of the embodiments herein, the neuropsychiatric dysfunction can comprise clinical depression, schizophrenia, addiction, post-traumatic stress disorder, eating disorder, or sleep disorder.

In any of the embodiments herein, the ultrasound transducer can provide a neural augmenting to the subject. In any of the embodiments herein, the neural augmenting can comprise sensorimotor integrating, sensory augmenting, integrating to a virtual reality, integrating to an augmented reality, modulating a brain state, or integrating to an artificial intelligence.

In some aspects, disclosed herein is a system for monitoring or modulating one or more portions of a nervous system from a subject, comprising: one or more ultrasound transducers, an ultrasound transducer of the one or more ultrasound transducers corresponding to the ultrasound transducer of any of the embodiments herein; a controller coupled to each of the one or more ultrasound transducers, the controller comprising a power source and a processor, wherein the power source is configured to power each of the one or more ultrasound transducers; and wherein the processor is configured to execute a method, the method comprising: sending one or more signals to the one or more ultrasound transducers; and receiving one or more neural data structures from the one or more ultrasound transducers. In some embodiments, the method can further comprise emitting or receiving, via the one or more ultrasound transducers, ultrasound waves.

In any of the embodiments herein, the one or more portions of the nervous system from the subject can comprise a whole brain, a brain region, a neural circuit, or a single neuron. In any of the embodiments herein, the one or more portions of the nervous system from the subject can comprise a dorsolateral prefrontal cortex, frontopolar cortex, anterior cingulate cortex, inferotemporal cortex, visual cortex, auditory cortex, somatosensory cortex, posterior parietal cortex, temporoparietal junction, nucleus accumbens, ventral tegmental area, basal ganglia, orbitofrontal cortex, amygdala, insula, ventromedial prefrontal cortex, hippocampus, entorhinal cortex, parahippcampal gyrus, locus coerulus, reticular formation, ventral striatum, anterior insula, Broca's area, Wernicke's area, medial prefrontal cortex, temporoparietal junction, anterior insula, posterior insula, supplementary motor area, pre-supplementary motor area, or a combination thereof.

In any of the embodiments herein, the one or more neural data structures can correspond to an imaging volume of at most 0.2 cm³. , 0.6 cm³. , 12 cm³. , 700 cm³ or 1000 cm³. . In any of the embodiments herein, the one or more neural data structures can comprise imaging data at an optical resolution.

In any of the embodiments herein, the system can comprise between one and ten ultrasound transducers. In any of the embodiments herein,, the system can further comprise: a remote hub, wherein the remote hub is configured to receive the one or more neural data structures from the controller, and the remote hub is configured to transmit external data to the controller. In any of the embodiments herein, the remote hub can be configured to communicate with a display to provide a user interface for controlling the system. In any of the embodiments herein, the ultrasound transducer and the controller can be configured to communicate wirelessly. In any of the embodiments herein, the ultrasound transducer and the controller can be configured to communicate via Bluetooth, Bluetooth low energy, WiFi, Wi-Fi HaLow, ultra-wideband, impulse-radio ultra-wideband, ultrasonic data transmission, optical data communication, Light Fidelity (li-fi), millimeter wave technologies, wireless chip-to-chip, or a combination thereof.

In any of the embodiments herein, the one or more signals can be configured to coordinate emission of ultrasound waves via the ultrasound transducer and further configured to coordinate receipt of the ultrasound waves. In some embodiments, the controller can comprise a clock, and wherein the one or more signals are sent based on predetermined intervals associated with the clock. In some embodiments, the controller can comprise a central clock, and the one or more ultrasound transducers each comprise a peripheral clock, and wherein the one or more signals correspond to reset signals associated with the central clock. In any of the embodiments herein, the controller can transform the one or more neural data structures. In any of the embodiments herein, the controller can transmit the one or more neural data structures. In any of the embodiments herein, the subject can engage in a clinically relevant behavior while the ultrasound transducer obtains the one or more neural data structures.

In some embodiments, the clinically relevant behavior can comprise activities of daily living, estimates of movement, motion capture, facial expression and response time, self-reported mood, self-reported cognitive state, heart rate, heart rate variability, breathing rate, oxygenation, galvanic skin response, inertial monitoring, or a combination thereof. In any of the embodiments herein, the system can be configured to modify neural activity of the subject based on the one or more neural data structures. In any of the embodiments herein, modifying the neural activity of the subject based on the one or more neural data structures can occur in real-time. In any of the embodiments herein, a real-time modifying of the neural activity of the subject comprises a response time of 5 seconds or less, after receiving the one or more neural data structures. In any of the embodiments herein, the real-time modifying of the neural activity occurs at regular predetermined intervals. In any of the embodiments herein, the one or more neural data structures can comprise imaging data at an optical resolution. In some embodiments, the optical resolution can be at least 20 microns, 25 microns, 50 microns, 100 microns, 150 microns, 200 microns, 250 microns, 300 microns, or 500 microns.

In any of the embodiments herein, the one or more neural data structures can comprise precursor imaging data at a computational superresolution. In any of the embodiments herein, the computational superresolution can be less than the optical resolution. In any of the embodiments herein, the one or more neural data structures can comprise imaging data at an imaging depth. In some embodiments, the imaging depth can be at least 0.04 cm, 0.1 cm, 0.2 cm, 0.5 cm, 2.0 cm, 4.0 cm, 6.0 cm, or 8.0 cm. In any of the embodiments herein, the ultrasound waves can comprise a frequency of at most 100 MHz, 60 MHz, 15 MHz, 7.5 MHz, 5.0 MHz, 2.5 MHz, or 1.0 MHz. In some embodiments, the optical resolution can correspond to approximately the frequency. In any of the embodiments herein, the computational superresolution can correspond to approximately the frequency.

In any of the embodiments herein, the one or more neural data structures can comprise hemodynamic data. In any of the embodiments herein, the one or more neural data structures can comprise neural activity data. In any of the embodiments herein, the one or more neural data structures can comprise neural anatomy data. In any of the embodiments herein, the ultrasound transducer can be an implantable ultrasound transducer. In any of the embodiments herein, the ultrasound transducer can be a non-implantable ultrasound transducer. In some embodiments, the non-implantable ultrasound transducer can comprise a wearable ultrasound transducer.

In any of the embodiments herein, the sono-enhancing contrast agent can comprise a biological sono-enhancing contrast agent, a polymeric sono-enhancing contrast agent, or a pharmaceutical sono-enhancing contrast agent. In some embodiments, the biological sono-enhancing contrast agent can comprise a genetically-encoded sono-enhancing contrast agent. In some embodiments, the genetically-encoded sono-enhancing contrast agent comprises an ultrasonic reporter of calcium, an ultrasonic reporter of voltage, or an ultrasonic reporter of a neurotransmitter, e.g., glutamate, GABA, dopamine, or acetylcholine.

In any of the embodiments herein, the subject can comprise a human subject or a non-human primate subject. In any of the embodiments herein, the subject can have, or can be suspected of having, a neural dysfunction. In some embodiments, the neural dysfunction can comprise a motor dysfunction, a sensory dysfunction, a cognitive dysfunction, a neurological condition, or a neuropsychiatric dysfunction. In some embodiments, the motor dysfunction can comprise a spinal cord injury, amyotrophic lateral sclerosis, locked-in syndrome, stroke, Parkinson's disease, or essential tremor. In any of the embodiments herein, the sensory dysfunction can comprise blindness, deafness, epilepsy, a loss of chemosensation, or a loss of somatosensation. In any of the embodiments herein, the neuropsychiatric dysfunction can comprise clinical depression, schizophrenia, addiction, post-traumatic stress disorder, eating disorder, or sleep disorder.

In any of the embodiments herein, the ultrasound transducer can provide a neural augmenting to the subject. In any of the embodiments herein, the neural augmenting can comprise sensorimotor integrating, sensory augmenting, integrating to a virtual reality, integrating to an augmented reality, modulating a brain state, or integrating to an artificial intelligence.

In some aspects, disclosed herein is an ultrasound transducer configured to emit ultrasound waves, or receive the ultrasound waves, from a whole brain from a subject comprising a sono-enhancing contrast agent, the ultrasound transducer comprising: a sonolucent window; an ultrasonic transducer array, wherein an element in the ultrasonic transducer array can be 100 to 500 microns wide, and the ultrasonic transducer array can comprise at least 100 elements; a control circuit configured to receive or execute instructions for activating a modulating mode or a monitoring mode for the ultrasound transducer; and a processing circuit configured to transform the received ultrasound waves into one or more neural data structures.

In some aspects, disclosed herein is an ultrasound transducer configured to emit ultrasound waves, or receive the ultrasound waves, from a whole brain from a subject comprising a sono-enhancing contrast agent, the ultrasound transducer comprising: a sonolucent window; an ultrasonic transducer array, wherein an element in the ultrasonic transducer array can be 100 to 500 microns wide, the ultrasonic transducer array can comprise at least 100 elements, and the ultrasonic transducer array can be configured to receive or execute instructions for modulating the one or more portions of the nervous system from the subject; and a processing circuit configured to transform the received ultrasound waves into one or more neural data structures.

In some aspects, disclosed herein is an ultrasound transducer configured to emit ultrasound waves, or receive the ultrasound waves, from a whole brain from a subject comprising a sono-enhancing contrast agent, the ultrasound transducer comprising: a sonolucent window; an ultrasonic transducer array, wherein an element in the ultrasonic transducer array can be 100 to 500 microns wide, the ultrasonic transducer array comprises at least 100 elements, and the ultrasonic transducer array can be configured to receive or execute instructions for monitoring the one or more portions of the nervous system from the subject; and a processing circuit configured to transform the received ultrasound waves into one or more neural data structures.

In any of the embodiments herein, the one or more neural data structures can correspond to an imaging volume of at most 100 cm³. , 700 cm³, or 1000 cm³. In any of the embodiments herein, the one or more neural data structures can comprise imaging data at an optical resolution. In some embodiments, the optical resolution can correspond to approximately a width of the element in the ultrasonic transducer array. In some embodiments, the optical resolution can correspond to approximately the width of the element in the ultrasonic transducer array, based on an approximately one-to-one ratio. In any of the embodiments herein, the optical resolution can be at least 100 microns, 150 microns, 250 microns, 300 microns or 500 microns. In any of the embodiments herein, the one or more neural data structures can comprise precursor imaging data at a computational superresolution. In some embodiments, the computational superresolution can correspond to less than the width of the element in the ultrasonic transducer array. In any of the embodiments herein, the computational superresolution can correspond to approximately the width of the element in the ultrasonic transducer array, based on an approximately multi-to-one ratio. In any of the embodiments herein, the one or more neural data structures can comprise imaging data at an imaging depth. In some embodiments, the imaging depth can be at least 4.0 cm, 6.0 cm, or 8.0 cm. In any of the embodiments herein, the ultrasound waves can comprise a frequency of at most 5.0 MHz, 2.5 MHz, or 1.0 MHz. In some embodiments, the optical resolution corresponds to approximately the frequency. In any of the embodiments herein, the computational superresolution can correspond to approximately the frequency.

In some aspects, disclosed herein is an ultrasound transducer configured to emit ultrasound waves, or receive the ultrasound waves, from a brain region or a neural circuit from a subject comprising a sono-enhancing contrast agent, the ultrasound transducer comprising: a sonolucent window; an ultrasonic transducer array, wherein an element in the ultrasonic transducer array can be 50 to 250 microns wide, and the ultrasonic transducer array can comprise at least 100 elements; a control circuit configured to receive or execute instructions for activating a modulating mode or a monitoring mode for the ultrasound transducer; and a processing circuit configured to transform the received ultrasound waves into one or more neural data structures.

In some aspects, disclosed herein is an ultrasound transducer configured to emit ultrasound waves, or receive the ultrasound waves, from a brain region or a neural circuit from a subject comprising a sono-enhancing contrast agent, the ultrasound transducer comprising: a sonolucent window; an ultrasonic transducer array, wherein an element in the ultrasonic transducer array can be 50 to 250 microns wide, the ultrasonic transducer array can comprise at least 100 elements, and the ultrasonic transducer array can be configured to receive or execute instructions for modulating the one or more portions of the nervous system from the subject; and a processing circuit configured to transform the received ultrasound waves into one or more neural data structures.

In some aspects, disclosed herein is an ultrasound transducer configured to emit ultrasound waves, or receive the ultrasound waves, from a brain region or a neural circuit from a subject comprising a sono-enhancing contrast agent, the ultrasound transducer comprising: a sonolucent window; an ultrasonic transducer array, wherein an element in the ultrasonic transducer array can be 50 to 250 microns wide, the ultrasonic transducer array can comprise at least 100 elements, and the ultrasonic transducer array can be configured to receive or execute instructions for monitoring the one or more portions of the nervous system from the subject; and a processing circuit configured to transform the received ultrasound waves into one or more neural data structures.

In any of the embodiments herein, the one or more neural data structures can correspond to an imaging volume of at most 0.6 cm³, 12 cm³, or 100 cm³. In any of the embodiments herein, the one or more neural data structures can comprise imaging data at an optical resolution. In some embodiments, the optical resolution can correspond to approximately a width of the element in the ultrasonic transducer array. In some embodiments, the optical resolution can correspond to approximately the width of the element in the ultrasonic transducer array, based on an approximately one-to-one ratio. In any of the embodiments herein, the optical resolution can be at least 50 microns, 100 microns, 150 microns, 200 microns, or 250 microns. In any of the embodiments herein, the one or more neural data structures can comprise precursor imaging data at a computational superresolution. In some embodiments, the computational superresolution can correspond to less than the width of the element in the ultrasonic transducer array. In any of the embodiments herein, the computational superresolution can correspond to approximately the width of the element in the ultrasonic transducer array, based on an approximately multi-to-one ratio. In any of the embodiments herein, the one or more neural data structures can comprise imaging data at an imaging depth. In some embodiments, the imaging depth can be at least 6.0 cm, or 8.0 cm. In any of the embodiments herein, the ultrasound waves can comprise a frequency of at most 15 MHz, 7.5 MHz, or 5.0 MHz. In some embodiments, the optical resolution can correspond to approximately the frequency. In any of the embodiments herein, the computational superresolution can correspond to approximately the frequency.

In some aspects, disclosed herein is an ultrasound transducer configured to emit ultrasound waves, or receive the ultrasound waves, from a neural circuit or a single neuron from a subject comprising a sono-enhancing contrast agent, the ultrasound transducer comprising: a sonolucent window; an ultrasonic transducer array, wherein an element in the ultrasonic transducer array can be 20 to 50 microns wide, and the ultrasonic transducer array can comprise at least 100 elements; a control circuit configured to receive or execute instructions for activating a modulating mode or a monitoring mode for the ultrasound transducer; and a processing circuit configured to transform the received ultrasound waves into one or more neural data structures.

In some aspects, disclosed herein is an ultrasound transducer configured to emit ultrasound waves, or receive the ultrasound waves, from a neural circuit or a single neuron from a subject comprising a sono-enhancing contrast agent, the ultrasound transducer comprising: a sonolucent window; an ultrasonic transducer array, wherein an element in the ultrasonic transducer array is 20 to 50 microns wide, the ultrasonic transducer array comprises at least 100 elements, and the ultrasonic transducer array is configured to receive or execute instructions for modulating the one or more portions of the nervous system from the subject; and a processing circuit configured to transform the received ultrasound waves into one or more neural data structures.

In some aspects, disclosed herein is an ultrasound transducer configured to emit ultrasound waves, or receive the ultrasound waves, from a neural circuit or a single neuron from a subject comprising a sono-enhancing contrast agent, the ultrasound transducer comprising: a sonolucent window; an ultrasonic transducer array, wherein an element in the transducer array can be 20 to 50 microns wide, the ultrasonic transducer array can comprise at least 100 elements, and the ultrasonic transducer array can be configured to receive or execute instructions for monitoring the one or more portions of the nervous system from the subject; and a processing circuit configured to transform the received ultrasound waves into one or more neural data structures.

In any of the embodiments herein, the one or more neural data structures can correspond to an imaging volume of at most 0.2 cm³ or 0.6 cm³. In any of the embodiments herein, the one or more neural data structures can comprise imaging data at an optical resolution. In some embodiments, the optical resolution can correspond to approximately a width of the element in the ultrasonic transducer array. In some embodiments, the optical resolution can correspond to approximately the width of the element in the ultrasonic transducer array, based on an approximately one-to-one ratio. In any of the embodiments herein, the optical resolution can be at most 50 microns, 25 microns, or 20 microns. In any of the embodiments herein, the one or more neural data structures can comprise precursor imaging data at a computational superresolution. In some embodiments, the computational superresolution can correspond to less than the width of the element in the ultrasonic transducer array. In any of the embodiments herein, the computational superresolution can correspond to approximately the width of the element in the ultrasonic transducer array, based on an approximately multi-to-one ratio. In any of the embodiments herein, the one or more neural data structures can comprise imaging data at an imaging depth. In some embodiments, the imaging depth can be at most 0.2 cm, 0.5 cm, or 2.0 cm. In any of the embodiments herein, the ultrasound waves can comprise a frequency of at least 15 MHz, 60 MHz, or 100 MHz. In some embodiments, the optical resolution can correspond to approximately the frequency. In any of the embodiments herein, the computational superresolution can correspond to approximately the frequency.

In any of the embodiments herein, the one or more neural data structures can comprise hemodynamic data. In any of the embodiments herein, the one or more neural data structures can comprise neural activity data. In any of the embodiments herein, the one or more neural data structures can comprise neural anatomy data.

In any of the embodiments herein, the element in the ultrasonic transducer array can be circular, elliptical, square, rectangular, hexagonal, regular, or irregular, in shape. In any of the embodiments herein, the ultrasonic transducer array can be a capacitive micromachined ultrasonic transducer (CMUT) array or a piezoelectric micromachined ultrasonic transducer (PMUT) array. In some embodiments, the CMUT array can comprise a flat membrane CMUT, a curved membrane CMUT, a dual-electrode CMUT, a collapsed-mode CMUT, a wafer-bonded CMUT, a surface-micromachined CMUT, a CMUT-on-CMOS integrated device, a piston-shaped CMUT, an annular array CMUT, or a flexible CMUT. In some embodiments, the PMUT array can comprise a unimorph PMUT, a bimorph PMUT, a dome-shaped PMUT, a suspended membrane PMUT, an aluminum nitride PMUT, a lead zirconate titanate PMUT, a scandium-doped aluminum nitride PMUT, a lithium niobate PMUT, a polyvinylidene fluoride PMUT, a thin-film PMUT, or a flexible PMUT.

In any of the embodiments herein, the ultrasound transducer can be an implantable ultrasound transducer. In any of the embodiments herein, the ultrasound transducer can be a non-implantable ultrasound transducer. In some embodiments, the non-implantable ultrasound transducer can comprise a wearable ultrasound transducer.

In any of the embodiments herein, the sono-enhancing contrast agent can comprise a biological sono-enhancing contrast agent, a polymeric sono-enhancing contrast agent, or a pharmaceutical sono-enhancing contrast agent. In some embodiments, the biological sono-enhancing contrast agent can comprise a genetically-encoded sono-enhancing contrast agent. In some embodiments, the genetically-encoded sono-enhancing contrast agent can comprise an ultrasonic reporter of calcium, an ultrasonic reporter of voltage, or an ultrasonic reporter of a neurotransmitter, e.g., glutamate, GABA, dopamine, or acetylcholine.

In any of the embodiments herein, the subject can comprise a human subject or a non-human primate subject. In any of the embodiments herein, the subject can have, or can be suspected of having, a neural dysfunction. In some embodiments, the neural dysfunction can comprise a motor dysfunction, a sensory dysfunction, a cognitive dysfunction, a neurological condition, or a neuropsychiatric dysfunction. In some embodiments, the motor dysfunction can comprise a spinal cord injury, amyotrophic lateral sclerosis, locked-in syndrome, stroke, Parkinson's disease, or essential tremor. In any of the embodiments herein, the sensory dysfunction can comprise blindness, deafness, epilepsy, a loss of chemosensation, or a loss of somatosensation. In any of the embodiments herein, the neuropsychiatric dysfunction can comprise clinical depression, schizophrenia, addiction, post-traumatic stress disorder, eating disorder, or sleep disorder.

In any of the embodiments herein, the ultrasound transducer can provide a neural augmenting to the subject. In any of the embodiments herein, the neural augmenting can comprise sensorimotor integrating, sensory augmenting, integrating to a virtual reality, integrating to an augmented reality, modulating a brain state, or integrating to an artificial intelligence.

In some aspects, disclosed herein is a method of modulating or monitoring a whole brain of a subject comprising a sono-enhancing contrast agent, comprising: emitting ultrasound waves to the whole brain, from an ultrasound transducer corresponding to the ultrasound transducer of any of the embodiments herein; sono-enhancing the emitted ultrasound waves, via the sono-enhancing contrast agent in the subject, to form sono-enhanced ultrasound waves; receiving the sono-enhanced ultrasound waves from the subject, by the ultrasound transducer; and determining one or more neural data structures, using one or more processors, based on the received sono-enhanced ultrasound waves.

In some embodiments, the ultrasound transducer can comprise an ultrasonic transducer array, wherein an element in the ultrasonic transducer array can be 100 to 500 microns wide. In any of the embodiments herein, the one or more neural data structures can correspond to an imaging volume of at most 100 cm³, 700 cm³, or 1000 cm³. In any of the embodiments herein, the one or more neural data structures can comprise imaging data at an optical resolution, wherein the optical resolution is at least 100 microns, 150 microns, 250 microns, 300 microns or 500 microns. In any of the embodiments herein, the one or more neural data structures can comprise imaging data at an imaging depth, wherein the imaging depth is at least 4.0 cm, 6.0 cm, or 8.0 cm. In any of the embodiments herein, the ultrasound waves can comprise a frequency of at most 5.0 MHz, 2.5 MHz, or 1.0 MHz.

In some aspects, disclosed herein is a method of modulating or monitoring a brain region or a neural circuit of a subject comprising a sono-enhancing contrast agent, comprising: emitting ultrasound waves to the brain region or the neural circuit, from an ultrasound transducer corresponding to the ultrasound transducer of any of the embodiments herein; sono-enhancing the emitted ultrasound waves, via the sono-enhancing contrast agent in the subject, to form sono-enhanced ultrasound waves; receiving the sono-enhanced ultrasound waves from the subject, by the ultrasound transducer; and determining one or more neural data structures, using one or more processors, based on the received sono-enhanced ultrasound waves.

In some embodiments, the ultrasound transducer can comprise an ultrasonic transducer array, wherein an element in the ultrasonic transducer array is 50 to 250 microns wide. In any of the embodiments herein, the one or more neural data structures can correspond to an imaging volume of at most 0.6 cm³, 12 cm³, or 100 cm³. In any of the embodiments herein, the one or more neural data structures can comprise imaging data at an optical resolution, wherein the optical resolution is at least 50 microns, 100 microns, 150 microns, 200 microns, or 250 microns. In any of the embodiments herein, the one or more neural data structures can comprise imaging data at an imaging depth, wherein the imaging depth is at least 2.0 cm, or 4.0 cm. In any of the embodiments herein, the ultrasound waves can comprise a frequency of at most 15 MHz, 7.5 MHz, or 5.0 MHz.

In some aspects, disclosed herein is a method of modulating or monitoring a neural circuit or a single neuron of a subject comprising a sono-enhancing contrast agent, comprising: emitting ultrasound waves to the neural circuit or the single neuron, from an ultrasound transducer corresponding to the ultrasound transducer of any of the embodiments herein; sono-enhancing the emitted ultrasound waves, via the sono-enhancing contrast agent in the subject, to form sono-enhanced ultrasound waves; receiving the sono-enhanced ultrasound waves from the subject, by the ultrasound transducer; and determining one or more neural data structures, using one or more processors, based on the received sono-enhanced ultrasound waves.

In some embodiments, the ultrasound transducer can comprise an ultrasonic transducer array, wherein an element in the ultrasonic transducer array is 20 to 50 microns wide. In any of the embodiments herein, the one or more neural data structures can correspond to an imaging volume of at most 0.2 cm³, or 0.6 cm³. In any of the embodiments herein, the one or more neural data structures can comprise imaging data at an optical resolution, wherein the optical resolution is at least 20 microns, 25 microns, or 50 microns. In any of the embodiments herein, the one or more neural data structures can comprise imaging data at an imaging depth, wherein the imaging depth is at least 0.04 cm, 0.51 cm, or 0.4 cm. In any of the embodiments herein, the ultrasound waves can comprise a frequency of at most 100 MHz, 60 MHz, or 15 MHz. In any of the embodiments herein, the one or more neural data structures can comprise precursor imaging data at a computational superresolution. In some embodiments, the computational superresolution can correspond to less than a width of the element in the ultrasonic transducer array.

In any of the embodiments herein, the one or more neural data structures can comprise hemodynamic data. In any of the embodiments herein, the one or more neural data structures can comprise neural activity data. In any of the embodiments herein, the one or more neural data structures can comprise neural anatomy data.

In any of the embodiments herein, the element in the ultrasonic transducer array can be circular, elliptical, square, rectangular, hexagonal, regular, or irregular, in shape. In any of the embodiments herein, the ultrasonic transducer array can be a capacitive micromachined ultrasonic transducer (CMUT) array or a piezoelectric micromachined ultrasonic transducer (PMUT) array. In some embodiments, the CMUT array can comprise a flat membrane CMUT, a curved membrane CMUT, a dual-electrode CMUT, a collapsed-mode CMUT, a wafer-bonded CMUT, a surface-micromachined CMUT, a CMUT-on-CMOS integrated device, a piston-shaped CMUT, an annular array CMUT, or a flexible CMUT. In some embodiments, the PMUT array can comprise a unimorph PMUT, a bimorph PMUT, a dome-shaped PMUT, a suspended membrane PMUT, an aluminum nitride PMUT, a lead zirconate titanate PMUT, a scandium-doped aluminum nitride PMUT, a lithium niobate PMUT, a polyvinylidene fluoride PMUT, a thin-film PMUT, or a flexible PMUT.

In any of the embodiments herein, the ultrasound transducer can be an implantable ultrasound transducer. In any of the embodiments herein, the ultrasound transducer can be a non-implantable ultrasound transducer. In some embodiments, the non-implantable ultrasound transducer can comprise a wearable ultrasound transducer.

In any of the embodiments herein, the sono-enhancing contrast agent can comprise a biological sono-enhancing contrast agent, a polymeric sono-enhancing contrast agent, or a pharmaceutical sono-enhancing contrast agent. In any of the embodiments herein, the biological sono-enhancing contrast agent can comprise a genetically-encoded sono-enhancing contrast agent. In any of the embodiments herein, the genetically-encoded sono-enhancing contrast agent can comprise an ultrasonic reporter of calcium, an ultrasonic reporter of voltage, or an ultrasonic reporter of a neurotransmitter, e.g., glutamate, GABA, dopamine, or acetylcholine.

In any of the embodiments herein, the subject can comprise a human subject or a non-human primate subject. In any of the embodiments herein, the subject can have, or can be suspected of having, a neural dysfunction. In some embodiments, the neural dysfunction can comprise a motor dysfunction, a sensory dysfunction, a cognitive dysfunction, a neurological condition, or a neuropsychiatric dysfunction. In some embodiments, the motor dysfunction can comprise a spinal cord injury, amyotrophic lateral sclerosis, locked-in syndrome, stroke, Parkinson's disease, or essential tremor. In any of the embodiments herein, the sensory dysfunction can comprise blindness, deafness, epilepsy, a loss of chemosensation, or a loss of somatosensation. In any of the embodiments herein, the neuropsychiatric dysfunction can comprise clinical depression, schizophrenia, addiction, post-traumatic stress disorder, eating disorder, or sleep disorder.

In any of the embodiments herein, the ultrasound transducer can provide a neural augmenting to the subject. In any of the embodiments herein, the neural augmenting can comprise sensorimotor integrating, sensory augmenting, integrating to a virtual reality, integrating to an augmented reality, modulating a brain state, or integrating to an artificial intelligence.

In some aspects, disclosed herein is a system comprising: one or more processors; and a memory communicatively coupled to the one or more processors and configured to store instructions that, when executed by the one or more processors, can cause the system to: emit ultrasound waves to a whole brain of a subject, from an ultrasound transducer corresponding to the ultrasound transducer of any of the embodiments herein; sono-enhance the emitted ultrasound waves, via the sono-enhancing contrast agent in the subject, to form sono-enhanced ultrasound waves; receive the sono-enhanced ultrasound waves from the subject, by the ultrasound transducer; and determine one or more neural data structures, using one or more processors, based on the received sono-enhanced ultrasound waves. In some embodiments, the ultrasound transducer can comprise an ultrasonic transducer array, wherein an element in the ultrasonic transducer array can be 100 to 500 microns wide. In any of the embodiments herein, the one or more neural data structures can correspond to an imaging volume of at most 100 cm³, 700 cm³, or 1000 cm³. In any of the embodiments herein, the one or more neural data structures can comprise imaging data at an optical resolution, wherein the optical resolution can be at least 100 microns, 150 microns, 250 microns, 300 microns or 500 microns. In any of the embodiments herein, the one or more neural data structures can comprise imaging data at an imaging depth, wherein the imaging depth is at least 4.0 cm, 6.0 cm, or 8.0 cm. In any of the embodiments herein, the ultrasound waves can comprise a frequency of at most 5.0 MHz, 2.5 MHz, or 1.0 MHz.

In some aspects, disclosed herein is a system comprising: one or more processors; and a memory communicatively coupled to the one or more processors and configured to store instructions that, when executed by the one or more processors, can cause the system to: emit ultrasound waves to a brain region or a neural circuit of a subject, from an ultrasound transducer corresponding to the ultrasound transducer of any of the embodiments herein; sono-enhance the emitted ultrasound waves, via the sono-enhancing contrast agent in the subject, to form sono-enhanced ultrasound waves; receive the sono-enhanced ultrasound waves from the subject, by the ultrasound transducer; and determine one or more neural data structures, using one or more processors, based on the received sono-enhanced ultrasound waves. In some embodiments, the ultrasound transducer can comprise an ultrasonic transducer array, wherein an element in the ultrasonic transducer array can be 50 to 250 microns wide. In any of the embodiments herein, the one or more neural data structures can correspond to an imaging volume of at most 0.6 cm³, 12 cm³, or 100 cm³. In any of the embodiments herein, the one or more neural data structures can comprise imaging data at an optical resolution, wherein the optical resolution can be at least 50 microns, 100 microns, 150 microns, 200 microns, or 250 microns. In any of the embodiments herein, the one or more neural data structures can comprise imaging data at an imaging depth, wherein the imaging depth is at least 2.0 cm, or 4.0 cm. In any of the embodiments herein, the ultrasound waves can comprise a frequency of at most 15 MHz, 7.5 MHz, or 5.0 MHz.

In some aspects, disclosed herein is a system comprising: one or more processors; and a memory communicatively coupled to the one or more processors and configured to store instructions that, when executed by the one or more processors, can cause the system to: emit ultrasound waves to a neural circuit or a single neuron of a subject, from an ultrasound transducer corresponding to the ultrasound transducer of any of the embodiments herein; sono-enhance the emitted ultrasound waves, via the sono-enhancing contrast agent in the subject, to form sono-enhanced ultrasound waves; receive the sono-enhanced ultrasound waves from the subject, by the ultrasound transducer; and determine one or more neural data structures, using one or more processors, based on the received sono-enhanced ultrasound waves. In some embodiments, the ultrasound transducer can comprise an ultrasonic transducer array, wherein an element in the ultrasonic transducer array can be 20 to 50 microns wide. In any of the embodiments herein, the one or more neural data structures can correspond to an imaging volume of at most 0.2 cm³, or 0.6 cm³. In any of the embodiments herein, the one or more neural data structures can comprise imaging data at an optical resolution, wherein the optical resolution is at least 20 microns, 25 microns, or 50 microns. In any of the embodiments herein, the one or more neural data structures can comprise imaging data at an imaging depth, wherein the imaging depth is at least 0.04 cm, 0.1 cm, or 0.4 cm. In any of the embodiments herein, the ultrasound waves can comprise a frequency of at most 100 MHz, 60 MHz, or 15 MHz.

In some aspects, disclosed herein is a non-transitory computer-readable storage medium storing one or more programs, the one or more programs comprising instructions, which when executed by one or more processors of a system, can cause the system to: emit ultrasound waves to a whole brain of a subject, from an ultrasound transducer corresponding to the ultrasound transducer of any of the embodiments herein; sono-enhance the emitted ultrasound waves, via the sono-enhancing contrast agent in the subject, to form sono-enhanced ultrasound waves; receive the sono-enhanced ultrasound waves from the subject, by the ultrasound transducer; and determine one or more neural data structures, using one or more processors, based on the received sono-enhanced ultrasound waves. In some embodiments, the ultrasound transducer comprises an ultrasonic transducer array, wherein an element in the ultrasonic transducer array can be 20 to 50 microns wide. In any of the embodiments herein, the one or more neural data structures can correspond to an imaging volume of at most 0.2 cm³, or 0.6 cm³. In any of the embodiments herein, the one or more neural data structures can comprise imaging data at an optical resolution, wherein the optical resolution is at least 20 microns, 25 microns, or 50 microns. In any of the embodiments herein, the one or more neural data structures can comprise imaging data at an imaging depth, wherein the imaging depth is at least 0.04 cm, 0.1 cm, or 0.4 cm. In any of the embodiments herein, the ultrasound waves can comprise a frequency of at most 100 MHz, 60 MHz, or 15 MHz.

In some aspects, disclosed herein is a non-transitory computer-readable storage medium storing one or more programs, the one or more programs comprising instructions, which when executed by one or more processors of a system, can cause the system to: emit ultrasound waves to a brain region or a neural circuit of a subject, from an ultrasound transducer corresponding to the ultrasound transducer of any of the embodiments herein; sono-enhance the emitted ultrasound waves, via the sono-enhancing contrast agent in the subject, to form sono-enhanced ultrasound waves; receive the sono-enhanced ultrasound waves from the subject, by the ultrasound transducer; and determine one or more neural data structures, using one or more processors, based on the received sono-enhanced ultrasound waves. In some embodiments, the ultrasound transducer can comprise an ultrasonic transducer array, wherein an element in the ultrasonic transducer array is 50 to 250 microns wide. In any of the embodiments herein, the one or more neural data structures can correspond to an imaging volume of at most 0.6 cm³, 12 cm³, or 100 cm³. In any of the embodiments herein, the one or more neural data structures can comprise imaging data at an optical resolution, wherein the optical resolution is at least 50 microns, 100 microns, 150 microns, 200 microns, or 250 microns. In any of the embodiments herein, the one or more neural data structures can comprise imaging data at an imaging depth, wherein the imaging depth is at least 2.0 cm, or 4.0 cm. In any of the embodiments herein, the ultrasound waves can comprise a frequency of at most 15 MHz, 7.5 MHz, or 5.0 MHz.

In some aspects, disclosed herein is a non-transitory computer-readable storage medium storing one or more programs, the one or more programs comprising instructions, which when executed by one or more processors of a system, can cause the system to: emit ultrasound waves to a neural circuit or a single neuron of a subject, from an ultrasound transducer corresponding to the ultrasound transducer of any of the embodiments herein; sono-enhance the emitted ultrasound waves, via the sono-enhancing contrast agent in the subject, to form sono-enhanced ultrasound waves; receive the sono-enhanced ultrasound waves from the subject, by the ultrasound transducer; and determine one or more neural data structures, using one or more processors, based on the received sono-enhanced ultrasound waves. In some embodiments, the ultrasound transducer can comprise an ultrasonic transducer array, wherein an element in the ultrasonic transducer array can be 20 to 50 microns wide. In any of the embodiments herein, the one or more neural data structures can correspond to an imaging volume of at most 0.2 cm³, or 0.6 cm³. In any of the embodiments herein, the one or more neural data structures can comprise imaging data at an optical resolution, wherein the optical resolution is at least 20 microns, 25 microns, or 50 microns. In any of the embodiments herein, the one or more neural data structures can comprise imaging data at an imaging depth, wherein the imaging depth is at least 0.04 cm, 0.1 cm, or 0.4 cm. In any of the embodiments herein, the ultrasound waves can comprise a frequency of at most 100 MHz, 60 MHz, or 15 MHz.

In some aspects, disclosed herein is a method for modulating a first ongoing psychophysical state of a subject, based on neural activity from one or more portions of a nervous system of a subject comprising a sono-enhancing contrast agent, comprising: emitting first ultrasound waves to the subject, from an ultrasound transducer corresponding to the ultrasound transducer of any of the embodiments herein; receiving first sono-enhanced ultrasound waves from the subject, wherein the first sono-enhanced ultrasound waves comprise the first emitted ultrasound waves enhanced via the sono-enhancing contrast agent; determining a first plurality of one or more neural data structures, using one or more processors, based on the first received sono-enhanced ultrasound waves; decoding the first plurality into a first high-dimensional goal manifold, via a predictive machine learning algorithm; determining first instructions based on the first high-dimensional goal manifold, via an instruction-determining machine learning algorithm; and modulating the first ongoing psychophysical state of the subject, based on the first instructions.

In some embodiments, the methods described herein can further comprise: emitting second ultrasound waves to the subject, from the ultrasound transducer; receiving second sono-enhanced ultrasound waves from the subject, wherein the second sono-enhanced ultrasound waves comprise the second emitted ultrasound waves enhanced via the sono-enhancing contrast agent; determining a second plurality of one or more neural data structures, using one or more processors, based on the second received sono-enhanced ultrasound waves; decoding the second plurality or the first plurality into a second high-dimensional goal manifold, via a predictive machine learning algorithm; determining second instructions based on the first high-dimensional goal manifold, via an instruction-determining machine learning algorithm; and modulating the second ongoing psychophysical state of the subject, based on the second instructions.

In some aspects, disclosed herein is a method for modulating a first ongoing psychophysical state of a subject, based on neural activity from one or more portions of a nervous system of a subject comprising a sono-enhancing contrast agent, comprising: emitting first ultrasound waves to the subject, from an ultrasound transducer corresponding to the ultrasound transducer of any of the embodiments herein, to the one or more portions of the nervous system; receiving first sono-enhanced ultrasound waves from the subject, wherein the first sono-enhanced ultrasound waves comprise the first emitted ultrasound waves enhanced via the sono-enhancing contrast agent; determining a first plurality of one or more neural data structures, using one or more processors, based on the first received sono-enhanced ultrasound waves; decoding the first plurality, via an instruction-determining machine learning algorithm, into first instructions; and modulating the first ongoing psychophysical state of the subject, based on the first instructions

In some embodiments, the methods disclosed herein can further comprise: emitting second ultrasound waves to the subject, from an ultrasound transducer corresponding to the ultrasound transducer of any of the embodiments herein, to the one or more portions of the nervous system; receiving second sono-enhanced ultrasound waves from the subject, wherein the second sono-enhanced ultrasound waves comprise the second emitted ultrasound waves enhanced via the sono-enhancing contrast agent; determining a second plurality of one or more neural data structures, using one or more processors, based on the second received sono-enhanced ultrasound waves; decoding the second plurality, via an instruction-determining machine learning algorithm, into second instructions; and modulating the second ongoing psychophysical state of the subject, based on the second instructions.

In any of the embodiments herein, the first high-dimensional goal manifold or the second high-dimensional goal manifold comprises dimensions representing, at least in part, predictions across multiple timescales, environmental variables, or personal values of the subject. In some embodiments, the multiple timescales comprise milliseconds-long timescales, seconds-long timescales, minutes-long timescales, hours-long timescales. days-long timescales, weeks-long timescales, months-long timescales, or years-long timescales. The method of any of the embodiments herein, wherein the modulating the first ongoing psychophysical state or the modulating the second ongoing psychophysical state comprises modulating the neural activity of the subject. In some embodiments, the modulating the neural activity of the subject can comprise the emitting of the ultrasound waves to the subject, from the ultrasound transducer. In any of the embodiments herein, the modulating the neural activity of the subject can comprise electro-stimulating the one or more portions of the nervous system of the subject via one or more electrodes. In some embodiments, the electro-stimulating can comprise deep brain stimulation, transcranial magnetic stimulation (TMS), repetitive TMS, vagus nerve stimulation, transcranial direct current stimulation, or electrocorticography.

In any of the embodiments herein, the determining the first plurality can occur before the determining the second plurality. In any of the embodiments herein, the one or more portions of the nervous system from the subject can comprise a whole brain, a brain region, a neural circuit, or a single neuron. In any of the embodiments herein, the one or more portions of the nervous system from the subject can comprise a dorsolateral prefrontal cortex, frontopolar cortex, anterior cingulate cortex, inferotemporal cortex, visual cortex, auditory cortex, somatosensory cortex, posterior parietal cortex, temporoparietal junction, nucleus accumbens, ventral tegmental area, basal ganglia, orbitofrontal cortex, amygdala, insula, ventromedial prefrontal cortex, hippocampus, entorhinal cortex, parahippcampal gyrus, locus coerulus, reticular formation, ventral striatum, anterior insula, Broca's area, Wernicke's area, medial prefrontal cortex, temporoparietal junction, anterior insula, posterior insula, supplementary motor area, pre-supplementary motor area, or a combination thereof.

In any of the embodiments herein, the predictive machine learning algorithm or the instruction-determining machine learning algorithm can be trained on ultrasound imaging data, radiofrequency data, electrophysiology data, functional magnetic imaging (fMRI) data, structural magnetic resonance imaging scan data, diffusion tensor imaging data, computed tomography scan data, or behavioral data. In any of the embodiments herein, the predictive machine learning algorithm or the instruction-determining machine learning algorithm can comprise an artificial neural network, a deep neural network, a convolutional neural network, a generative adversarial network, a deep unfolding network, a tokenizer, a transformer, an encoder, a decoder, an autoencoder, a Markov chain, a hidden Markov model, Bayesian optimization, a dimensionality reduction technique, a generalized linear model, a multi-layer perceptron, or a support vector machine. In any of the embodiments herein, the one or more neural data structures can comprise precursor imaging data at a computational superresolution. In some embodiments, the computational superresolution can correspond to less than a width of the element in the ultrasonic transducer array.

In any of the embodiments herein, the one or more neural data structures can comprise hemodynamic data. In any of the embodiments herein, the one or more neural data structures can comprise neural activity data. In any of the embodiments herein, the one or more neural data structures can comprise neural anatomy data.

In any of the embodiments herein, the element in the ultrasonic transducer array can be circular, elliptical, square, rectangular, hexagonal, regular, or irregular, in shape. In any of the embodiments herein, the ultrasonic transducer array can be a capacitive micromachined ultrasonic transducer (CMUT) array or a piezoelectric micromachined ultrasonic transducer (PMUT) array. In some embodiments, the CMUT array can comprise a flat membrane CMUT, a curved membrane CMUT, a dual-electrode CMUT, a collapsed-mode CMUT, a wafer-bonded CMUT, a surface-micromachined CMUT, a CMUT-on-CMOS integrated device, a piston-shaped CMUT, an annular array CMUT, or a flexible CMUT. In some embodiments, the PMUT array can comprise a unimorph PMUT, a bimorph PMUT, a dome-shaped PMUT, a suspended membrane PMUT, an aluminum nitride PMUT, a lead zirconate titanate PMUT, a scandium-doped aluminum nitride PMUT, a lithium niobate PMUT, a polyvinylidene fluoride PMUT, a thin-film PMUT, or a flexible PMUT.

In any of the embodiments herein, the ultrasound transducer can be an implantable ultrasound transducer. In any of the embodiments herein, the ultrasound transducer can be a non-implantable ultrasound transducer. In some embodiments, the non-implantable ultrasound transducer can comprise a wearable ultrasound transducer.

In any of the embodiments herein, the sono-enhancing contrast agent can comprise a biological sono-enhancing contrast agent, a polymeric sono-enhancing contrast agent, or a pharmaceutical sono-enhancing contrast agent. In some embodiments, the biological sono-enhancing contrast agent can comprise a genetically-encoded sono-enhancing contrast agent. In some embodiments, the genetically-encoded sono-enhancing contrast agent can comprise an ultrasonic reporter of calcium, an ultrasonic reporter of voltage, or an ultrasonic reporter of a neurotransmitter, e.g., glutamate, GABA, dopamine, or acetylcholine.

In any of the embodiments herein, the subject can comprise a human subject or a non-human primate subject. In any of the embodiments herein, the subject can have, or can be suspected of having, a neural dysfunction. In some embodiments, the neural dysfunction can comprise a motor dysfunction, a sensory dysfunction, a cognitive dysfunction, a neurological condition, or a neuropsychiatric dysfunction. In some embodiments, the motor dysfunction can comprise a spinal cord injury, amyotrophic lateral sclerosis, locked-in syndrome, stroke, Parkinson's disease, or essential tremor. In any of the embodiments herein, the sensory dysfunction can comprise blindness, deafness, epilepsy, a loss of chemosensation, or a loss of somatosensation. In any of the embodiments herein, the neuropsychiatric dysfunction can comprise clinical depression, schizophrenia, addiction, post-traumatic stress disorder, eating disorder, or sleep disorder.

In any of the embodiments herein, the ultrasound transducer can provide a neural augmenting to the subject. In any of the embodiments herein, the neural augmenting can comprise sensorimotor integrating, sensory augmenting, integrating to a virtual reality, integrating to an augmented reality, modulating a brain state, or integrating to an artificial intelligence.

In some aspects, disclosed herein is a system comprising: one or more processors; and a memory communicatively coupled to the one or more processors and configured to store instructions that, when executed by the one or more processors, can cause the system to: emit first ultrasound waves to the subject, from an ultrasound transducer corresponding to the ultrasound transducer of any of the embodiments herein; receive first sono-enhanced ultrasound waves from the subject, wherein the first sono-enhanced ultrasound waves comprise the first emitted ultrasound waves enhanced via the sono-enhancing contrast agent; determine a first plurality of one or more neural data structures, using one or more processors, based on the first received sono-enhanced ultrasound waves; decode the first plurality into a first high-dimensional goal manifold, via a predictive machine learning algorithm; determine first instructions based on the first high-dimensional goal manifold, via an instruction-determining machine learning algorithm; and modulate the first ongoing psychophysical state of the subject, based on the first instructions.

In some embodiments, the system can comprise further instructions, that when executed by the one or more processors, can cause the system to: emit second ultrasound waves to the subject, from the ultrasound transducer; receive second sono-enhanced ultrasound waves from the subject, wherein the second sono-enhanced ultrasound waves comprise the second emitted ultrasound waves enhanced via the sono-enhancing contrast agent; determine a second plurality of one or more neural data structures, using one or more processors, based on the second received sono-enhanced ultrasound waves; decode the second plurality or the first plurality into a second high-dimensional goal manifold, via a predictive machine learning algorithm; determine second instructions based on the first high-dimensional goal manifold, via an instruction-determining machine learning algorithm; and modulate the second ongoing psychophysical state of the subject, based on the second instructions.

In some aspects, disclosed herein is a non-transitory computer-readable storage medium storing one or more programs, the one or more programs comprising instructions, which when executed by one or more processors of a system, can cause the system to: emit first ultrasound waves to the subject, from an ultrasound transducer corresponding to the ultrasound transducer of any of the embodiments herein; receive first sono-enhanced ultrasound waves from the subject, wherein the first sono-enhanced ultrasound waves comprise the first emitted ultrasound waves enhanced via the sono-enhancing contrast agent; determine a first plurality of one or more neural data structures, using one or more processors, based on the first received sono-enhanced ultrasound waves; decode the first plurality into a first high-dimensional goal manifold, via a predictive machine learning algorithm; determine first instructions based on the first high-dimensional goal manifold, via an instruction-determining machine learning algorithm; and modulate the first ongoing psychophysical state of the subject, based on the first instructions.

In some embodiments, the system can comprise further instructions,, that when executed by the one or more processors, can cause the system to: emit second ultrasound waves to the subject, from the ultrasound transducer; receive second sono-enhanced ultrasound waves from the subject, wherein the second sono-enhanced ultrasound waves comprise the second emitted ultrasound waves enhanced via the sono-enhancing contrast agent; determine a second plurality of one or more neural data structures, using one or more processors, based on the second received sono-enhanced ultrasound waves; decode the second plurality or the first plurality into a second high-dimensional goal manifold, via a predictive machine learning algorithm; determine second instructions based on the first high-dimensional goal manifold, via an instruction-determining machine learning algorithm; and modulate the second ongoing psychophysical state of the subject, based on the second instructions.

In some aspects, disclosed herein is a system comprising: one or more processors; and a memory communicatively coupled to the one or more processors and configured to store instructions that, when executed by the one or more processors, can cause the system to: emit first ultrasound waves to the subject, from an ultrasound transducer corresponding to the ultrasound transducer of any of the embodiments herein, to the one or more portions of the nervous system; receive first sono-enhanced ultrasound waves from the subject, wherein the first sono-enhanced ultrasound waves comprise the first emitted ultrasound waves enhanced via the sono-enhancing contrast agent; determine a first plurality of one or more neural data structures, using one or more processors, based on the first received sono-enhanced ultrasound waves; decode the first plurality, via an instruction-determining machine learning algorithm, into first instructions; and modulate the first ongoing psychophysical state of the subject, based on the first instructions.

In some embodiments, the system can comprise further instructions, that when executed by the one or more processors, can cause the system to: emit second ultrasound waves to the subject, from an ultrasound transducer corresponding to the ultrasound transducer of any of the embodiments herein, to the one or more portions of the nervous system; receive second sono-enhanced ultrasound waves from the subject, wherein the second sono-enhanced ultrasound waves comprise the second emitted ultrasound waves enhanced via the sono-enhancing contrast agent; determine a second plurality of one or more neural data structures, using one or more processors, based on the second received sono-enhanced ultrasound waves; decode the second plurality, via an instruction-determining machine learning algorithm, into second instructions; and modulate the second ongoing psychophysical state of the subject, based on the second instructions.

In some aspects, disclosed herein is a non-transitory computer-readable storage medium storing one or more programs, the one or more programs comprising instructions, which when executed by one or more processors of a system, can cause the system to: emit first ultrasound waves to the subject, from an ultrasound transducer corresponding to the ultrasound transducer of any of the embodiments herein, to the one or more portions of the nervous system; receive first sono-enhanced ultrasound waves from the subject, wherein the first sono-enhanced ultrasound waves comprise the first emitted ultrasound waves enhanced via the sono-enhancing contrast agent; determine a first plurality of one or more neural data structures, using one or more processors, based on the first received sono-enhanced ultrasound waves; decode the first plurality, via an instruction-determining machine learning algorithm, into first instructions; and modulate the first ongoing psychophysical state of the subject, based on the first instructions.

In some embodiments, the system can comprise further instructions, that when executed by the one or more processors, can cause the system to: emit second ultrasound waves to the subject, from an ultrasound transducer corresponding to the ultrasound transducer of any of the embodiments herein, to the one or more portions of the nervous system; receive second sono-enhanced ultrasound waves from the subject, wherein the second sono-enhanced ultrasound waves comprise the second emitted ultrasound waves enhanced via the sono-enhancing contrast agent; determine a second plurality of one or more neural data structures, using one or more processors, based on the second received sono-enhanced ultrasound waves; decode the second plurality, via an instruction-determining machine learning algorithm, into second instructions; and modulate the second ongoing psychophysical state of the subject, based on the second instructions.

In some aspects, disclosed herein is a method of decoding latent neural states directly from one or more neural data structures, comprising: determining, using one or more processors, the one or more target latent neural states; receiving first ultrasound waves reflected from one or more portions of a nervous system from a subject, at the ultrasound transducer corresponding to any of the embodiments herein; determining one or more first neural data structures from the ultrasound transducer; and decoding the one or more first neural data structures, via a regressor, using the one or more processors, into the one or more target latent neural states, thereby bypassing a beamforming transformation of the one or more neural data structures. In some embodiments, the method can further comprise determining first instructions, using one or more processors, based on the one or more target latent neural states; modulating the one or more portions of the nervous system of the subject, based on the first instructions; receiving second ultrasound waves reflected from the one or more portions of the nervous system from the subject, at the ultrasound transducer; determining one or more second neural data structures from the ultrasound transducer; and decoding the one or more second neural data structures, via the regressor, using the one or more processors, into the one or more target latent neural states, thereby bypassing the beamforming transformation of the one or more neural data structures.

In some aspects, disclosed herein is a method of decoding latent neural states directly from one or more neural data structures, comprising: determining, using one or more processors, the one or more target latent neural states; receiving first ultrasound waves reflected from one or more portions of a nervous system from a subject, at the ultrasound transducer corresponding to any of the embodiments herein; determining one or more first neural data structures from the ultrasound transducer; transforming the one or more first neural data structures into a first non-beamformed intermediate representation; and decoding the first non-beamformed intermediate representation, via a regressor, using the one or more processors, into the one or more target latent neural states, thereby bypassing a beamforming transformation of the first non-beamformed intermediate representation. In some embodiments, the method can further comprise determining first instructions, using one or more processors, based on the one or more target latent neural states; modulating the one or more portions of the nervous system of the subject, based on the first instructions; receiving second ultrasound waves reflected from the one or more portions of the nervous system from the subject, at the ultrasound transducer; determining one or more second neural data structures from the ultrasound transducer; transforming the one or more second neural data structures into a second non-beamformed intermediate representation; and decoding the second non-beamformed intermediate representation, via a regressor, using the one or more processors, into the one or more target latent neural states, thereby bypassing a beamforming transformation of the second non-beamformed intermediate representation.

In any of the embodiments herein, the first non-beamformed intermediate representation or the second non-beamformed intermediate representation can comprise a wavelet decomposed representation, a time-frequency representation, or a dimensionality reduced representation. In some embodiments, the time-frequency representation comprises a short-time Fourier transform. In any of the embodiments herein, the first non-beamformed intermediate representation or the second non-beamformed intermediate representation comprises a registered representation. In some embodiments, the registered representation can comprise a motion compensated representation, a spatiotemporal registered representation, or an adaptively sampled representation. In any of the embodiments herein, the modulating the one or more portions of the nervous system comprises emitting additional ultrasound waves to the subject, from the ultrasound transducer. In any of the embodiments herein, the modulating the neural activity of the subject can comprise electro-stimulating the one or more portions of the nervous system of the subject via one or more electrodes. In any of the embodiments herein, the electro-stimulating can comprise deep brain stimulation, transcranial magnetic stimulation (TMS), repetitive TMS, vagus nerve stimulation, transcranial direct current stimulation, or electrocorticography.

In any of the embodiments herein, the regressor can comprise a linear regressor. In some embodiments, the linear regressor can comprise an ordinary least squares regressor, a ridge regressor, a lasso regressor, an elastic net regressor, a partial least squares regressor, a principal component regressor, a weighted least squares regressor, or a least angle regressor. In any of the embodiments herein, the regressor can comprise a non-linear regressor. In some embodiments, the non-linear regressor can comprise a polynomial regressor, a decision tree regressor, a random forest regressor, a logistic regressor, a support vector regressor with non-linear kernels, a neural network, a Gaussian process, a k-nearest neighbor regressor, a spline regressor, kernel ridge regressor, a generalized additive model, a multivariate adaptive regression spline regressor, a kernel regressor, a local regressor, a radial basis function network, an isotonic regressor, a projection pursuit regressor, a quantile regression forest, a sequential state estimator, a Kalman filter, or a multi-task learning regressor. In any of the embodiments herein, the one or more neural data structures can comprise radiofrequency data, in-phase or quadrature (IQ) data, or an envelope of the radiofrequency data.

In any of the embodiments herein, the one or more portions of the nervous system from the subject comprises a whole brain, a brain region, a neural circuit, or a single neuron. In any of the embodiments herein, the one or more portions of the nervous system from the subject can comprise a dorsolateral prefrontal cortex, frontopolar cortex, anterior cingulate cortex, inferotemporal cortex, visual cortex, auditory cortex, somatosensory cortex, posterior parietal cortex, temporoparietal junction, nucleus accumbens, ventral tegmental area, basal ganglia, orbitofrontal cortex, amygdala, insula, ventromedial prefrontal cortex, hippocampus, entorhinal cortex, parahippcampal gyrus, locus coerulus, reticular formation, ventral striatum, anterior insula, Broca's area, Wernicke's area, medial prefrontal cortex, temporoparietal junction, anterior insula, posterior insula, supplementary motor area, pre-supplementary motor area, or a combination thereof.

In any of the embodiments herein, the ultrasound transducer can comprise an ultrasonic transducer array. In some embodiments, the ultrasonic transducer array can be a capacitive micromachined ultrasonic transducer (CMUT) array or a piezoelectric micromachined ultrasonic transducer (PMUT) array. In some embodiments, the CMUT array can comprise a flat membrane CMUT, a curved membrane CMUT, a dual-electrode CMUT, a collapsed-mode CMUT, a wafer-bonded CMUT, a surface-micromachined CMUT, a CMUT-on-CMOS integrated device, a piston-shaped CMUT, an annular array CMUT, or a flexible CMUT. In some embodiments, the PMUT array can comprise a unimorph PMUT, a bimorph PMUT, a dome-shaped PMUT, a suspended membrane PMUT, an aluminum nitride PMUT, a lead zirconate titanate PMUT, a scandium-doped aluminum nitride PMUT, a lithium niobate PMUT, a polyvinylidene fluoride PMUT, a thin-film PMUT, or a flexible PMUT. In any of the embodiments herein, the ultrasound transducer can be an implantable ultrasound transducer. In any of the embodiments herein, the ultrasound transducer can be a non-implantable ultrasound transducer. In some embodiments, the non-implantable ultrasound transducer can comprise a wearable ultrasound transducer.

In any of the embodiments herein, the subject can comprise a sono-enhancing contrast agent. In some embodiments, the sono-enhancing contrast agent can comprise a biological sono-enhancing contrast agent, a polymeric sono-enhancing contrast agent, or a pharmaceutical sono-enhancing contrast agent. In some embodiments, the biological sono-enhancing contrast agent can comprise a genetically-encoded sono-enhancing contrast agent. In some embodiments, the genetically-encoded sono-enhancing contrast agent can comprise an ultrasonic reporter of calcium, an ultrasonic reporter of voltage, or an ultrasonic reporter of a neurotransmitter. In any of the embodiments herein, the subject can comprise a human subject or a non-human primate subject. In any of the embodiments herein, the subject can have, or can be suspected of having a neural dysfunction. In some embodiments, the neural dysfunction can comprise a motor dysfunction, a sensory dysfunction, a cognitive dysfunction, a neurological condition, or a neuropsychiatric dysfunction. In some embodiments, the motor dysfunction can comprise a spinal cord injury, amyotrophic lateral sclerosis, locked-in syndrome, stroke, Parkinson's disease, or essential tremor. In any of the embodiments herein, the sensory dysfunction can comprise blindness, deafness, epilepsy, a loss of chemosensation, or a loss of somatosensation. In any of the embodiments herein, the neuropsychiatric dysfunction can comprise clinical depression, schizophrenia, addiction, post-traumatic stress disorder, eating disorder, or sleep disorder.

In some aspects, disclosed herein is a system comprising: one or more processors; and a memory communicatively coupled to the one or more processors and configured to store instructions that, when executed by the one or more processors, can cause the system to: determine, using one or more processors, the one or more target latent neural states; receive first ultrasound waves reflected from one or more portions of a nervous system from a subject, at the ultrasound transducer corresponding to any of the embodiments herein; determine one or more first neural data structures from the ultrasound transducer; and decode the one or more first neural data structures, via a regressor, using the one or more processors, into the one or more target latent neural states, thereby bypassing a beamforming transformation of the one or more neural data structures. In some embodiments, the system can comprise further instructions, that when executed by the one or more processors, can cause the system to: determine first instructions, using one or more processors, based on the one or more target latent neural states; modulate the one or more portions of the nervous system of the subject, based on the first instructions; receive second ultrasound waves reflected from the one or more portions of the nervous system from the subject, at the ultrasound transducer; determine one or more second neural data structures from the ultrasound transducer; and decode the one or more second neural data structures, via the regressor, using the one or more processors, into the one or more target latent neural states, thereby bypassing the beamforming transformation of the one or more neural data structures.

In some aspects, disclosed herein is a system comprising: one or more processors; and a memory communicatively coupled to the one or more processors and configured to store instructions that, when executed by the one or more processors, can cause the system to: determine, using one or more processors, the one or more target latent neural states; receive first ultrasound waves reflected from one or more portions of a nervous system from a subject, at the ultrasound transducer corresponding to any of the embodiments herein; determine one or more first neural data structures from the ultrasound transducer; transform the one or more first neural data structures into a first non-beamformed intermediate representation; and decode the first non-beamformed intermediate representation, via a regressor, using the one or more processors, into the one or more target latent neural states, thereby bypassing a beamforming transformation of the first non-beamformed intermediate representation. In some embodiments, the system can comprise further instructions, that when executed by the one or more processors, can cause the system to: determine first instructions, using one or more processors, based on the one or more target latent neural states; modulate the one or more portions of the nervous system of the subject, based on the first instructions; receive second ultrasound waves reflected from the one or more portions of the nervous system from the subject, at the ultrasound transducer; determine one or more second neural data structures from the ultrasound transducer; transform the one or more second neural data structures into a second non-beamformed intermediate representation; and decode the second non-beamformed intermediate representation, via a regressor, using the one or more processors, into the one or more target latent neural states, thereby bypassing a beamforming transformation of the second non-beamformed intermediate representation.

In some aspects, disclosed herein is a non-transitory computer-readable storage medium storing one or more programs, the one or more programs comprising instructions, which when executed by one or more processors of a system, cause the system to: determine, using one or more processors, the one or more target latent neural states; receive first ultrasound waves reflected from one or more portions of a nervous system from a subject, at the ultrasound transducer corresponding to any of the embodiments herein; determine one or more first neural data structures from the ultrasound transducer; and decode the one or more first neural data structures, via a regressor, using the one or more processors, into the one or more target latent neural states, thereby bypassing a beamforming transformation of the one or more neural data structures.

In some embodiments, the non-transitory computer-readable storage medium can comprise further instructions, that when executed by the one or more processors, can cause the system to: determine first instructions, using one or more processors, based on the one or more target latent neural states; modulate the one or more portions of the nervous system of the subject, based on the first instructions; receive second ultrasound waves reflected from the one or more portions of the nervous system from the subject, at the ultrasound transducer; determine one or more second neural data structures from the ultrasound transducer; and decode the one or more second neural data structures, via the regressor, using the one or more processors, into the one or more target latent neural states, thereby bypassing the beamforming transformation of the one or more neural data structures.

In some aspects, a non-transitory computer-readable storage medium storing one or more programs, the one or more programs comprising instructions, which when executed by one or more processors of a system, can cause the system to: determine, using one or more processors, the one or more target latent neural states; receive first ultrasound waves reflected from one or more portions of a nervous system from a subject, at the ultrasound transducer corresponding to any of the embodiments herein; determine one or more first neural data structures from the ultrasound transducer; transform the one or more first neural data structures into a first non-beamformed intermediate representation; and decode the first non-beamformed intermediate representation, via a regressor, using the one or more processors, into the one or more target latent neural states, thereby bypassing a beamforming transformation of the first non-beamformed intermediate representation. In some embodiments, the non-transitory computer-readable storage medium can comprise further instructions, that when executed by the one or more processors, can cause the system to: determine first instructions, using one or more processors, based on the one or more target latent neural states; modulate the one or more portions of the nervous system of the subject, based on the first instructions; receive second ultrasound waves reflected from the one or more portions of the nervous system from the subject, at the ultrasound transducer; determine one or more second neural data structures from the ultrasound transducer; transform the one or more second neural data structures into a second non-beamformed intermediate representation; and decode the second non-beamformed intermediate representation, via a regressor, using the one or more processors, into the one or more target latent neural states, thereby bypassing a beamforming transformation of the second non-beamformed intermediate representation.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1A and FIG. 1B provide exploded view renderings of an example ultrasound transducer device, according to some embodiments.

FIG. 2A provides a diagram illustrating the use of implantable ultrasound transducers in a subject.

FIG. 2B provides a diagram illustrating the positioning of an implantable ultrasound transducer, relative to a subject.

FIG. 3 provides an exemplary workflow for ultrasound-based monitoring or modulating of a whole brain for a subject comprising a sono-enhancing contrast agent, according to some embodiments.

FIG. 4 provides an exemplary workflow for ultrasound-based monitoring or modulating of one or more brain regions or neural circuits for a subject comprising a sono-enhancing contrast agent, according to some embodiments.

FIG. 5 provides an exemplary workflow for ultrasound-based monitoring or modulating of one or more neural circuits or single neurons for a subject comprising a sono-enhancing contrast agent, according to some embodiments.

FIG. 6 provides an exemplary workflow for modulating an ongoing physiological state of a subject comprising a sono-enhancing contrast agent. The modulating can be based on predicting an expected physiological state of the subject, and neural activity of the subject, wherein the neural activity is based on received ultrasound waves.

FIG. 7 provides an additional exemplary workflow for modulating an ongoing physiological state of a subject comprising a sono-enhancing contrast agent. The modulating can be based on predicting an expected physiological state of the subject, and neural activity of the subject, wherein the neural activity is based on received ultrasound waves.

FIG. 8 provides an exemplary workflow for modulating an ongoing physiological state of a subject comprising a sono-enhancing contrast agent. The modulating can be based on neural activity of the subject, wherein the neural activity is based on received ultrasound waves.

FIG. 9 provides an exemplary workflow for modulating an ongoing physiological state of a subject comprising a sono-enhancing contrast agent. The modulating can be based on neural activity of the subject, wherein the neural activity is based on received ultrasound waves.

FIG. 10 provides an exemplary workflow for decoding latent neural states directly from one or more neural data structures.

FIG. 11 provides an additional exemplary workflow for decoding latent neural states directly from one or more neural data structures.

FIG. 12 provides an exemplary workflow for decoding latent neural states from one or more neural data structures, by transforming the one or more neural data structures into a non-beamformed intermediate representation.

FIG. 13 provides an additional exemplary workflow for decoding latent neural states from one or more neural data structures, by transforming the one or more neural data structures into a non-beamformed intermediate representation.

FIG. 14 depicts an example computing device or system, according to some embodiments.

FIG. 15 depicts an example computer system or computer network, according to some embodiments.

FIG. 16 provides a diagram illustrating scaling between monitoring or modulating penetration depth, imaging, or modulating resolution, and ultrasound frequency.

FIG. 17A provides a diagram illustrating the imaging or modulating parameters for an ultrasound transducer optimized for operating at a single-neuron scale.

FIG. 17B provides a diagram illustrating the imaging or modulating parameters for an ultrasound transducer optimized for operating at a brain-wide scale.

FIG. 18 provides a diagram illustrating the use of the ultrasound transducers optimized for brain-wide monitoring or modulating of a nervous system comprising sono-enhancing contrast agents.

FIG. 19 provides a diagram illustrating the use of the ultrasound transducers optimized for monitoring or modulating brain regions or neural circuits, for a nervous system comprising sono-enhancing contrast agents.

FIG. 20 provides a diagram illustrating the use of the ultrasound transducers optimized for monitoring or modulating neural circuits or single neurons for a nervous system comprising sono-enhancing contrast agents.

FIG. 21 provides point scatter simulation data for validating the decoding of target latent neural states.

### DETAILED DESCRIPTION

Disclosed herein are methods and systems for monitoring or modulating one or more portions of the nervous system of a subject, using ultrasound. The subject can comprise a sono-enhancing contrast agent, which can improve the signal-to-noise ratio of ultrasound waves detected by an ultrasound transducer.

Neural disorders manifest from dysfunctions occurring across multiple scales of the nervous system. Current neurotechnology lacks a unified and scalable platform for neural monitoring or modulation. Clinicians and researchers must resort to completely different technological modalities when studying or treating the nervous system at varying scales. The technologies required for investigating the whole brain typically differ from those employed for examining specific brain regions, neural circuits, or single neurons. This technological fragmentation increases procedural complexities, cost burdens, and often requires subjects to undergo multiple invasive procedures. Furthermore, the absence of a unified approach creates significant challenges in integrating data across these disparate technological platforms.

This disclosure provides a solution through a unified technological framework that functions effectively across the full spectrum of neural scales, from entire brain monitoring to single-neuron interaction. The disclosed devices, systems, and methods utilize specialized ultrasound transducers that operate on shared principles and compatible data processing architectures. Each transducer is specifically engineered for a particular neural scale: ultrasonic transducer arrays with larger elements (100-500 microns) for whole-brain applications, arrays with medium-sized elements (50-250 microns) for brain regions and neural circuits, and arrays with small elements (20-50 microns) for neural circuits and individual neurons.

Beyond element size optimization, these ultrasound transducers are further tailored to their target neural scales through the emission of scale-appropriate ultrasound frequencies. Lower frequencies with greater penetration capability (e.g., 2.5-5.0 MHz) enable whole-brain monitoring or modulation at broader resolutions. Intermediate frequencies (e.g., 5.0-15 MHz) facilitate regional monitoring or modulation. Higher frequencies with reduced penetration (e.g., 15-100 MHz) allow for high-resolution circuit and cellular monitoring. For each scale-optimized ultrasound transducer, the combination of the specialized frequency range and the specialized range of micron widths contribute to the scale optimization of the scale-optimized ultrasound transducer. Despite these scale-specific optimizations, all transducers incorporate a common technological architecture, interface with identical control systems, and produce neural data structures in compatible formats.

A significant challenge for ultrasound and similar volumetric imaging techniques is the supralinear decrease in signal-to-noise ratio that occurs at smaller imaging volumes and higher resolutions. This decrease can render imaging signals unrecoverable or neuromodulation ineffective when using transducers optimized for intermediate or high frequencies. The present disclosure addresses this challenge through sono-enhancing contrast agents. These agents, when activated by emitted ultrasound waves, produce strongly scattered ultrasound waves that can be readily detected and processed by the transducer. This approach maintains high-quality neural monitoring or modulation even at the finest spatial resolutions or deepest tissue penetrations. The sono-enhancing contrast agents may be biological in nature, including genetically encoded acoustic proteins such as ultrasonic reporters of calcium, ultrasonic reporters of voltage, or ultrasonic reporters of a neurotransmitter, e.g., glutamate, GABA, dopamine, or acetylcholine.

A scalable platform for ultrasound transducers that are optimized to specific neural scales, and yet maintain integration to a common technological ecosystem, represents a significant improvement over existing technologies. For example, clinicians that study subjects at a brain-wide scale commonly employ functional magnetic resonance imaging (fMRI), but employ a completely different technological modality, such as electrophysiology, when studying subjects at a neural circuit-level or single neuron-level scale. The use of multiple and entirely distinct instrumentations result in increased invasiveness to the subject, and increased cost to the subject and clinician. For example, an fMRI machine and an electrophysiology set-up require two completely distinct technical skillsets to operate, and two completely distinct technical skillsets to maintain and repair. The sum of these operation and maintenance costs across technological modalities contribute to immense costs to both the subject and the clinician. In contrast, the scale-optimized ultrasound transducers allow for a singular technological modality that simultaneously supports the monitoring and modulating of nervous systems across disparate scales. As a result, a minimal technical skillset can be used for operating, maintaining, and repairing the ultrasound transducer technology.

Furthermore, the use of different technological modalities result in disparate data structures that require intensive time and money to process cohesively. For instance, the task of merely aligning fMRI data to electrophysiology data across both time and space constitute a highly challenging technical problem—at the very least, the sampling methods between the modalities are completely different, and the spatial resolutions of fMRI and electrophysiology are typically incommensurable. In contrast, the embodiments of the present disclosure describe an elegant and scalable solution for studying nervous systems across a broad range of scales, by using scale-optimized ultrasound transducers. The consistent use of ultrasound transducers results in a common technological modality that can output compatible data structures, and comparable experimental parameters, even if data is acquired across disparate neural scales that range from the brain-wide scale to the single-neuron scale.

The ultrasound transducers described in the present disclosure does not sacrifice imaging or modulating resolution, despite the transducers' use across varying neural scales. The maintaining of an optimum resolution at each of the varying neural scales is accomplished, in part, by the use of a sono-enhancing contrast agent. The sono-enhancing contrast agent can include genetically-encoded contrast agents that can be delivered to the subject, e.g., via intravenous application. The sono-enhancing contrast agents can, upon receiving ultrasound waves at a first desired frequency range, emit readily detectable ultrasound waves via a second desired frequency range (which can, at least, in part, overlap with the first desired frequency range). The sono-enhancing contrast agents can, in some embodiments, operate based on the buckling properties of gas vesicles. By using sono-enhancing contrast agents, clinicians and scientists can increase the signal-to-noise ratio to result in usable data, regardless of the scale of the ultrasound-based imaging or neuromodulating. The leveraging of sono-enhancing contrast agents, including those from the fields of molecular biology and molecular engineering, in combination with developments from a completely distinct field—namely, from electrical engineering, via e.g., the altering of element widths in ultrasonic transducer arrays—to achieve a highly scalable platform for monitoring or modulating a nervous system, constitutes a significant improvement over existing technologies, which are often limited to improvements in a single field.

The scalability alone of the ultrasound transducers and their associated methods and systems represents a considerable improvement over existing technologies. The present disclosure, however, provides further advantages over existing technologies in that the same ultrasound transducers can be used for both monitoring and modulating the nervous system of the subject. For example, the circuitry of a single ultrasound device can be configured to either emit and/or receive ultrasound waves corresponding to a modulating mode, or can be configured to either emit and/or receive ultrasound waves corresponding to a monitoring mode. Alternatively, a more specialized ultrasound device can be used, wherein the ultrasound device is limited to emitting and/or receiving ultrasound waves corresponding to only the monitoring of a nervous system, or corresponding to only the modulating of the nervous system. Multiple of these ultrasound transducers—regardless of whether a given ultrasound transducer can only monitor or modulate the nervous system, or are configured to either monitor or modulate the nervous system—can be used together, to efficiently monitor and/or modulate one or more portions of a nervous system of a subject. The flexibility of such a system comprising multiple of these ultrasound transducers marks a considerable improvement over other technological modalities, in that even a single ultrasound transducer—let alone, multiple—can be used for monitoring or modulating a nervous system. The devices, methods, and systems described herein allow for a streamlined and efficient operation, in that a singular technological platform can both monitor or modulate the nervous system, in addition to being able to modulate or monitor the nervous system across disparate neural scales. Existing methodologies fail to offer such a streamlined, yet encompassing solution, for studying the nervous system.

The integration of scale-optimized ultrasound transducers with sono-enhancing contrast agents establishes a unified and reliable platform for nervous system monitoring or modulation. The disclosed devices, systems, and methods provide a scalable and dependable ultrasound-based neurotechnology platform capable of functioning at brain-wide, region-specific, circuit-specific, or neuron-specific scales. By offering a unified technological approach with scale-optimized transducers for each neural level, the present disclosure reduces procedural complexity, lowers costs, and minimizes invasiveness compared to the current practice of employing multiple disparate technologies.

The section headings used herein are for organizational purposes only and are not to be construed as limiting the subject matter described.

### Definitions

Unless otherwise defined, all of the technical terms used herein have the same meaning as commonly understood by one of ordinary skill in the art in the field to which this disclosure belongs.

As used in this specification and the appended claims, the singular forms “a,” “an,” and “the” include plural references unless the context clearly dictates otherwise. Any reference to “or” herein is intended to encompass “and/or” unless otherwise stated. “About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain’), are inclusive or open-ended and do not exclude additional, un-recited additives, components, integers, elements, or method steps.

As used herein, ordinal terms such as “first,” “second,” “third,” etc., in the claims to modify a claim element does not by itself connote any priority, precedence, or order of one claim element over another, or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

### Scale-Optimized Ultrasound Transducers and Systems Thereof

Disclosed herein is an ultrasound transducer configured to emit ultrasound waves, or receive the ultrasound waves from one or more portions of a nervous system from a subject comprising a sono-enhancing contrast agent, the ultrasound transducer comprising: a sonolucent window; an ultrasonic transducer array, wherein an element in the ultrasonic transducer is 20 to 500 microns wide, and the ultrasonic transducer array comprises at least 100 elements; a control circuit configured to receive or execute instructions for activating a modulating mode or a monitoring mode for the ultrasound transducer; and a processing circuit configured to transform the received ultrasound waves into one or more neural data structures. The ultrasound transducer can alternatively comprise an ultrasonic transducer array that is configured to receive or execute instructions for modulating the one or more portions of the nervous system from the subject, or an ultrasonic transducer array that is configured to receive or execute instructions for monitoring the one or more portions of the nervous system from the subject. The ultrasound transducer can be scale-optimized for monitoring or modulating a whole brain, a brain region or a neural circuit, or a neural circuit or a single neuron. Scale-optimizing the ultrasound transducer for a whole brain can include the use of ultrasonic transducer arrays with element widths between 100 to 500 microns, and circuitry configured to emit 1.0 to 5.0 MHz ultrasound waves via the ultrasonic transducer arrays. Scale-optimizing the ultrasound transducer for a brain region or a neural circuit can include the use of ultrasonic transducer arrays with element widths between 50 -250 microns, and circuitry configured to emit 5.0 to 15 MHz ultrasound waves via the ultrasonic transducer arrays. Scale-optimizing the ultrasound transducer for a brain or a neural circuit can include the use of ultrasonic transducer arrays with element widths between 20-50 microns, and circuitry configured to emit 15 to 100 MHz ultrasound waves via the ultrasonic transducer arrays.

The one or more portions of the nervous system from the subject can comprise a whole brain, a brain region, a neural circuit, or a single neuron. In any of the embodiments herein, the one or more portions of the nervous system from the subject can comprise a dorsolateral prefrontal cortex, frontopolar cortex, anterior cingulate cortex, inferotemporal cortex, visual cortex, auditory cortex, somatosensory cortex, posterior parietal cortex, temporoparietal junction, nucleus accumbens, ventral tegmental area, basal ganglia, orbitofrontal cortex, amygdala, insula, ventromedial prefrontal cortex, hippocampus, entorhinal cortex, parahippcampal gyrus, locus coerulus, reticular formation, ventral striatum, anterior insula, Broca's area, Wernicke's area, medial prefrontal cortex, temporoparietal junction, anterior insula, posterior insula, supplementary motor area, pre-supplementary motor area, or a combination thereof. The one or more portions of the nervous system from the subject can comprise regions involved in executive function, such as the dorsolateral prefrontal cortex, the frontopolar cortex, or the anterior cingular cortex; regions involved in perception and recognition, such as the inferotemporal cortex, the visual cortex, the auditory cortex, or the somatosensory cortex; regions involved in spatial awareness and attention, such as the posterior parietal cortex, or the temporoparietal junction; regions involved in reward processing and motivation, such as the nucleus accumbens, the ventral tegmental area, the basal ganglia, or the orbitofrontal cortex; regions involved in emotional processing, such as the amygdala, the insula, or the ventromedial prefrontal cortex; regions involved in memory, such as the hippocampus, the entorhinal cortex, or the parahippocampal gyrus; regions involved in arousal or attention, such as the locus coerulus, or the reticulus formation; regions involved in salience or valuation, such as the ventral striatum or the anterior insula; regions involved in language processing, such as the Broca's area, or the Wernicke's area; regions involved in self-reference or theory of mind, such as the medial prefrontal cortex, or the temporoparietal junction; regions involved in interoception, such as the anterior insula or the posterior insula; regions involved in cognitive control, such as the supplementary motor area or the presupplementary motor area; or a combination thereof. The whole brain of the subject in relation to the ultrasound transducer, methods, or systems, described herein need not require the monitoring or modulating of the literal whole brain. For example, the ultrasound transducer optimized for the monitoring or the modulating of the whole brain need not monitor or modulate the whole brain, in its exact entirety, of the subject. The whole brain, as used herein, can refer to approximately the whole brain.. Furthermore, the ultrasound transducer optimized for the monitoring or the modulating of the whole brain can be used to monitor or modulate the nervous system across any scope. The whole brain-optimized ultrasound transducer, however, can be understood to be optimized for the use of monitoring or modulating the whole brain or approximately the whole brain. The optimizations that empower the whole brain-optimized ultrasound transducer as described herein can also allow for the whole brain-optimized ultrasound transducer to provide sufficient spatial or temporal resolution for monitoring or modulating other macroscopic scopes of the brain, e.g., when the spatial scope is a priority over the granularity of the spatial resolution during the ultrasound-based monitoring or modulating. In general, the optimizations regarding the ultrasound transducers described herein are not limited by their intended use. The optimizations regarding the ultrasound transducers as described herein need not prohibit the use of the ultrasound transducers for a regime outside the scale and application for which they are optimized. The optimized neural regimes that correspond to any of the scale-optimized ultrasound transducers as described herein, however, provide technical advantages over traditional neuroscientific instrumentation, with respect to monitoring or modulating the optimized neural regime in question.

The one or more neural data structures can correspond to an imaging volume of at most 0.2 cm³, 0.6 cm³, 12 cm³, 100 cm³, 700 cm³ or 1000 cm³. A sub-volume within the imaging volume can be imaged or analyzed, wherein a length of the sub-volume can be as small as the optical resolution of the scale-optimized ultrasound transducer. In any of the embodiments herein, the one or more neural data structures can comprise imaging data at an optical resolution. In some embodiments, the optical resolution can correspond to approximately a width of the element in the ultrasonic transducer array. The optical resolution can correspond to approximately the width of the element in the ultrasonic transducer array, based on an approximately one-to-one ratio. The optical resolution can be at least 20 microns, 25 microns, 50 microns, 100 microns, 150 microns, 200 microns, 250 microns, 300 microns, or 500 microns. Optical resolution can be understood to refer to the spatial resolution of data, e.g., one or more neural data structures associated with the ultrasound transducers, or the spatial resolution deriving from the ultrasound transducer itself (e.g., the spatial resolution related to the optical components of the ultrasound transducer itself). That is, the optical resolution can be understood as the minimum distinguishable distance between two adjacent features or structures in the neural tissue, as obtained via ultrasound monitoring or ultrasound modulating. The optical resolution need not be limited to the context of light, e.g., to visible light, but can extend generally to waveforms, e.g., to waveforms that can be expressed in terms of a point spread function. Optical resolution can refer to the wavelength limited resolution, e.g., diffraction limited resolution, of the point spread function. Optical resolution in the context of ultrasound waves can refer to the wavelength limited resolution, e.g., diffraction limited resolution, of the acoustic point spread function. The acoustic point spread function can be understood to be the impulse response of a focused optical imaging system, such as that of an ultrasound system or an ultrasound transducer.

The one or more neural data structures can comprise precursor imaging data at a computational superresolution. The computational superresolution can correspond to less than the width of the element in the ultrasonic transducer array. The computational superresolution can correspond to approximately the width of the element in the ultrasonic transducer array, based on an approximately multi-to-one ratio. The one or more neural data structures can comprise imaging data at an imaging depth. In some embodiments, the imaging depth can be at least 0.04 cm, 0.1 cm, 0.2 cm, 0.4 cm, 0.5 cm, 2.0 cm, 4.0 cm, 6.0 cm, or 8.0 cm. The ultrasound waves can comprise a frequency of at most 100 MHz, 60 MHz, 15 MHz, 7.5 MHz, 5.0 MHz, 2.5 MHz, or 1.0 MHz. The optical resolution can correspond to approximately the frequency. The computational superresolution can correspond to approximately the frequency.

The one or more neural data structures can comprise hemodynamic data. The embodiments herein, the one or more neural data structures can comprise neural activity data. The one or more neural data structures can comprise neural anatomy data.

The one or more neural data structures can be any data structure that can be represented as one or more arrays or collections of data points. For example, the one or more neural data structures can comprise tensors, where the tensors can relate to ultrasound patterns corresponding to the nervous system of the subject. These data structures may include vectors (one-dimensional arrays), traditional matrices (two-dimensional arrays), three-dimensional arrays, multi-dimensional tensors, sparse matrices, compressed representations, hierarchical data structures, graph-based representations, time-series arrays, frequency-domain representations, wavelet decompositions, statistical distributions, probability density functions, feature maps, embedding spaces, dimensionality-reduced representations, manifolds, latent variable spaces, point clouds, voxel grids, octrees, mesh structures, or any combination thereof. The neural data structures may further represent different types of neural information, including spatial coordinates, temporal dynamics, frequency characteristics, phase relationships, correlation structures, connectivity patterns, network topologies, activation patterns, inhibition patterns, hemodynamic responses, metabolic activities, electrical potentials, receptor distributions, neurotransmitter concentrations, structural features, tissue properties, vascular arrangements, white matter tracts, or any other measurable or derivable neural properties. The neural data structures may be pre-processed, filtered, normalized, standardized, transformed, compressed, encrypted, error-corrected, noise-reduced, motion-compensated, artifact-removed, temporally or spatially aligned, interpolated, extrapolated, segmented, clustered, classified, or otherwise algorithmically manipulated to enhance their utility for subsequent analysis, visualization, or modulation purposes. Implementation of these data structures may use any suitable computational representation including fixed-point or floating-point numeric formats, binary encodings, quantized representations, or specialized data types optimized for neural data processing or machine learning operations. The one or more neural data structures can encompass spatial or temporal attributes, in addition to channel data, such as optical channel data. The one or more neural data structures can be dense neural data structures, or sparse neural data structures. The sparse neural data structures can store only voxels containing relevant neural information using coordinate-value pairs, and can be understood to be a sparse matrix or a sparse tensor. Sparse neural data structures can include sparse coordinate formats, indexed voxel collections, or point cloud representations. The one or more neural data structures can include radiofrequency (RF) data, in-phase quadrature (IQ) data, processed RF data, or processed IQ data. The one or more neural data structures can comprise data structures derived from ultrasound waves reflected, e.g., scattered, from the nervous system of the subject and received by the ultrasound transducer.

The element in the ultrasonic transducer array can be circular, elliptical, square, rectangular, hexagonal, regular, or irregular, in shape. The width of an element in the ultrasonic transducer array can refer to a diameter, a cross-length, a length, or some other dimension relevant to the geometry of the ultrasonic transducer array element.

The ultrasonic transducer array can be a capacitive micromachined ultrasonic transducer (CMUT) array or a piezoelectric micromachined ultrasonic transducer (PMUT) array. The CMUT array can comprise a flat membrane CMUT, a curved membrane CMUT, a dual-electrode CMUT, a collapsed-mode CMUT, a wafer-bonded CMUT, a surface-micromachined CMUT, a CMUT-on-CMOS integrated device, a piston-shaped CMUT, an annular array CMUT, or a flexible CMUT. In some embodiments, the PMUT array can comprise a unimorph PMUT, a bimorph PMUT, a dome-shaped PMUT, a suspended membrane PMUT, an aluminum nitride PMUT, a lead zirconate titanate PMUT, a scandium-doped aluminum nitride PMUT, a lithium niobate PMUT, a polyvinylidene fluoride PMUT, a thin-film PMUT, or a flexible PMUT.

The ultrasound transducer can be an implantable ultrasound transducer. The ultrasound transducer can be a non-implantable ultrasound transducer. The non-implantable ultrasound transducer can comprise a wearable ultrasound transducer.

The sono-enhancing contrast agent can comprise a biological sono-enhancing contrast agent, a polymeric sono-enhancing contrast agent, or a pharmaceutical sono-enhancing contrast agent. The biological sono-enhancing contrast agent can comprise a genetically-encoded sono-enhancing contrast agent. The genetically-encoded sono-enhancing contrast agent can comprise an ultrasonic reporter of calcium, an ultrasonic reporter of voltage, or an ultrasonic reporter of a neurotransmitter, e.g., glutamate, GABA, dopamine, or acetylcholine. The ultrasonic reporter of calcium can be based on calcium-gated binding proteins, such as calmodulin and calmodulin-binding proteins. The sono-enhancing contrast agent can also leverage the physical properties of gas vesicles. For example, the altering of gas vesicles' conformations, e.g., “buckling”, upon stimulation by ultrasound waves of a specific frequency range, can reflect back sono-enhanced ultrasound waves towards one or more ultrasound transducers. Accordingly, the sono-enhancing contrast agent can comprise gas vesicles. The gas vesicles of the sono-enhancing contrast agent can be modified to buckle at different susceptibilities, e.g., at different strengths (e.g., amplitudes) of ultrasound waves. For example, the gas vesicle's protein shell may or may not comprise GvpC or GvpA proteins. In addition, the sono-enhancing contrast agent can be expressed in accordance with different temporal and/or spatial patterns in the subject. For example, for temporal gating, the sono-enhancing contrast agent can be activated, e.g., via an inducible promoter. For spatial gating, the sono-enhancing contrast agent can be localized, e.g., via a tissue-specific promoter. Both temporal and spatial gating can be used to impose further conditions that control the activation, and thus utility, of the sono-enhancing contrast agent. Accordingly, the condition on calcium for the activation of ultrasonic reporters of calcium can be understood to be a temporal-spatial gating on the sono-enhancing contrast agent. The sono-enhancing contrast agent can comprise a polymeric sono-enhancing contrast agent. The polymeric sono-enhancing contrast agent can comprise a silica-based particle. The silica-based particle can include a solid silica core, an intermediate film of polymethacrylic acid (PMA) that can be cross-linked or pH-responsive, and an outer porous organosilica shell. The polymeric sono-enhancing contrast agent can indicate the neural activity of a subject by exploiting the intracellular or extracellular pH changes associated with neural activity in a subject. For example, the carboxyl group of the polymeric contrast agent, e.g., the PMA-based silica particle, may protonate under acidic pH, which can cause the film of PMA to collapse, and thereby alter the acoustic properties of the polymeric sono-enhancing contrast agent. In contrast, at neutral pH, the carboxyl groups of the PMA-based silica particle may deprotonated, which can cause the carboxyl groups to repel each other, and thereby cause the film of PMA to swell with water. The polymeric sono-enhancing contrast agent can be further engineered to respond directly to physiological indicators of neural activity, such as cellular changes in voltage or calcium in neurons. The polymeric sono-enhancing contrast agent can be delivered into the nervous system, e.g., the brain, of the subject via crossing the blood-brain barrier of the subject. The pharmaceutical sono-enhancing contrast agent can include a small molecule, e.g., a drug. The pharmaceutical sono-enhancing contrast agent can be functionally coupled to a physiological indicator of neural activity, such as intracellular or extracellular calcium changes, intracellular or extracellular voltage changes, or intracellular or extracellular changes in a neurotransmitter. The pharmaceutical contrast agent can be delivered into the nervous system, e.g., the brain, of the subject via crossing the blood-brain barrier of the subject.

The sono-enhancing contrast agent may be expressed in a pattern that is broader than the neural scale for which the scale-optimized ultrasound transducer is optimized. That is, for an ultrasound transducer optimized for brain region-specific imaging, the pattern of the sono-enhancing contrast agent may be ubiquitous across a broader region of the brain, but such a broad expression pattern would not necessarily impede the target imaging or modulating scale of the ultrasound transducer, because some of the sono-enhancing contrast agent-expressing cells may be outside the field of view of the one or more ultrasound transducers, and/or because the sono-enhancing contrast agent may not be activated due to spatial or temporal gatings.

The subject can comprise a human subject, or a non-human primate, mammal, vertebrate, or model organism used in neuroscience research. The subject can be of any age, developmental stage, sex, or genetic background. The subject can have, or can be suspected of having, a neural dysfunction, condition, disorder, disease, injury, abnormality, or altered neural state. The neural dysfunction can comprise a motor dysfunction, a sensory dysfunction, a cognitive dysfunction, a neurological condition, a neurodevelopmental disorder, a neurodegenerative disease, a neuroimmunological disorder, a neurometabolic condition, a neurovascular disorder, a neuroendocrine dysfunction, or a neuropsychiatric dysfunction. The motor dysfunction can comprise a spinal cord injury, traumatic brain injury, amyotrophic lateral sclerosis, multiple sclerosis, cerebral palsy, locked-in syndrome, stroke, Parkinson's disease, Huntington's disease, dystonia, essential tremor, ataxia, apraxia, dyskinesia, tic disorders, myoclonus, restless leg syndrome, spasticity, muscle weakness, paralysis, hemiplegia, quadriplegia, movement disorders, or motor neuron diseases. The sensory dysfunction can comprise blindness, deafness, anosmia, hyposmia, tinnitus, hyperacusis, vertigo, dysgeusia, analgesia, hyperalgesia, allodynia, paresthesia, anesthesia, tactile agnosia, proprioceptive disorders, epilepsy, visual processing disorders, auditory processing disorders, a loss of chemosensation, or a loss of somatosensation. The cognitive dysfunction can comprise memory disorders, attention deficits, executive function impairments, language disorders, visuospatial deficits, processing speed impairments, learning disabilities, dementia, mild cognitive impairment, Alzheimer's disease, frontotemporal dementia, Lewy body dementia, vascular dementia, amnesia, aphasia, agnosia, or cognitive sequelae of neurological conditions. The neuropsychiatric dysfunction can comprise clinical depression, bipolar disorder, schizophrenia, schizoaffective disorder, psychosis, anxiety disorders, obsessive-compulsive disorder, post-traumatic stress disorder, addiction, substance use disorders, alcohol use disorder, opioid use disorder, personality disorders, eating disorders, anorexia nervosa, bulimia nervosa, binge eating disorder, sleep disorders, insomnia, narcolepsy, sleep apnea, circadian rhythm disorders, impulse control disorders, conduct disorders, autism spectrum disorders, attention deficit hyperactivity disorder, or any condition affecting mental health with neurobiological underpinnings. The neural dysfunction may be acute, chronic, progressive, remitting, relapsing, congenital, acquired, genetic, idiopathic, primary, secondary, or of mixed etiology. The subject may have comorbid conditions, multiple interacting neural dysfunctions, or overlapping symptomatology. The subject may be at risk for developing a neural dysfunction based on genetic factors, environmental exposures, prior medical history, biomarkers, or prodromal symptoms. The subject may be experiencing subclinical manifestations, early-stage progression, advanced disease, recovery phases, or compensatory adaptations related to neural dysfunction. The subject may be treatment-naïve, undergoing active treatment, treatment-resistant, or in remission.

The ultrasound transducer can provide a neural augmenting to the subject. The neural augmenting can comprise sensorimotor integrating, sensory augmenting, integrating to a virtual reality, integrating to an augmented reality, modulating a brain state, or integrating to an artificial intelligence.

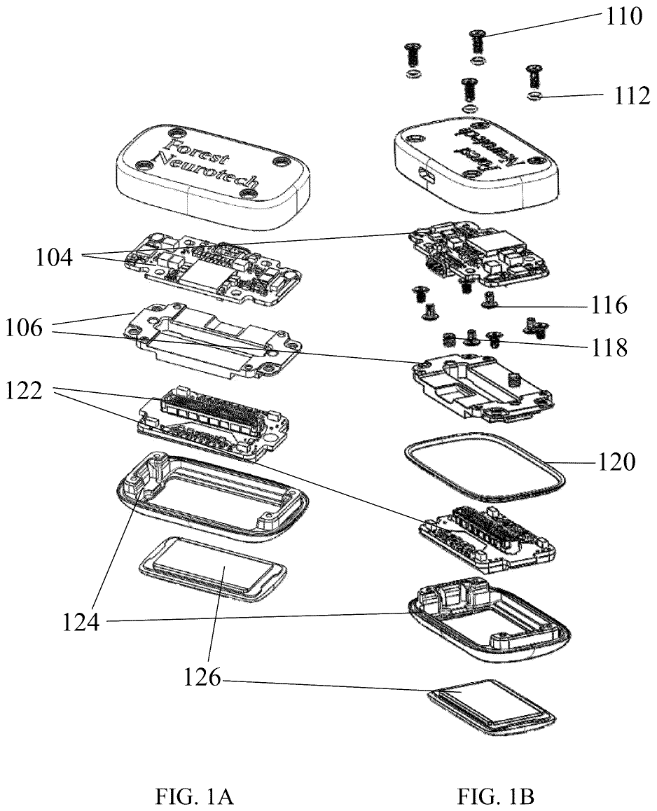

FIGS. 1A and 1B provide exploded view renderings of an ultrasound transducer, e.g., a wearable ultrasound transducer. The parts depicted for the wearable ultrasound transducer in FIGS. 1A and 1B can be applicable for ultrasound transducers configured for monitoring and/or modulating the nervous system of a subject. As shown in FIGS. 1A and 1B, the implantable ultrasound transducer can comprise one or more of: an upper housing 102, which can be made of hard anodized (type III) 6061-alloy aluminum; an FPGA circuit board 104; a heat sink 106 (e.g., made of 6061-alloy aluminum); a lower housing assembly 108 comprising an ultrasound transducer; wafer head screws 110 (e.g., M2×5 mm wafer head screws made of 316 stainless steel); an O-ring 112 (e.g., made of FKM rubber); an M2 washer 114 (e.g., made of PTFE or Nylon 6); wafer head screws 116 (e.g., M2×3 mm wafer head screws made of 316 stainless steel); a screw set 118 (e.g., an M3×3 mm set screw comprising a 316 stainless steel); and a housing seal 120 (e.g., made of an elastomer). FIG. 1B further depicts a populated circuit board comprising a MEMS array 122, contained within the lower housing assembly 108, a chassis component 124 of the lower housing assembly 108, and a sonolucent window 126 comprising a biocompatible material that permits minimal acoustic impedance and minimal ultrasound attenuation, such as PMMA, PEEK, PCTFE, PTFE, UHMWPE, PET, LDPE, PEBAX, and/or HDPE. The MEMS array of the chassis component 122 is an ultrasonic transducer array, and can be, for example, a CMUT array or a PMUT array. The element widths of the array depicted for the populated circuit board comprising the MEMS array 122 can be manufactured such that each element width is optimized for the monitoring or modulating at a specific neural scale. An element width of 100-500 microns can be suitable for an ultrasound array optimized for brain-wide monitoring or modulating. An element width of 50 -250 microns can be suitable for an ultrasound array optimized for brain region-level or neural circuit-level monitoring or modulating. An element width of 20-50 microns can be suitable for an ultrasound array optimized for neural circuit-level or single neuron-level monitoring or modulating. The supporting circuit board, as illustrated in the populated circuit board comprising the MEMS array 122 can be engineered to support the MEMS array with the target element width.

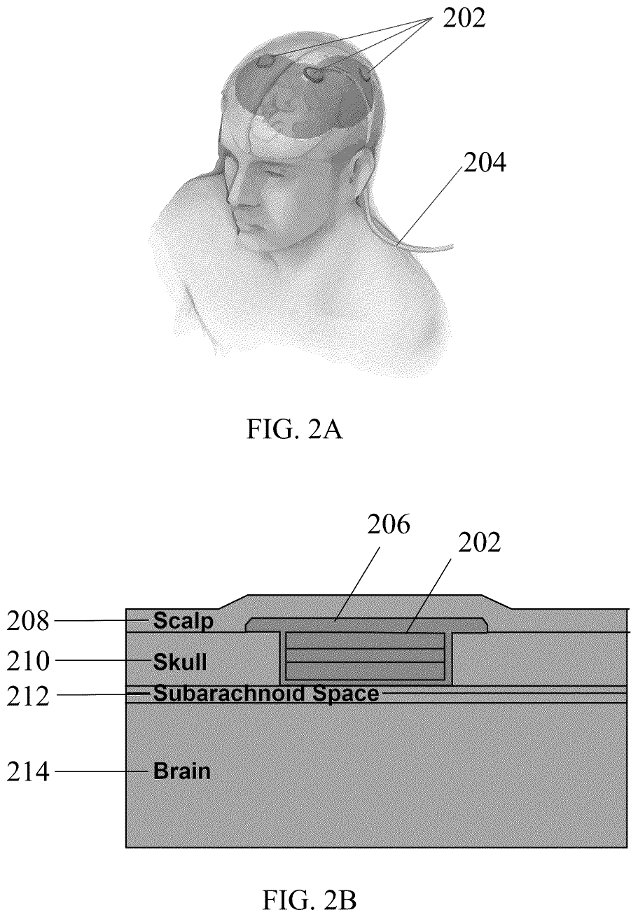

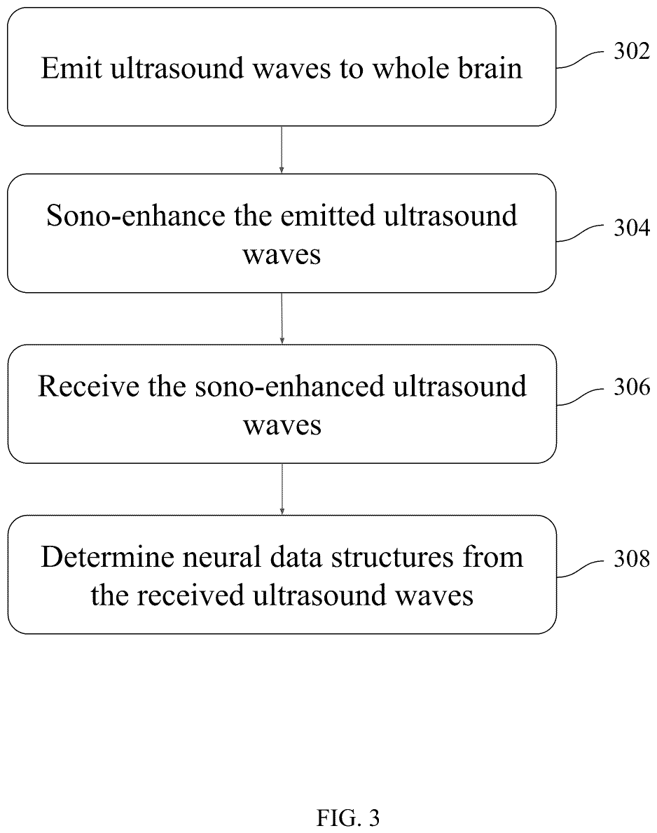

The ultrasound transducers and their associated systems and methods as described herein can comprise an ultrasound-based monitoring and modulating device enclosed by a rigid housing designed to mount to the skull of a living human subject, as shown in FIGS. 2A and 2B. That is, the ultrasound transducer can be an implantable ultrasound transducer. As shown in both FIGS. 2A and 2B, the implantable ultrasound transducer 202 may be configured to fit within a hole in the skull (e.g., a burr hole) and sit in contact with the dura mater (completely outside the brain parenchyma). In some implementations, the implantable ultrasound transducer 202 can be positioned to be in contact with a soft tissue of a subject, such as, but not limited to, muscles, fat, blood vessels, nerves, tendons, or any combination thereof. Due to its use within the body, the implantable ultrasound transducer 202 can be hermetically sealed and sterilized within a housing 206. The implantable ultrasound transducer 202 can be connected to a cable 204, which can provide power and/or data to and/or from the implantable ultrasound transducer 202. The cable 204 can connect the implantable ultrasound transducer 202 to a peripheral controller and/or another implantable ultrasound transducer 202. The implantable ultrasound transducer 202 can be implanted in a minimally invasive way, such that each implantable ultrasound transducer can be installed in a small burr hole below the scalp 208 of the subject, such that the burr hole is in the skull 210 of the subject. The positioning of the implantable ultrasound transducer 202 may be such that the implantable ultrasound transducer 202 sits above the subarachnoid space 212, and does not require penetration into the brain tissue 214.

### Methods of Modulating or Monitoring One or More Portions of a Nervous System With a Sono-Enhancing Contrast Agent

Disclosed herein is a method of modulating or monitoring a one or more portions of a nervous system of a subject comprising a sono-enhancing contrast agent, comprising: emitting ultrasound waves to the whole brain, from an ultrasound transducer corresponding to the ultrasound transducer of any of the embodiments herein; sono-enhancing the emitted ultrasound waves, via the sono-enhancing contrast agent in the subject, to form sono-enhanced ultrasound waves; receiving the sono-enhanced ultrasound waves from the subject, by the ultrasound transducer; and determining one or more neural data structures, using one or more processors, based on the received sono-enhanced ultrasound waves. The one or more portions of the nervous system can include a whole brain, one or more brain regions, one or more neural circuits, or one or more single neurons.

At 302 in FIG. 3, ultrasound waves are emitted to a whole brain of a subject, from an ultrasound transducer corresponding to the ultrasound transducer of any of the embodiments herein. The element in the ultrasonic transducer array can be circular, elliptical, square, rectangular, hexagonal, regular, or irregular, in shape. The width of an element in the ultrasonic transducer array can refer to a diameter, a cross-length, a length, or some other dimension relevant to the geometry of the ultrasonic transducer array element.

The ultrasonic transducer array can be a capacitive micromachined ultrasonic transducer (CMUT) array or a piezoelectric micromachined ultrasonic transducer (PMUT) array. The CMUT array can comprise a flat membrane CMUT, a curved membrane CMUT, a dual-electrode CMUT, a collapsed-mode CMUT, a wafer-bonded CMUT, a surface-micromachined CMUT, a CMUT-on-CMOS integrated device, a piston-shaped CMUT, an annular array CMUT, or a flexible CMUT. In some embodiments, the PMUT array can comprise a unimorph PMUT, a bimorph PMUT, a dome-shaped PMUT, a suspended membrane PMUT, an aluminum nitride PMUT, a lead zirconate titanate PMUT, a scandium-doped aluminum nitride PMUT, a lithium niobate PMUT, a polyvinylidene fluoride PMUT, a thin-film PMUT, or a flexible PMUT.

At 304 in FIG. 3, the emitted ultrasound waves are sono-enhanced, via the sono-enhancing contrast agent in the subject, to form sono-enhanced ultrasound waves. The sono-enhancing contrast agent can comprise a biological sono-enhancing contrast agent, a polymeric sono-enhancing contrast agent, or a pharmaceutical sono-enhancing contrast agent. The biological sono-enhancing contrast agent can comprise a genetically-encoded sono-enhancing contrast agent. The genetically-encoded sono-enhancing contrast agent can comprise an ultrasonic reporter of calcium, an ultrasonic reporter of voltage, or an ultrasonic reporter of a neurotransmitter, e.g., glutamate, GABA, dopamine, or acetylcholine. The ultrasonic reporter of calcium can be based on calcium-gated binding proteins, such as calmodulin and calmodulin-binding proteins. The sono-enhancing contrast agent can also leverage the physical properties of gas vesicles. For example, the altering of gas vesicles' conformations, e.g., “buckling”, upon stimulation by ultrasound waves of a specific frequency range, can reflect back sono-enhanced ultrasound waves towards one or more ultrasound transducers. Accordingly, the sono-enhancing contrast agent can comprise gas vesicles. The gas vesicles of the sono-enhancing contrast agent can be modified to buckle at different susceptibilities, e.g., at different strengths (e.g., amplitudes) of ultrasound waves. For example, the gas vesicle's protein shell may or may not comprise GvpC or GvpA proteins. In addition, the sono-enhancing contrast agent can be expressed in accordance with different temporal and/or spatial patterns in the subject. For example, for temporal gating, the sono-enhancing contrast agent can be activated, e.g., via an inducible promoter. For spatial gating, the sono-enhancing contrast agent can be localized, e.g., via a tissue-specific promoter. Both temporal and spatial gating can be used to impose further conditions that control the activation, and thus utility, of the sono-enhancing contrast agent. Accordingly, the condition on calcium for the activation of ultrasonic reporters of calcium can be understood to be a temporal-spatial gating on the sono-enhancing contrast agent. The sono-enhancing contrast agent can comprise a polymeric sono-enhancing contrast agent. The polymeric sono-enhancing contrast agent can comprise a silica-based particle. The silica-based particle can include a solid silica core, an intermediate film of polymethacrylic acid (PMA) that can be cross-linked or pH-responsive, and an outer porous organosilica shell. The polymeric sono-enhancing contrast agent can indicate the neural activity of a subject by exploiting the intracellular or extracellular pH changes associated with neural activity in a subject. For example, the carboxyl group of the polymeric contrast agent, e.g., the PMA-based silica particle, may protonate under acidic pH, which can cause the film of PMA to collapse, and thereby alter the acoustic properties of the polymeric sono-enhancing contrast agent. In contrast, at neutral pH, the carboxyl groups of the PMA-based silica particle may deprotonated, which can cause the carboxyl groups to repel each other, and thereby cause the film of PMA to swell with water. The polymeric sono-enhancing contrast agent can be further engineered to respond directly to physiological indicators of neural activity, such as cellular changes in voltage or calcium in neurons. The polymeric sono-enhancing contrast agent can be delivered into the nervous system, e.g., the brain, of the subject via crossing the blood-brain barrier of the subject. The pharmaceutical sono-enhancing contrast agent can include a small molecule, e.g., a drug. The pharmaceutical sono-enhancing contrast agent can be functionally coupled to a physiological indicator of neural activity, such as intracellular or extracellular calcium changes, intracellular or extracellular voltage changes, or intracellular or extracellular changes in a neurotransmitter. The pharmaceutical contrast agent can be delivered into the nervous system, e.g., the brain, of the subject via crossing the blood-brain barrier of the subject.

The sono-enhancing contrast agent may be expressed in a pattern that is broader than the neural scale for which the scale-optimized ultrasound transducer is optimized. That is, for an ultrasound transducer optimized for brain region-specific imaging, the pattern of the sono-enhancing contrast agent may be ubiquitous across the brain, but such a broad expression pattern would not necessarily impede the target imaging or modulating scale of the ultrasound transducer, because some of the sono-enhancing contrast agent-expressing cells may be outside the field of view of the one or more ultrasound transducers, and/or because the sono-enhancing contrast agent may not be activated due to spatial or temporal gatings.

At 306 in FIG. 3, the sono-enhanced ultrasound waves from the subject are received, by the ultrasound transducer.

At 308 in FIG. 3, one or more neural data structures are determined, using one or more processors, based on the received sono-enhanced ultrasound waves. The one or more neural data structures can be any data structure that can be represented as one or more arrays or collections of data points. For example, the one or more neural data structures can comprise tensors, where the tensors can relate to ultrasound patterns corresponding to the nervous system of the subject. These data structures may include vectors (one-dimensional arrays), traditional matrices (two-dimensional arrays), three-dimensional arrays, multi-dimensional tensors, sparse matrices, compressed representations, hierarchical data structures, graph-based representations, time-series arrays, frequency-domain representations, wavelet decompositions, statistical distributions, probability density functions, feature maps, embedding spaces, dimensionality-reduced representations, manifolds, latent variable spaces, point clouds, voxel grids, octrees, mesh structures, or any combination thereof. The neural data structures may further represent different types of neural information, including spatial coordinates, temporal dynamics, frequency characteristics, phase relationships, correlation structures, connectivity patterns, network topologies, activation patterns, inhibition patterns, hemodynamic responses, metabolic activities, electrical potentials, receptor distributions, neurotransmitter concentrations, structural features, tissue properties, vascular arrangements, white matter tracts, or any other measurable or derivable neural properties. The neural data structures may be pre-processed, filtered, normalized, standardized, transformed, compressed, encrypted, error-corrected, noise-reduced, motion-compensated, artifact-removed, temporally or spatially aligned, interpolated, extrapolated, segmented, clustered, classified, or otherwise algorithmically manipulated to enhance their utility for subsequent analysis, visualization, or modulation purposes. Implementation of these data structures may use any suitable computational representation including fixed-point or floating-point numeric formats, binary encodings, quantized representations, or specialized data types optimized for neural data processing or machine learning operations. The one or more neural data structures can encompass spatial or temporal attributes, in addition to channel data, such as optical channel data. The one or more neural data structures can be dense neural data structures, or sparse neural data structures. The sparse neural data structures can store only voxels containing relevant neural information using coordinate-value pairs, and can be understood to be a sparse matrix or a sparse tensor. Sparse neural data structures can include sparse coordinate formats, indexed voxel collections, or point cloud representations. The one or more neural data structures can include radiofrequency (RF) data, in-phase quadrature (IQ) data, processed RF data, or processed IQ data. The one or more neural data structures can comprise data structures derived from ultrasound waves reflected, e.g., scattered, from the nervous system of the subject and received by the ultrasound transducer.

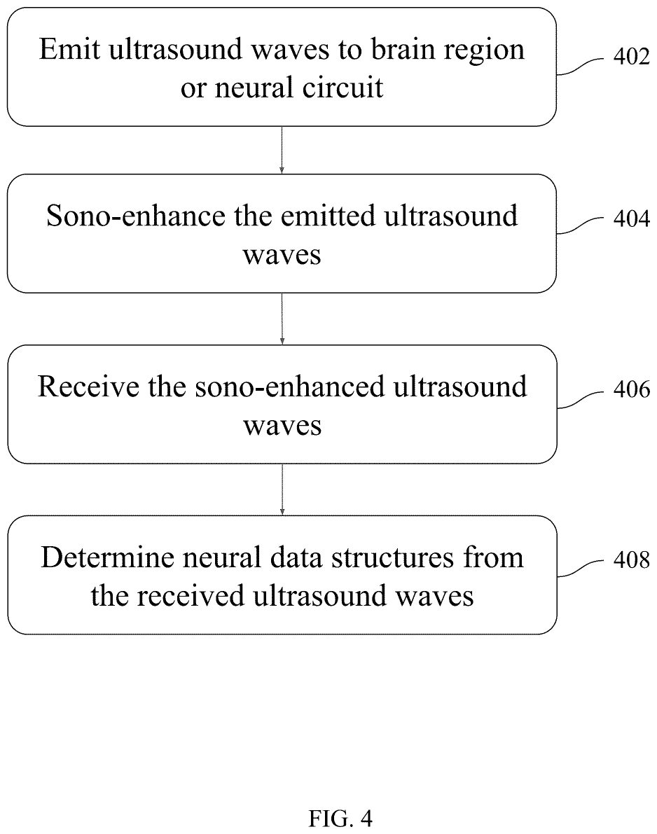

At 402 in FIG. 4, ultrasound waves are emitted to a brain region or a neural circuit of a subject, from an ultrasound transducer corresponding to the ultrasound transducer of any of the embodiments herein. The ultrasonic transducer array can be a capacitive micromachined ultrasonic transducer (CMUT) array or a piezoelectric micromachined ultrasonic transducer (PMUT) array. The CMUT array can comprise a flat membrane CMUT, a curved membrane CMUT, a dual-electrode CMUT, a collapsed-mode CMUT, a wafer-bonded CMUT, a surface-micromachined CMUT, a CMUT-on-CMOS integrated device, a piston-shaped CMUT, an annular array CMUT, or a flexible CMUT. In some embodiments, the PMUT array can comprise a unimorph PMUT, a bimorph PMUT, a dome-shaped PMUT, a suspended membrane PMUT, an aluminum nitride PMUT, a lead zirconate titanate PMUT, a scandium-doped aluminum nitride PMUT, a lithium niobate PMUT, a polyvinylidene fluoride PMUT, a thin-film PMUT, or a flexible PMUT.

The control circuit configured to receive or execute instructions for activating the modulating mode or the monitoring mode can refer to any hardware, software, firmware, or combination thereof capable of managing, directing, or regulating the operation of the ultrasound transducer. The control circuit may include one or more microprocessors, microcontrollers, field-programmable gate arrays (FPGAs), application-specific integrated circuits (ASICs), digital signal processors (DSPs), programmable logic devices (PLDs), complex programmable logic devices (CPLDs), system-on-chip (SoC) devices, embedded controllers, state machines, sequencers, logic circuits, analog circuits, digital circuits, mixed-signal circuits, timing circuits, counters, registers, memory devices, buses, input/output (I/O) interfaces, signal conditioning circuitry, amplifiers, filters, oscillators, phase-locked loops (PLLs), clock generators, power management circuits, wireless communication modules, wired communication interfaces, multiplexers, demultiplexers, analog-to-digital converters (ADCs), digital-to-analog converters (DACs), voltage regulators, current sources, switching elements, or any combination thereof. The control circuit may be centralized in a single location or distributed across multiple components or subsystems. The control circuit may be implemented on one or more printed circuit boards (PCBs), integrated circuits, multi-chip modules, flexible circuits, rigid-flex circuits, ceramic substrates, or other suitable substrates. The control circuit may execute firmware, operating systems, real-time operating systems, device drivers, middleware, application software, neural network models, machine learning algorithms, or signal processing routines. The control circuit may be configured to receive instructions from external sources including, but not limited to, wired connections, wireless connections, optical connections, radiofrequency identification (RFID), near-field communication (NFC), Bluetooth, Bluetooth Low Energy, WiFi, WiFi HaLow, cellular networks, satellite communications, infrared, ultrasonic communication, optical data communication, Light Fidelity (Li-Fi), ultra-wideband, impulse-radio ultra-wideband, ultrasonic data transmission, wireless chip-to-chip, manual inputs, millimeter wave technologies, or a combination thereof. The control circuit may further include error detection and correction mechanisms, self-diagnostic capabilities, calibration functionalities, fault tolerance features, redundancy systems, and security measures to protect against unauthorized access or interference. The control circuit may operate in open-loop or closed-loop configurations, with or without feedback mechanisms for maintaining desired operational parameters.

The processing circuit configured to transform the received ultrasound waves can refer to any hardware, software, firmware, or combination thereof capable of performing operations, computations, transformations, analyses, or manipulations on data derived from the ultrasound waves. The processing circuit may include, one or more microprocessors, microcontrollers, central processing units (CPUs), graphics processing units (GPUs), tensor processing units (TPUs), digital signal processors (DSPs), field-programmable gate arrays (FPGAs), application-specific integrated circuits (ASICs), system-on-chip (SoC) devices, neuromorphic processors, quantum processors, cloud computing resources, distributed computing systems, parallel processing arrays, vector processors, arithmetic logic units (ALUs), co-processors, accelerators, memory controllers, cache systems, register files, instruction decoders, pipeline architectures, branch predictors, specialized computing elements, programmable logic devices (PLDs), complex programmable logic devices (CPLDs), analog circuits, digital circuits, mixed-signal circuits, or any combination thereof. The processing circuit may implement various signal processing operations including, but not limited to, filtering, amplification, denoising, beamforming, pulse compression, envelope detection, log compression, time gain compensation, dynamic range adjustment, scan conversion, image reconstruction, feature extraction, segmentation, registration, motion compensation, phase detection, frequency analysis, time-domain analysis, spatial-domain analysis, transform-domain processing, statistical analysis, parametric estimation, dimensionality reduction, or data fusion. The processing circuit may employ algorithms including adaptive filters, wavelet transforms, Fourier transforms, compressive sensing, sparse representations, machine learning, deep learning, neural networks, probabilistic models, statistical models, physical models, biomechanical models, inverse problem solving, regularization techniques, optimization methods, classification algorithms, clustering algorithms, pattern recognition, spectral analysis, temporal analysis, spatial analysis, or multiscale analysis. The processing circuit may be implemented on one or more printed circuit boards (PCBs), integrated circuits, multi-chip modules, flexible circuits, rigid-flex circuits, ceramic substrates, or other suitable substrates. The processing circuit may be centralized in a single location or distributed across multiple components or subsystems, and may include cloud-based or edge computing architectures. The processing circuit may further incorporate error handling, parallel processing, pipelined processing, real-time processing, batch processing, high-performance computing techniques, memory management, data storage, caching strategies, or energy optimization methods. The processing circuit may interact with other system components through various interfaces, buses, networks, or communication protocols to receive input data, provide output results, or exchange intermediate information.

At 404 in FIG. 4, the emitted ultrasound waves are sono-enhanced, via the sono-enhancing contrast agent in the subject, to form sono-enhanced ultrasound waves. The sono-enhancing contrast agent can comprise a biological sono-enhancing contrast agent, a polymeric sono-enhancing contrast agent, or a pharmaceutical sono-enhancing contrast agent. The biological sono-enhancing contrast agent can comprise a genetically-encoded sono-enhancing contrast agent. The genetically-encoded sono-enhancing contrast agent can comprise an ultrasonic reporter of calcium, an ultrasonic reporter of voltage, or an ultrasonic reporter of a neurotransmitter, e.g., glutamate, GABA, dopamine, or acetylcholine. The ultrasonic reporter of calcium can be based on calcium-gated binding proteins, such as calmodulin and calmodulin-binding proteins. The sono-enhancing contrast agent can also leverage the physical properties of gas vesicles. For example, the altering of gas vesicles' conformations, e.g., “buckling”, upon stimulation by ultrasound waves of a specific frequency range, can reflect back sono-enhanced ultrasound waves towards one or more ultrasound transducers. Accordingly, the sono-enhancing contrast agent can comprise gas vesicles. The gas vesicles of the sono-enhancing contrast agent can be modified to buckle at different susceptibilities, e.g., at different strengths (e.g., amplitudes) of ultrasound waves. For example, the gas vesicle's protein shell may or may not comprise GvpC or GvpA proteins. In addition, the sono-enhancing contrast agent can be expressed in accordance with different temporal and/or spatial patterns in the subject. For example, for temporal gating, the sono-enhancing contrast agent can be activated, e.g., via an inducible promoter. For spatial gating, the sono-enhancing contrast agent can be localized, e.g., via a tissue-specific promoter. Both temporal and spatial gating can be used to impose further conditions that control the activation, and thus utility, of the sono-enhancing contrast agent. Accordingly, the condition on calcium for the activation of ultrasonic reporters of calcium can be understood to be a temporal-spatial gating on the sono-enhancing contrast agent. The sono-enhancing contrast agent can comprise a polymeric sono-enhancing contrast agent. The polymeric sono-enhancing contrast agent can comprise a silica-based particle. The silica-based particle can include a solid silica core, an intermediate film of polymethacrylic acid (PMA) that can be cross-linked or pH-responsive, and an outer porous organosilica shell. The polymeric sono-enhancing contrast agent can indicate the neural activity of a subject by exploiting the intracellular or extracellular pH changes associated with neural activity in a subject. For example, the carboxyl group of the polymeric contrast agent, e.g., the PMA-based silica particle, may protonate under acidic pH, which can cause the film of PMA to collapse, and thereby alter the acoustic properties of the polymeric sono-enhancing contrast agent. In contrast, at neutral pH, the carboxyl groups of the PMA-based silica particle may deprotonated, which can cause the carboxyl groups to repel each other, and thereby cause the film of PMA to swell with water. The polymeric sono-enhancing contrast agent can be further engineered to respond directly to physiological indicators of neural activity, such as cellular changes in voltage or calcium in neurons. The polymeric sono-enhancing contrast agent can be delivered into the nervous system, e.g., the brain, of the subject via crossing the blood-brain barrier of the subject. The pharmaceutical sono-enhancing contrast agent can include a small molecule, e.g., a drug. The pharmaceutical sono-enhancing contrast agent can be functionally coupled to a physiological indicator of neural activity, such as intracellular or extracellular calcium changes, intracellular or extracellular voltage changes, or intracellular or extracellular changes in a neurotransmitter. The pharmaceutical contrast agent can be delivered into the nervous system, e.g., the brain, of the subject via crossing the blood-brain barrier of the subject.

The sono-enhancing contrast agent may be expressed in a pattern that is broader than the neural scale for which the scale-optimized ultrasound transducer is optimized. That is, for an ultrasound transducer optimized for brain region-specific imaging, the pattern of the sono-enhancing contrast agent may be ubiquitous across the brain, but such a broad expression pattern would not necessarily impede the target imaging or modulating scale of the ultrasound transducer, because some of the sono-enhancing contrast agent-expressing cells may be outside the field of view of the one or more ultrasound transducers, and/or because the sono-enhancing contrast agent may not be activated due to spatial or temporal gatings.

At 406 in FIG. 4, the sono-enhanced ultrasound waves from the subject are received, by the ultrasound transducer.

At 408 in FIG. 4, one or more neural data structures are determined, using one or more processors, based on the received sono-enhanced ultrasound waves. The one or more neural data structures can be any data structure that can be represented as one or more arrays or collections of data points. For example, the one or more neural data structures can comprise tensors, where the tensors can relate to ultrasound patterns corresponding to the nervous system of the subject. These data structures may include vectors (one-dimensional arrays), traditional matrices (two-dimensional arrays), three-dimensional arrays, multi-dimensional tensors, sparse matrices, compressed representations, hierarchical data structures, graph-based representations, time-series arrays, frequency-domain representations, wavelet decompositions, statistical distributions, probability density functions, feature maps, embedding spaces, dimensionality-reduced representations, manifolds, latent variable spaces, point clouds, voxel grids, octrees, mesh structures, or any combination thereof. The neural data structures may further represent different types of neural information, including spatial coordinates, temporal dynamics, frequency characteristics, phase relationships, correlation structures, connectivity patterns, network topologies, activation patterns, inhibition patterns, hemodynamic responses, metabolic activities, electrical potentials, receptor distributions, neurotransmitter concentrations, structural features, tissue properties, vascular arrangements, white matter tracts, or any other measurable or derivable neural properties. The neural data structures may be pre-processed, filtered, normalized, standardized, transformed, compressed, encrypted, error-corrected, noise-reduced, motion-compensated, artifact-removed, temporally or spatially aligned, interpolated, extrapolated, segmented, clustered, classified, or otherwise algorithmically manipulated to enhance their utility for subsequent analysis, visualization, or modulation purposes. Implementation of these data structures may use any suitable computational representation including fixed-point or floating-point numeric formats, binary encodings, quantized representations, or specialized data types optimized for neural data processing or machine learning operations. The one or more neural data structures can encompass spatial or temporal attributes, in addition to channel data, such as optical channel data. The one or more neural data structures can be dense neural data structures, or sparse neural data structures. The sparse neural data structures can store only voxels containing relevant neural information using coordinate-value pairs, and can be understood to be a sparse matrix or a sparse tensor. Sparse neural data structures can include sparse coordinate formats, indexed voxel collections, or point cloud representations. The one or more neural data structures can include radiofrequency (RF) data, in-phase quadrature (IQ) data, processed RF data, or processed IQ data. The one or more neural data structures can comprise data structures derived from ultrasound waves reflected, e.g., scattered, from the nervous system of the subject and received by the ultrasound transducer.

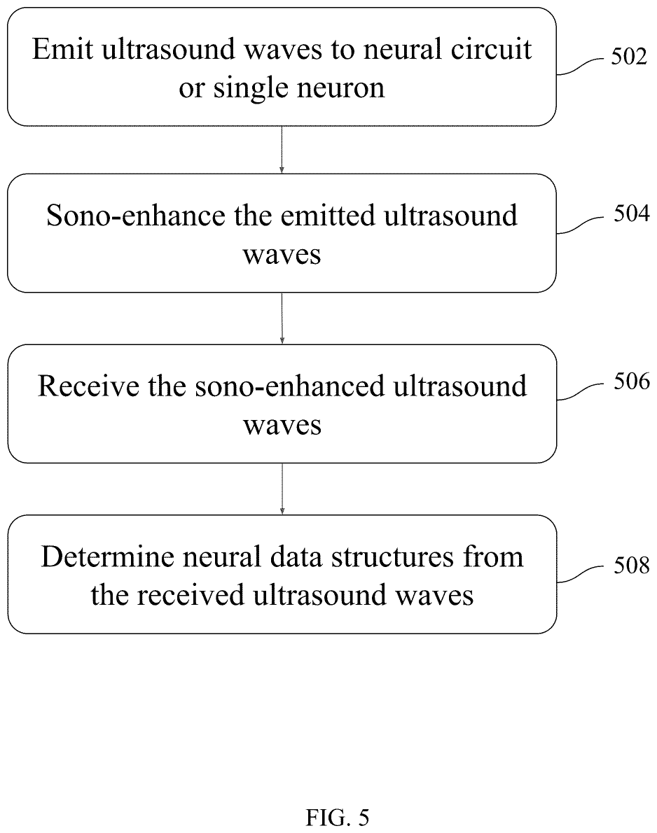

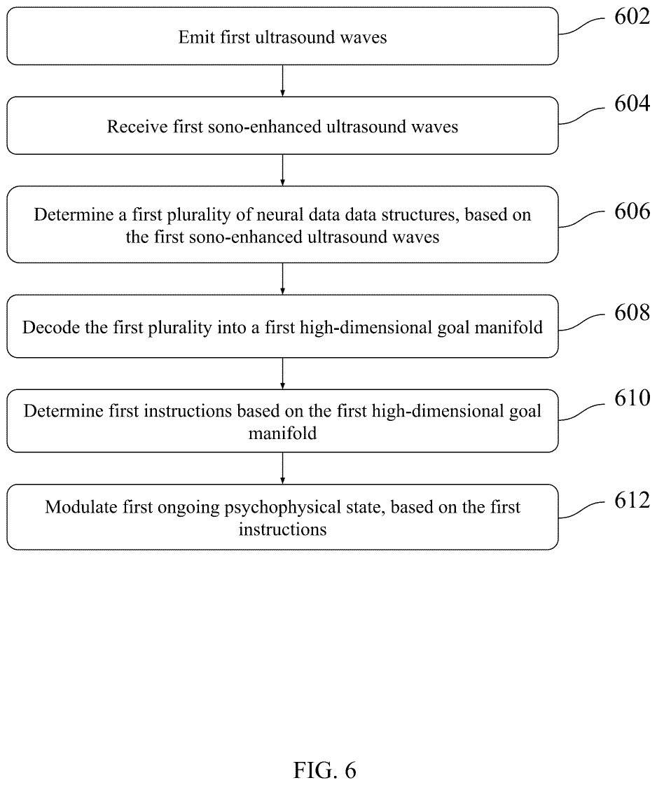

At 502 in FIG. 5, ultrasound waves are emitted to a neural circuit or single neuron of a subject, from an ultrasound transducer corresponding to the ultrasound transducer of any of the embodiments herein.

At 504 in FIG. 5, the emitted ultrasound waves are sono-enhanced, via the sono-enhancing contrast agent in the subject, to form sono-enhanced ultrasound waves. The sono-enhancing contrast agent can be genetically encoded, and can leverage the unique sonic properties of gas vesicles. The gas vesicles can comprise a protein shell. The gas vesicles can have an approximate width of 85 nm. The gas vesicles can contain a gas within the protein shell of the gas vesicle. The gas vesicles can comprise repeating molecular units, and each molecular unit can comprise two protein types, GvpA and GvpC. When acoustic waves of a specific frequency range, e.g., ultrasound waves, are applied to the gas vesicle, the gas vesicle can alter its conformation, e.g., buckle. Under the buckled conformation, the gas vesicle can provide non-linear (e.g., supralinear) amplified ultrasound wave signals, relative to the ultrasound waves that may enter and activate the ultrasound waves. Gas vesicles can possess GvpC proteins. GvpC-posessessing gas vesicles can be stiff, and may not easily alter their conformation, even when stimulated with acoustic waves of a given frequency range. In contrast, some gas vesicles, e.g., engineered gas vesicles, can lack GvpC proteins, e.g., via mutation or genetic engineering. GvpC-lacking gas vesicles can easily adjust their conformations, e.g., buckle, when stimulated with acoustic waves of a given frequency range, e.g., ultrasound waves. That is, GvpC-lacking gas vesicles can be more flexible in the presence of ultrasound waves, than a GvpC-positive gas vesicle, and can thus more readily enter a buckled conformation. The buckled conformation of a gas vesicle—GvpC-positive or GvpC-negative—when stimulated with ultrasound waves, can result in strongly reflected ultrasound waves of a particular frequency range, e.g., sono-enhanced ultrasound waves. These sono-enhanced ultrasound waves can provide a non-linear signal that can be readily detected by an ultrasound transducer, including the ultrasound transducer that emitted the original ultrasound waves. Alternatively or additionally, a different ultrasound transducer from the one that emitted the original ultrasound waves, can detect the sono-enhanced ultrasound waves. The use of sono-enhancing contrast agents, such as the gas vesicle-based contrast agents described in the present example, allow for the high signal-to-noise ratio leveraged by the scale-optimized ultrasound transducers described herein.

Sono-enhancing contrast agents can include ultrasonic reporters of calcium, voltage, neurotransmitters, or some other molecular indicator that can directly or indirectly proxy neural activity. An ultrasonic reporter of calcium, voltage, or neurotransmitters can provide sono-enhanced ultrasound waves, when both ultrasound waves and calcium are presented to the ultrasonic reporter. The ultrasonic reporter can be based on gas vesicles. The ultrasonic reporter can alter its conformation, e.g., the gas vesicle component of the ultrasonic reporter of calcium can buckle, in the presence of both the molecular indicator, e.g., calcium, a neurotransmitter, or a voltage change, and a specific range of ultrasound frequencies. The buckling can result in the sono-enhanced ultrasound waves being reflected back to the one or more ultrasound transducers, and the received sono-enhanced ultrasound waves can be processed as a detectable signal. The ultrasonic reporter can reflect the sono-enhanced ultrasound waves only when both the molecular indicator (e.g., calcium, neurotransmitter, or voltage change) and a specific frequency range of acoustic waves are presented to the ultrasonic reporter.

In the case of ultrasonic reporters of calcium, the calcium-gated mechanism of such reporters can be relevant to monitoring and modulating neural activity, because neural activity is associated with intracellular influxes of calcium. Therefore, the presence of ultrasonic reporters of calcium in the nervous system of a subject can result in the neural activity-specific signal of a specific ultrasound frequency range, and the neural activity-specific signal can be readily detected by the one or more ultrasound transducers. An ultrasonic reporter of calcium can comprise a calmodulin-binding protein (CBP), a calmodulin, and a gas vesicle comprising GvpC proteins. When an insufficient amount of calcium ions are proximate to the ultrasonic reporter of calcium, the gas vesicle is unlikely to buckle, even if ultrasound waves of a specific frequency range are presented to the ultrasonic reporter of calcium. In contrast, when both a sufficient amount of calcium ions and ultrasound waves of a specific frequency range are presented to the ultrasonic reporter of calcium, the gas vesicle can buckle. The buckling of the gas vesicle can result in the output of sono-enhanced ultrasound waves that can be readily detected as signal by one or more ultrasound transducers. The calcium-gated and stimulus ultrasound-gated buckling of the gas vesicle can be the result of conformational changes in the ultrasonic reporter of calcium, at the molecular level. That is, in the presence of both calcium and input ultrasound waves, the calmodulin and the calmodulin-binding protein of the ultrasonic reporter of calcium bind together, which can alter the conformation of the gas vesicle, which can cause the gas vesicle to buckle. The buckled gas vesicle can allow for the reflecting of sono-enhanced ultrasound waves back to the one or more ultrasound transducers.

At 506 in FIG. 5, the sono-enhanced ultrasound waves from the subject are received, by the ultrasound transducer.

At 508 in FIG. 5, one or more neural data structures are determined, using one or more processors, based on the received sono-enhanced ultrasound waves.

### Systems For Monitoring or Modulating One or More Portions of a Nervous System With a Sono-Enhancing Contrast Agent

Disclosed herein is a system for monitoring or modulating one or more portions of a nervous system from a subject, comprising: one or more ultrasound transducers, an ultrasound transducer of the one or more ultrasound transducers corresponding to the ultrasound transducer of any of the embodiments herein; a controller coupled to each of the one or more ultrasound transducers, the controller comprising a power source and a processor, wherein the power source is configured to power each of the one or more ultrasound transducers; and wherein the processor is configured to execute a method, the method comprising: sending one or more signals to the one or more ultrasound transducers; and receiving one or more neural data structures from the one or more ultrasound transducers.

### Methods of Modulating an Ongoing Psychophysical State Based on Ultrasound Monitoring of a Nervous System With a Sono-Enhancing Contrast Agent

Disclosed herein is a method for modulating a first ongoing psychophysical state of a subject, based on neural activity from one or more portions of a nervous system of a subject comprising a sono-enhancing contrast agent, comprising: emitting first ultrasound waves to the subject, from an ultrasound transducer corresponding to the ultrasound transducer of any of the embodiments herein; receiving first sono-enhanced ultrasound waves from the subject, wherein the first sono-enhanced ultrasound waves comprise the first emitted ultrasound waves enhanced via the sono-enhancing contrast agent; determining a first plurality of one or more neural data structures, using one or more processors, based on the first received sono-enhanced ultrasound waves; decoding the first plurality into a first high-dimensional goal manifold, via a predictive machine learning algorithm; determining first instructions based on the first high-dimensional goal manifold, via an instruction-determining machine learning algorithm; and modulating the first ongoing psychophysical state of the subject, based on the first instructions. The methods described herein can further comprise: emitting second ultrasound waves to the subject, from the ultrasound transducer; receiving second sono-enhanced ultrasound waves from the subject, wherein the second sono-enhanced ultrasound waves comprise the second emitted ultrasound waves enhanced via the sono-enhancing contrast agent; determining a second plurality of one or more neural data structures, using one or more processors, based on the second received sono-enhanced ultrasound waves; decoding the second plurality or the first plurality into a second high-dimensional goal manifold, via a predictive machine learning algorithm; determining second instructions based on the first high-dimensional goal manifold, via an instruction-determining machine learning algorithm; and modulating the second ongoing psychophysical state of the subject, based on the second instructions.

The method of modulating the ongoing psychophysical state of the subject based on ultrasound monitoring of a nervous system with a sono-enhancing contrast agent can utilize an advanced ultrasound-based neural interface to read neural data structures, e.g., neural data tensors, to perform sophisticated predictive modeling of brain states and future states of the world. The hardware implementation can leverage an ultrasonic transducer array, featuring elements ranging from 20 to 500 microns in width and operating at frequencies of 1-100 MHz. The ultrasound transducer can continuously emit and receive ultrasound waves enhanced by sono-enhancing contrast agents, generating high-resolution neural data tensors. Using advanced next-token prediction techniques inspired by language modeling approaches (such as transformers), the system can predict future neural states and inferred future states of the world by analyzing both current and historical neural data, augmented by available contextual metadata. Contextual metadata may include environmental data, prior user interactions, historical behaviors, and user-specific profiles. The method can perform predictive modeling, which can include the output of high-dimensional goal manifolds, which can be understood as structured representations encoding intricate intentions, conditional scenarios, preferences, and time-sensitive actions. This information can then be fed into an advanced artificial intelligence system capable of autonomously or semi-autonomously executing tasks. By aligning with the decoded intention manifolds, the artificial intelligence (AI) can ensure that executed tasks precisely match the user's conceptual expectations, constraints, and individualized values. In addition, the ultrasound hardware used for the method's neuromodulation capabilities can deliver intuitive physiological updates directly to the subject. The modulation loop can convey real-time status, alerts, or requests for further input, allowing users to dynamically refine their neural intent manifolds. Such bi-directional interaction creates a seamless, adaptive, and deeply personalized human-machine interface, enabling fluid collaboration between users and intelligent systems.

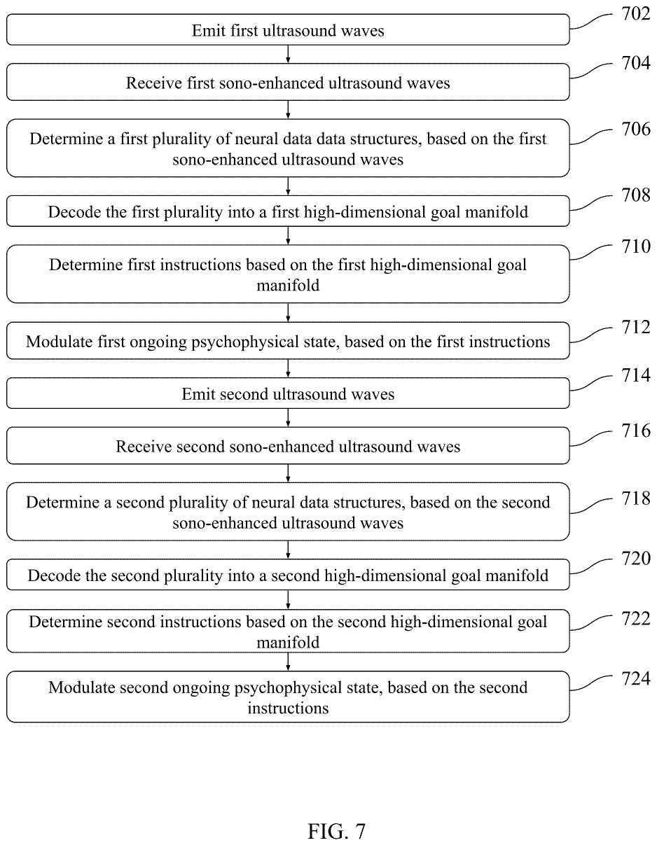

At 602 in FIG. 6 or 702 in FIG. 7, first ultrasound waves are emitted to the subject, from an ultrasound transducer corresponding to the ultrasound transducer of any of the embodiments herein. The element in the ultrasonic transducer array can be circular, elliptical, square, rectangular, hexagonal, regular, or irregular, in shape. The width of an element in the ultrasonic transducer array can refer to a diameter, a cross-length, a length, or some other dimension relevant to the geometry of the ultrasonic transducer array element.

The ultrasonic transducer array can be a capacitive micromachined ultrasonic transducer (CMUT) array or a piezoelectric micromachined ultrasonic transducer (PMUT) array. The CMUT array can comprise a flat membrane CMUT, a curved membrane CMUT, a dual-electrode CMUT, a collapsed-mode CMUT, a wafer-bonded CMUT, a surface-micromachined CMUT, a CMUT-on-CMOS integrated device, a piston-shaped CMUT, an annular array CMUT, or a flexible CMUT. In some embodiments, the PMUT array can comprise a unimorph PMUT, a bimorph PMUT, a dome-shaped PMUT, a suspended membrane PMUT, an aluminum nitride PMUT, a lead zirconate titanate PMUT, a scandium-doped aluminum nitride PMUT, a lithium niobate PMUT, a polyvinylidene fluoride PMUT, a thin-film PMUT, or a flexible PMUT.

At 604 in FIG. 6 or 704 in FIG. 7, first sono-enhanced ultrasound waves are received from the subject, wherein the first sono-enhanced ultrasound waves comprise the first emitted ultrasound waves enhanced via the sono-enhancing contrast agent. The sono-enhancing contrast agent can comprise a biological sono-enhancing contrast agent, a polymeric sono-enhancing contrast agent, or a pharmaceutical sono-enhancing contrast agent. The biological sono-enhancing contrast agent can comprise a genetically-encoded sono-enhancing contrast agent. The genetically-encoded sono-enhancing contrast agent can comprise an ultrasonic reporter of calcium, an ultrasonic reporter of voltage, or an ultrasonic reporter of a neurotransmitter, e.g., glutamate, GABA, dopamine, or acetylcholine. The ultrasonic reporter of calcium can be based on calcium-gated binding proteins, such as calmodulin and calmodulin-binding proteins. The sono-enhancing contrast agent can also leverage the physical properties of gas vesicles. For example, the altering of gas vesicles' conformations, e.g., “buckling”, upon stimulation by ultrasound waves of a specific frequency range, can reflect back sono-enhanced ultrasound waves towards one or more ultrasound transducers. Accordingly, the sono-enhancing contrast agent can comprise gas vesicles. The gas vesicles of the sono-enhancing contrast agent can be modified to buckle at different susceptibilities, e.g., at different strengths (e.g., amplitudes) of ultrasound waves. For example, the gas vesicle's protein shell may or may not comprise GvpC or GvpA proteins. In addition, the sono-enhancing contrast agent can be expressed in accordance with different temporal and/or spatial patterns in the subject. For example, for temporal gating, the sono-enhancing contrast agent can be activated, e.g., via an inducible promoter. For spatial gating, the sono-enhancing contrast agent can be localized, e.g., via a tissue-specific promoter. Both temporal and spatial gating can be used to impose further conditions that control the activation, and thus utility, of the sono-enhancing contrast agent. Accordingly, the condition on calcium for the activation of ultrasonic reporters of calcium can be understood to be a temporal-spatial gating on the sono-enhancing contrast agent. The sono-enhancing contrast agent can comprise a polymeric sono-enhancing contrast agent. The polymeric sono-enhancing contrast agent can comprise a silica-based particle. The silica-based particle can include a solid silica core, an intermediate film of polymethacrylic acid (PMA) that can be cross-linked or pH-responsive, and an outer porous organosilica shell. The polymeric sono-enhancing contrast agent can indicate the neural activity of a subject by exploiting the intracellular or extracellular pH changes associated with neural activity in a subject. For example, the carboxyl group of the polymeric contrast agent, e.g., the PMA-based silica particle, may protonate under acidic pH, which can cause the film of PMA to collapse, and thereby alter the acoustic properties of the polymeric sono-enhancing contrast agent. In contrast, at neutral pH, the carboxyl groups of the PMA-based silica particle may deprotonated, which can cause the carboxyl groups to repel each other, and thereby cause the film of PMA to swell with water. The polymeric sono-enhancing contrast agent can be further engineered to respond directly to physiological indicators of neural activity, such as cellular changes in voltage or calcium in neurons. The polymeric sono-enhancing contrast agent can be delivered into the nervous system, e.g., the brain, of the subject via crossing the blood-brain barrier of the subject.

The sono-enhancing contrast agent may be expressed in a pattern that is broader than the neural scale for which the scale-optimized ultrasound transducer is optimized. That is, for an ultrasound transducer optimized for brain region-specific imaging, the pattern of the sono-enhancing contrast agent may be ubiquitous across the brain, but such a broad expression pattern would not necessarily impede the target imaging or modulating scale of the ultrasound transducer, because some of the sono-enhancing contrast agent-expressing cells may be outside the field of view of the one or more ultrasound transducers, and/or because the sono-enhancing contrast agent may not be activated due to spatial or temporal gatings.

At 606 in FIG. 6 or 706 in FIG. 7, a first plurality of one or more neural data structures, using one or more processors, based on the first received sono-enhanced ultrasound waves. The one or more neural data structures can be any data structure that can be represented as one or more arrays or collections of data points. For example, the one or more neural data structures can comprise tensors, where the tensors can relate to ultrasound patterns corresponding to the nervous system of the subject. These data structures may include vectors (one-dimensional arrays), traditional matrices (two-dimensional arrays), three-dimensional arrays, multi-dimensional tensors, sparse matrices, compressed representations, hierarchical data structures, graph-based representations, time-series arrays, frequency-domain representations, wavelet decompositions, statistical distributions, probability density functions, feature maps, embedding spaces, dimensionality-reduced representations, manifolds, latent variable spaces, point clouds, voxel grids, octrees, mesh structures, or any combination thereof. The neural data structures may further represent different types of neural information, including spatial coordinates, temporal dynamics, frequency characteristics, phase relationships, correlation structures, connectivity patterns, network topologies, activation patterns, inhibition patterns, hemodynamic responses, metabolic activities, electrical potentials, receptor distributions, neurotransmitter concentrations, structural features, tissue properties, vascular arrangements, white matter tracts, or any other measurable or derivable neural properties. The neural data structures may be pre-processed, filtered, normalized, standardized, transformed, compressed, encrypted, error-corrected, noise-reduced, motion-compensated, artifact-removed, temporally or spatially aligned, interpolated, extrapolated, segmented, clustered, classified, or otherwise algorithmically manipulated to enhance their utility for subsequent analysis, visualization, or modulation purposes. Implementation of these data structures may use any suitable computational representation including fixed-point or floating-point numeric formats, binary encodings, quantized representations, or specialized data types optimized for neural data processing or machine learning operations. The one or more neural data structures can encompass spatial or temporal attributes, in addition to channel data, such as optical channel data. The one or more neural data structures can be dense neural data structures, or sparse neural data structures. The sparse neural data structures can store only voxels containing relevant neural information using coordinate-value pairs, and can be understood to be a sparse matrix or a sparse tensor. Sparse neural data structures can include sparse coordinate formats, indexed voxel collections, or point cloud representations. The one or more neural data structures can include radiofrequency (RF) data, in-phase quadrature (IQ) data, processed RF data, or processed IQ data. The one or more neural data structures can comprise data structures derived from ultrasound waves reflected, e.g., scattered, from the nervous system of the subject and received by the ultrasound transducer.

At 608 in FIG. 6 or 708 in FIG. 7, the first plurality is decoded into a first high-dimensional goal manifold, via an instruction-determining machine learning algorithm. The first high-dimensional goal manifold can comprise dimensions representing, at least in part, predictions across multiple timescales, environmental variables, or personal values of the subject. The multiple timescales can comprise milliseconds-long timescales, seconds-long timescales, minutes-long timescales, hours-long timescales. days-long timescales, weeks-long timescales, months-long timescales, or years-long timescales. High-dimensional goal manifolds, e.g., the first high-dimensional goal manifold can be understood as the result of decoding neural activity into complex and comprising representations that can include rich descriptions of what an individual desires or intends. The high-dimensional goal manifolds can capture not just immediate needs of the subject, but also highly structured intentions of the subject, which can span the multiple timescales described above. Although the high-dimensional goal manifolds can indeed have high dimensionality, it is worth noting that the number of dimensions comprised in the high-dimensional goal manifolds can—and often will—possess fewer dimensions than the number of dimensions comprised by the original neural activity of the subject, from which the high-dimensional goal manifolds derive. The original neural activity of the subject can be understood to have n dimensions, where n can correspond to the number of single units of neural activity. That is, the high dimensional aspect of the high-dimensional goal manifolds can be understood to be relative. The high-dimensional goal manifolds can be understood to be relatively high dimensional when compared to the dimensionality of representations resulting from conventional neural decoding approaches, which often capture simplistic concepts. For example, a conventional neural decoding approach may result in a simple representation for a subject, such as “the subject wants coffee”. In contrast, the high-dimensional goal manifolds described herein can comprise richer descriptions, such as the multiple timescales, environmental variables, or personal values of the subject. The environmental variables, e.g., explicit external world states, can comprise the clear specifying of desired outcomes in the external environment. The personal values of the can also be aligned, such that the decoding algorithms used to generate the high-dimensional goal manifolds can be tailored to the subject's individual ethics, preferences, or personal values. For example, rather than encoding a representation limited to the subject wanting a coffee, the high-dimensional goal manifolds described herein may represent a description at least as complex as “the subject wants a hot vanilla latte from Borks Coffee, with half the sweetener and no sleeve. The subject would like to tip 15% of the coffee price, but only if doing so does not cause the subject to run late. Also, the subject should be warmed up five minutes before the subject leaves, and the subject's departure should be precisely timed such that the subject arrives just as the order is ready. If the subject is going to be late, the subject should be reminded to grab an ice coffee from the fridge, before they leave.” Such a representation is far more high dimensional than the dimensionality of conventional representations deriving from neural activity, such as “the subject wants coffee”. The high-dimensional goal manifolds can contribute to a method that forms an advanced closed-loop neural decoding and modulating system that is engineered to allow for seamless and deeply intuitive communication between an individual and artificial intelligence (AI) systems. The generating and continuous updating of the high-dimensional goal manifolds empowers AI systems to autonomously or semi-autonomously execute complex context-aware tasks in a manner that can remain deeply aligned with the individual's personal intentions, values, and preferences. The method described herein can comprise a feedback-driven learning loop, continuous refinement of predictive models based on observed neural, physiological, or behavioral outcomes. The method described herein can also comprise bidirectional AI interactions, such that the modulation capabilities for intuitive communication exist between the AI systems and the subject, thereby allowing for real-time updates and feedback from the subject. The methods described herein can also include safety, ethical, and security mechanisms. Built-in protections that ensure user safety, ethical operations, and security against unintended or unauthorized uses can be included.

At 610 in FIG. 6 or 710 in FIG. 7, first instructions based on the first high-dimensional goal manifold is determined, via an instruction-determining machine learning algorithm.

At 612 in FIG. 6 or 712 in FIG. 7, the first ongoing psychophysical state of the subject is modulated, based on the first instructions. The modulating of one or more portions of the nervous system of the subject by the methods described herein, e.g., via an ultrasound transducer, can provide an intuitive communication channel back to the user. The modulating can subtly convey updates, prompt attention, or provide new information directly through changes in the subject's neural state, which can allow for real-time adjustment or refinement of goals or tasks. Such a two-way interaction can allow the user and AI systems to remain synchronized in their understanding, which can create a uniquely intuitive human-machine interface that can continuously adapt to evolving circumstances. The method described herein can blend neural decoding, sophisticated AI autonomy, and intuitive neuromodulation into a cohesive, deeply personalized interface capable of profoundly enhancing human productivity, autonomy, and quality of life.

Disclosed herein is a method for modulating a second ongoing psychophysical state of the subject, further comprising: emitting second ultrasound waves to the subject, from the ultrasound transducer; receiving second sono-enhanced ultrasound waves from the subject, wherein the second sono-enhanced ultrasound waves comprise the second emitted ultrasound waves enhanced via the sono-enhancing contrast agent; determining a second plurality of one or more neural data structures, using one or more processors, based on the second received sono-enhanced ultrasound waves; decoding the second plurality or the first plurality into a second high-dimensional goal manifold, via a predictive machine learning algorithm; determining second instructions based on the first high-dimensional goal manifold, via an instruction-determining machine learning algorithm; and modulating the second ongoing psychophysical state of the subject, based on the second instructions.

At 714 in FIG. 7, second ultrasound waves are emitted to the one or more portions of the nervous system, from the ultrasound transducer.

At 716 in FIG. 7, second sono-enhanced ultrasound waves from the subject are received, wherein the second sono-enhanced ultrasound waves comprise the second emitted ultrasound waves enhanced via the sono-enhancing contrast agent.

At 718 in FIG. 7, a second plurality of one or more neural data structures is determined, using the one or more processors, based on the second received sono-enhanced ultrasound waves.

At 720 in FIG. 7, the second plurality or the first plurality is decoded into a second high-dimensional goal manifold, via a predictive machine learning algorithm. The second high-dimensional goal manifold can comprise dimensions representing, at least in part, predictions across multiple timescales, environmental variables, or personal values of the subject. The multiple timescales can comprise milliseconds-long timescales, seconds-long timescales, minutes-long timescales, hours-long timescales. days-long timescales, weeks-long timescales, months-long timescales, or years-long timescales. High-dimensional goal manifolds, e.g., the second high-dimensional goal manifold can be understood as the result of decoding neural activity into complex and comprising representations that can include rich descriptions of what an individual desires or intends. The high-dimensional goal manifolds can capture not just immediate needs of the subject, but also highly structured intentions of the subject, which can span the multiple timescales described above. Although the high-dimensional goal manifolds can indeed have high dimensionality, it is worth noting that the number of dimensions comprised in the high-dimensional goal manifolds can—and often will—possess fewer dimensions than the number of dimensions comprised by the original neural activity of the subject, from which the high-dimensional goal manifolds derive. The original neural activity of the subject can be understood to have n dimensions, where n can correspond to the number of single units of neural activity. That is, the high dimensional aspect of the high-dimensional goal manifolds can be understood to be relative. The high-dimensional goal manifolds can be understood to be relatively high dimensional when compared to the dimensionality of representations resulting from conventional neural decoding approaches, which often capture simplistic concepts. For example, a conventional neural decoding approach may result in a simple representation for a subject, such as “the subject wants coffee”. In contrast, the high-dimensional goal manifolds described herein can comprise richer descriptions, such as the multiple timescales, environmental variables, or personal values of the subject. The environmental variables, e.g., explicit external world states, can comprise the clear specifying of desired outcomes in the external environment. The personal values of the can also be aligned, such that the decoding algorithms used to generate the high-dimensional goal manifolds can be tailored to the subject's individual ethics, preferences, or personal values. For example, rather than encoding a representation limited to the subject wanting a coffee, the high-dimensional goal manifolds described herein may represent a description at least as complex as “the subject wants a hot vanilla latte from Borks Coffee, with half the sweetener and no sleeve. The subject would like to tip 15% of the coffee price, but only if doing so does not cause the subject to run late. Also, the subject should be warmed up five minutes before the subject leaves, and the subject's departure should be precisely timed such that the subject arrives just as the order is ready. If the subject is going to be late, the subject should be reminded to grab an ice coffee from the fridge, before they leave.” Such a representation is far more high dimensional than the dimensionality of conventional representations deriving from neural activity, such as “the subject wants coffee”. The high-dimensional goal manifolds can contribute to a method that forms an advanced closed-loop neural decoding and modulating system that is engineered to allow for seamless and deeply intuitive communication between an individual and artificial intelligence (AI) systems. The generating and continuous updating of the high-dimensional goal manifolds empowers AI systems to autonomously or semi-autonomously execute complex context-aware tasks in a manner that can remain deeply aligned with the individual's personal intentions, values, and preferences. The method described herein can comprise a feedback-driven learning loop, continuous refinement of predictive models based on observed neural, physiological, or behavioral outcomes. The method described herein can also comprise bidirectional AI interactions, such that the modulation capabilities for intuitive communication exist between the AI systems and the subject, thereby allowing for real-time updates and feedback from the subject. The methods described herein can also include safety, ethical, and security mechanisms. Built-in protections that ensure user safety, ethical operations, and security against unintended or unauthorized uses can be included.

At 722 in FIG. 7, second instructions based on the second high-dimensional goal manifold or the first high-dimensional goal manifold are determined, via an instruction-determining machine learning algorithm. The second high-dimensional goal manifold can comprise dimensions representing, at least in part, predictions across multiple timescales, environmental variables, or personal values of the subject. The multiple timescales can comprise milliseconds-long timescales, seconds-long timescales, minutes-long timescales, hours-long timescales. days-long timescales, weeks-long timescales, months-long timescales, or years-long timescales

At 724 in FIG. 7, the second ongoing psychophysical state of the subject is modulated, based on the second instructions.

Disclosed herein is a method for modulating a first ongoing physiological state of a subject, based on neural activity from one or more portions of a nervous system of a subject comprising a sono-enhancing contrast agent, comprising: emitting first ultrasound waves to the subject, from an ultrasound transducer corresponding to the ultrasound transducer of any of the embodiments herein, to the one or more portions of the nervous system; receiving first sono-enhanced ultrasound waves from the subject, wherein the first sono-enhanced ultrasound waves comprise the first emitted ultrasound waves enhanced via the sono-enhancing contrast agent; determining a first plurality of one or more neural data structures, using one or more processors, based on the first received sono-enhanced ultrasound waves; decoding the first plurality, via an instruction-determining machine learning algorithm, into first instructions; and modulating the first ongoing psychophysical state of the subject, based on the first instructions. The method can further comprise: emitting second ultrasound waves to the subject, from an ultrasound transducer corresponding to the ultrasound transducer of any of the embodiments herein, to the one or more portions of the nervous system; receiving second sono-enhanced ultrasound waves from the subject, wherein the second sono-enhanced ultrasound waves comprise the second emitted ultrasound waves enhanced via the sono-enhancing contrast agent; determining a second plurality of one or more neural data structures, using one or more processors, based on the second received sono-enhanced ultrasound waves; decoding the second plurality, via an instruction-determining machine learning algorithm, into second instructions; and modulating the second ongoing psychophysical state of the subject, based on the second instructions.

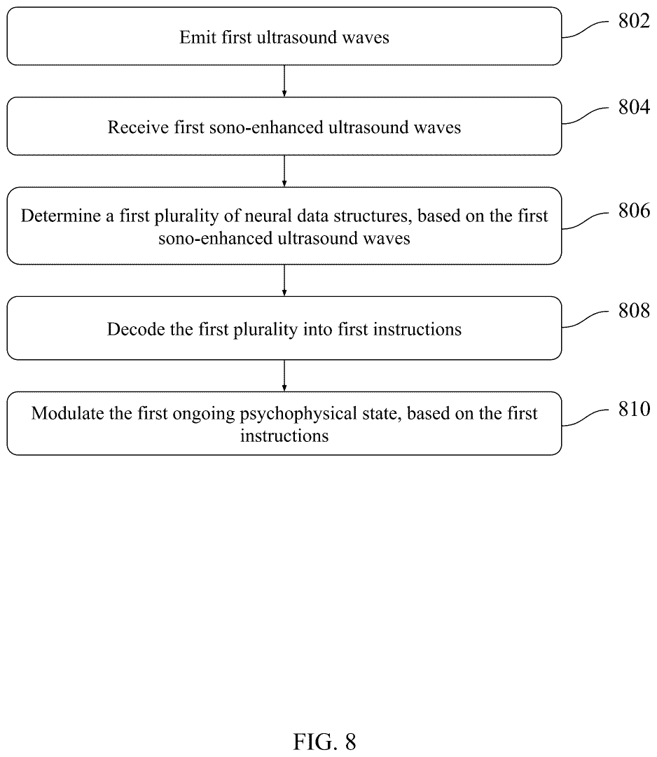

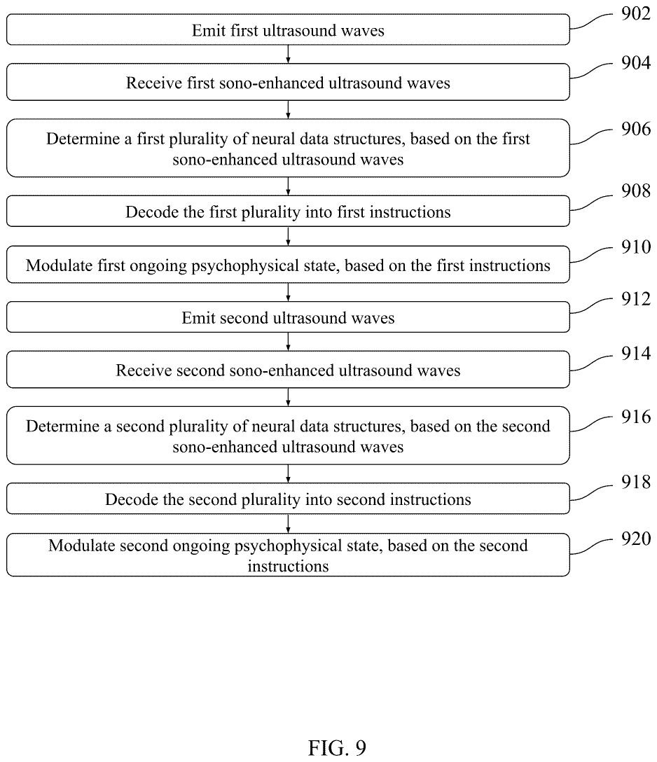

At 802 in FIG. 8 or 902 in FIG. 9, first ultrasound waves are emitted to the subject, from an ultrasound transducer corresponding to the ultrasound transducer of any of embodiments herein, to the one or more portions of the nervous system. The element in the ultrasonic transducer array can be circular, elliptical, square, rectangular, hexagonal, regular, or irregular, in shape. The width of an element in the ultrasonic transducer array can refer to a diameter, a cross-length, a length, or some other dimension relevant to the geometry of the ultrasonic transducer array element.

The ultrasonic transducer array can be a capacitive micromachined ultrasonic transducer (CMUT) array or a piezoelectric micromachined ultrasonic transducer (PMUT) array. The CMUT array can comprise a flat membrane CMUT, a curved membrane CMUT, a dual-electrode CMUT, a collapsed-mode CMUT, a wafer-bonded CMUT, a surface-micromachined CMUT, a CMUT-on-CMOS integrated device, a piston-shaped CMUT, an annular array CMUT, or a flexible CMUT. In some embodiments, the PMUT array can comprise a unimorph PMUT, a bimorph PMUT, a dome-shaped PMUT, a suspended membrane PMUT, an aluminum nitride PMUT, a lead zirconate titanate PMUT, a scandium-doped aluminum nitride PMUT, a lithium niobate PMUT, a polyvinylidene fluoride PMUT, a thin-film PMUT, or a flexible PMUT.

The control circuit configured to receive or execute instructions for activating the modulating mode or the monitoring mode can refer to any hardware, software, firmware, or combination thereof capable of managing, directing, or regulating the operation of the ultrasound transducer. The control circuit may include one or more microprocessors, microcontrollers, field-programmable gate arrays (FPGAs), application-specific integrated circuits (ASICs), digital signal processors (DSPs), programmable logic devices (PLDs), complex programmable logic devices (CPLDs), system-on-chip (SoC) devices, embedded controllers, state machines, sequencers, logic circuits, analog circuits, digital circuits, mixed-signal circuits, timing circuits, counters, registers, memory devices, buses, input/output (I/O) interfaces, signal conditioning circuitry, amplifiers, filters, oscillators, phase-locked loops (PLLs), clock generators, power management circuits, wireless communication modules, wired communication interfaces, multiplexers, demultiplexers, analog-to-digital converters (ADCs), digital-to-analog converters (DACs), voltage regulators, current sources, switching elements, or any combination thereof. The control circuit may be centralized in a single location or distributed across multiple components or subsystems. The control circuit may be implemented on one or more printed circuit boards (PCBs), integrated circuits, multi-chip modules, flexible circuits, rigid-flex circuits, ceramic substrates, or other suitable substrates. The control circuit may execute firmware, operating systems, real-time operating systems, device drivers, middleware, application software, neural network models, machine learning algorithms, or signal processing routines. The control circuit may be configured to receive instructions from external sources including, but not limited to, wired connections, wireless connections, optical connections, radiofrequency identification (RFID), near-field communication (NFC), Bluetooth, Bluetooth Low Energy, WiFi, WiFi HaLow, cellular networks, satellite communications, infrared, ultrasonic communication, optical data communication, Light Fidelity (Li-Fi), ultra-wideband, impulse-radio ultra-wideband, ultrasonic data transmission, wireless chip-to-chip, manual inputs, millimeter wave technologies, or a combination thereof. The control circuit may further include error detection and correction mechanisms, self-diagnostic capabilities, calibration functionalities, fault tolerance features, redundancy systems, and security measures to protect against unauthorized access or interference. The control circuit may operate in open-loop or closed-loop configurations, with or without feedback mechanisms for maintaining desired operational parameters.

The modulating mode that may be activated by an embodiment of the ultrasound transducer can refer to any operational state, setting, configuration, or functionality that enables the ultrasound transducer to influence, alter, adjust, stimulate, regulate, modify, control, manipulate, perturb, or otherwise affect the activity, function, state, or properties of one or more portions of the nervous system. The modulating mode via the ultrasound transducers may encompass, thermal modulation, mechanical modulation, acoustic modulation, pressure modulation, neuromodulation, sonication, focused ultrasound stimulation, pulsed ultrasound delivery, continuous wave ultrasound delivery, structured ultrasound pattern delivery, spatiotemporal ultrasound pattern delivery, frequency-specific ultrasound delivery, amplitude-modulated ultrasound delivery, phase-modulated ultrasound delivery, beam-steered ultrasound delivery, or any combination thereof. The modulating mode may operate at multiple spatial scales ranging from whole-brain modulation to region-specific modulation to circuit-specific modulation to single-neuron modulation. The effects produced by the modulating mode may include, but are not limited to, excitation, inhibition, facilitation, suppression, potentiation, depression, synchronization, desynchronization, entrainment, phase-resetting, frequency-shifting, resonance, plasticity induction, blood-brain barrier opening, drug delivery enhancement, gene expression alteration, protein conformation changes, ion channel activation or deactivation, neurotransmitter release modulation, neurovasculature response modulation, glial cell activation, neurogenesis modulation, or any neurophysiological response. The modulating mode may be implemented using various ultrasound parameters and delivery patterns including specific frequencies, amplitudes, pulse repetition frequencies, duty cycles, waveforms, pulse shapes, pulse durations, inter-pulse intervals, acoustic pressure levels, focal geometries, beam profiles, intensity distributions, energy densities, treatment durations, sonication sequences, or combinatorial protocols. The modulating mode may operate independently or in coordination with other therapeutic modalities, diagnostic procedures, or feedback mechanisms. The modulating mode may further incorporate adaptive algorithms, closed-loop control, subject-specific customization, treatment planning optimization, real-time adjustment capabilities, safety monitoring, or dosimetry management.

The monitoring mode that may be activated by an embodiment of the ultrasound transducer can refer to any operational state, setting, configuration, or functionality that enables the ultrasound transducer to detect, sense, measure, observe, record, analyze, assess, characterize, interpret, track, or otherwise obtain information about the activity, function, state, structure, composition, dynamics, or properties of one or more portions of the nervous system. The monitoring mode may encompass anatomical imaging, functional imaging, structural imaging, hemodynamic imaging, perfusion imaging, flow imaging, elastography, spectroscopy, velocimetry, thermometry, neurosonography, acoustic angiography, tissue characterization, contrast-enhanced imaging, molecular imaging, receptor imaging, metabolic imaging, diffusion imaging, connectivity imaging, or any combination thereof. The monitoring mode may operate at multiple spatial scales ranging from whole-brain monitoring to region-specific monitoring to circuit-specific monitoring to single-neuron monitoring. The information acquired during monitoring mode may include, but is not limited to, neural activation patterns, neural inhibition patterns, neural oscillations, network synchronization, functional connectivity, structural connectivity, blood flow dynamics, vascular architecture, tissue microstructure, cell morphology, cell density, extracellular matrix properties, molecular signatures, protein expression, receptor distribution, neurotransmitter dynamics, metabolite concentrations, tissue oxygenation, temperature distributions, mechanical properties, electrical properties, developmental changes, pathological alterations, treatment responses, or any neurophysiological parameters. The monitoring mode may be implemented using various ultrasound techniques including B-mode imaging, M-mode imaging, Doppler imaging, color flow imaging, power Doppler imaging, harmonic imaging, contrast-enhanced imaging, pulse-echo imaging, through-transmission imaging, 3D/4D imaging, superresolution imaging, acoustic microscopy, photoacoustic imaging, magnetoacoustic imaging, elastographic imaging, or multimodal approaches. The monitoring mode may operate at various temporal resolutions including real-time, near-real-time, high frame rate, time-averaged, gated, or time-lapse monitoring. The monitoring mode may further incorporate signal processing techniques, image reconstruction algorithms, computational modeling, statistical analysis, machine learning approaches, artificial intelligence, feature extraction, pattern recognition, classification methods, segmentation algorithms, registration procedures, motion compensation, artifact reduction, noise suppression, or data fusion strategies to enhance the extraction, interpretation, or utilization of neural information.

The processing circuit configured to transform the received ultrasound waves can refer to any hardware, software, firmware, or combination thereof capable of performing operations, computations, transformations, analyses, or manipulations on data derived from the ultrasound waves. The processing circuit may include, one or more microprocessors, microcontrollers, central processing units (CPUs), graphics processing units (GPUs), tensor processing units (TPUs), digital signal processors (DSPs), field-programmable gate arrays (FPGAs), application-specific integrated circuits (ASICs), system-on-chip (SoC) devices, neuromorphic processors, quantum processors, cloud computing resources, distributed computing systems, parallel processing arrays, vector processors, arithmetic logic units (ALUs), co-processors, accelerators, memory controllers, cache systems, register files, instruction decoders, pipeline architectures, branch predictors, specialized computing elements, programmable logic devices (PLDs), complex programmable logic devices (CPLDs), analog circuits, digital circuits, mixed-signal circuits, or any combination thereof. The processing circuit may implement various signal processing operations including, but not limited to, filtering, amplification, denoising, beamforming, pulse compression, envelope detection, log compression, time gain compensation, dynamic range adjustment, scan conversion, image reconstruction, feature extraction, segmentation, registration, motion compensation, phase detection, frequency analysis, time-domain analysis, spatial-domain analysis, transform-domain processing, statistical analysis, parametric estimation, dimensionality reduction, or data fusion. The processing circuit may employ algorithms including adaptive filters, wavelet transforms, Fourier transforms, compressive sensing, sparse representations, machine learning, deep learning, neural networks, probabilistic models, statistical models, physical models, biomechanical models, inverse problem solving, regularization techniques, optimization methods, classification algorithms, clustering algorithms, pattern recognition, spectral analysis, temporal analysis, spatial analysis, or multiscale analysis. The processing circuit may be implemented on one or more printed circuit boards (PCBs), integrated circuits, multi-chip modules, flexible circuits, rigid-flex circuits, ceramic substrates, or other suitable substrates. The processing circuit may be centralized in a single location or distributed across multiple components or subsystems, and may include cloud-based or edge computing architectures. The processing circuit may further incorporate error handling, parallel processing, pipelined processing, real-time processing, batch processing, high-performance computing techniques, memory management, data storage, caching strategies, or energy optimization methods. The processing circuit may interact with other system components through various interfaces, buses, networks, or communication protocols to receive input data, provide output results, or exchange intermediate information.

At 804 in FIG. 8 or 904 in FIG. 9, first sono-enhanced ultrasound waves from the subject are received, wherein the first sono-enhanced ultrasound waves comprise the first emitted ultrasound waves enhanced via the sono-enhancing contrast agent. The sono-enhancing contrast agent can comprise a biological sono-enhancing contrast agent, a polymeric sono-enhancing contrast agent, or a pharmaceutical sono-enhancing contrast agent. The biological sono-enhancing contrast agent can comprise a genetically-encoded sono-enhancing contrast agent. The genetically-encoded sono-enhancing contrast agent can comprise an ultrasonic reporter of calcium, an ultrasonic reporter of voltage, or an ultrasonic reporter of a neurotransmitter, e.g., glutamate, GABA, dopamine, or acetylcholine. The ultrasonic reporter of calcium can be based on calcium-gated binding proteins, such as calmodulin and calmodulin-binding proteins. The sono-enhancing contrast agent can also leverage the physical properties of gas vesicles. For example, the altering of gas vesicles' conformations, e.g., “buckling”, upon stimulation by ultrasound waves of a specific frequency range, can reflect back sono-enhanced ultrasound waves towards one or more ultrasound transducers. Accordingly, the sono-enhancing contrast agent can comprise gas vesicles. The gas vesicles of the sono-enhancing contrast agent can be modified to buckle at different susceptibilities, e.g., at different strengths (e.g., amplitudes) of ultrasound waves. For example, the gas vesicle's protein shell may or may not comprise GvpC or GvpA proteins. In addition, the sono-enhancing contrast agent can be expressed in accordance with different temporal and/or spatial patterns in the subject. For example, for temporal gating, the sono-enhancing contrast agent can be activated, e.g., via an inducible promoter. For spatial gating, the sono-enhancing contrast agent can be localized, e.g., via a tissue-specific promoter. Both temporal and spatial gating can be used to impose further conditions that control the activation, and thus utility, of the sono-enhancing contrast agent. Accordingly, the condition on calcium for the activation of ultrasonic reporters of calcium can be understood to be a temporal-spatial gating on the sono-enhancing contrast agent. The sono-enhancing contrast agent can comprise a polymeric sono-enhancing contrast agent. The polymeric sono-enhancing contrast agent can comprise a silica-based particle. The silica-based particle can include a solid silica core, an intermediate film of polymethacrylic acid (PMA) that can be cross-linked or pH-responsive, and an outer porous organosilica shell. The polymeric sono-enhancing contrast agent can indicate the neural activity of a subject by exploiting the intracellular or extracellular pH changes associated with neural activity in a subject. For example, the carboxyl group of the polymeric contrast agent, e.g., the PMA-based silica particle, may protonate under acidic pH, which can cause the film of PMA to collapse, and thereby alter the acoustic properties of the polymeric sono-enhancing contrast agent. In contrast, at neutral pH, the carboxyl groups of the PMA-based silica particle may deprotonated, which can cause the carboxyl groups to repel each other, and thereby cause the film of PMA to swell with water. The polymeric sono-enhancing contrast agent can be further engineered to respond directly to physiological indicators of neural activity, such as cellular changes in voltage or calcium in neurons. The polymeric sono-enhancing contrast agent can be delivered into the nervous system, e.g., the brain, of the subject via crossing the blood-brain barrier of the subject. The pharmaceutical sono-enhancing contrast agent can include a small molecule, e.g., a drug. The pharmaceutical sono-enhancing contrast agent can be functionally coupled to a physiological indicator of neural activity, such as intracellular or extracellular calcium changes, intracellular or extracellular voltage changes, or intracellular or extracellular changes in a neurotransmitter. The pharmaceutical contrast agent can be delivered into the nervous system, e.g., the brain, of the subject via crossing the blood-brain barrier of the subject.

The sono-enhancing contrast agent may be expressed in a pattern that is broader than the neural scale for which the scale-optimized ultrasound transducer is optimized. That is, for an ultrasound transducer optimized for brain region-specific imaging, the pattern of the sono-enhancing contrast agent may be ubiquitous across the brain, but such a broad expression pattern would not necessarily impede the target imaging or modulating scale of the ultrasound transducer, because some of the sono-enhancing contrast agent-expressing cells may be outside the field of view of the one or more ultrasound transducers, and/or because the sono-enhancing contrast agent may not be activated due to spatial or temporal gatings.

At 806 in FIG. 8 or 906 in FIG. 9, a first plurality of one or more neural data structures is determined, using one or more processors, based on the first received sono-enhanced ultrasound waves. The one or more neural data structures can be any data structure that can be represented as one or more arrays or collections of data points. For example, the one or more neural data structures can comprise tensors, where the tensors can relate to ultrasound patterns corresponding to the nervous system of the subject. These data structures may include vectors (one-dimensional arrays), traditional matrices (two-dimensional arrays), three-dimensional arrays, multi-dimensional tensors, sparse matrices, compressed representations, hierarchical data structures, graph-based representations, time-series arrays, frequency-domain representations, wavelet decompositions, statistical distributions, probability density functions, feature maps, embedding spaces, dimensionality-reduced representations, manifolds, latent variable spaces, point clouds, voxel grids, octrees, mesh structures, or any combination thereof. The neural data structures may further represent different types of neural information, including spatial coordinates, temporal dynamics, frequency characteristics, phase relationships, correlation structures, connectivity patterns, network topologies, activation patterns, inhibition patterns, hemodynamic responses, metabolic activities, electrical potentials, receptor distributions, neurotransmitter concentrations, structural features, tissue properties, vascular arrangements, white matter tracts, or any other measurable or derivable neural properties. The neural data structures may be pre-processed, filtered, normalized, standardized, transformed, compressed, encrypted, error-corrected, noise-reduced, motion-compensated, artifact-removed, temporally or spatially aligned, interpolated, extrapolated, segmented, clustered, classified, or otherwise algorithmically manipulated to enhance their utility for subsequent analysis, visualization, or modulation purposes. Implementation of these data structures may use any suitable computational representation including fixed-point or floating-point numeric formats, binary encodings, quantized representations, or specialized data types optimized for neural data processing or machine learning operations. The one or more neural data structures can encompass spatial or temporal attributes, in addition to channel data, such as optical channel data. The one or more neural data structures can be dense neural data structures, or sparse neural data structures. The sparse neural data structures can store only voxels containing relevant neural information using coordinate-value pairs, and can be understood to be a sparse matrix or a sparse tensor. Sparse neural data structures can include sparse coordinate formats, indexed voxel collections, or point cloud representations. The one or more neural data structures can include radiofrequency (RF) data, in-phase quadrature (IQ) data, processed RF data, or processed IQ data. The one or more neural data structures can comprise data structures derived from ultrasound waves reflected, e.g., scattered, from the nervous system of the subject and received by the ultrasound transducer.

At 808 in FIG. 8 or 908 in FIG. 9, the first plurality is decoded, via an instruction-determining machine learning algorithm, into first instructions.

At 810 in FIG. 8 or 910 in FIG. 9, the first ongoing psychophysical state of the subject is modulated, based on the first instructions.

At 812 in FIG. 8 or 912 in FIG. 9, the first ongoing psychophysical state of the subject is modulated, based on the first instructions.

Disclosed herein is a method for modulating a second ongoing psychophysical state of the subject, further comprising: emitting second ultrasound waves to the subject, from an ultrasound transducer corresponding to the ultrasound transducer of any of the embodiments herein, to the one or more portions of the nervous system; receiving second sono-enhanced ultrasound waves from the subject, wherein the second sono-enhanced ultrasound waves comprise the second emitted ultrasound waves enhanced via the sono-enhancing contrast agent; determining a second plurality of one or more neural data structures, using one or more processors, based on the second received sono-enhanced ultrasound waves; decoding the second plurality, via an instruction-determining machine learning algorithm, into second instructions; and modulating the second ongoing psychophysical state of the subject, based on the second instructions.

At 914 in FIG. 9, second ultrasound waves are emitted to the subject, from the ultrasound transducer, to one or more portions of the nervous system.

At 916 in FIG. 9, second sono-enhanced ultrasound waves from the subject are received, wherein the second sono-enhanced ultrasound waves comprise the second emitted ultrasound waves enhanced via the sono-enhancing contrast agent.

At 918 in FIG. 9, a second plurality of one or more neural data structures is determined, using one or more processors, based on the second received sono-enhanced ultrasound waves.

At 920 in FIG. 9, the second plurality is decoded, via an instruction-determining machine learning algorithm, into second instructions.

At 922 in FIG. 9, the second ongoing psychophysical state of the subject is modulated, based on the second instructions.

### Methods of Decoding Latent Neural States

Traditional ultrasound imaging methods employ a beamforming process to transform received radiofrequency (RF) data from transducer elements into a spatial representation, such as a B-mode image. The spatial representation is then typically used as an intermediate step for any subsequent analysis, such as for detecting neural activity. The beamforming process is computationally intensive. It scales as a function of the number of voxels in the beamformed image times the number of receiver elements, which can easily result in tens of billions of operations for each beamformed image. For a brain-computer interface, the beamforming process may not be necessary, because the ultimate goal of such an interface oftentimes is to extract underlying latent variables rather than to produce images for human interpretation. That is, traditional ultrasound imaging methods often attempt to extract latent variables, such as latent neural states, from human-interpretable images, and such images are the result of computationally expensive processes, such as beamforming. Rather than using a computationally expensive process such as beamforming, to then derive latent neural states, the methods disclosed herein describe a method that can bypass the computationally expensive beamforming process, and instead, directly interpret non-beamformed data in terms of the latent neural states.

The traditional beamforming approach is especially problematic in the context of brain-machine interfaces. Deployable brain-machine interfaces benefit from real-time latencies. Therefore, the computational overhead introduced by beamforming processes is prohibitive for brain-machine interfaces. Beamforming—such as delay-and-sum (DAS) beamforming algorithms—is mathematically intensive. For each pixel in the output image, the algorithm must calculate precise delays for every transducer element, perform interpolation along hyperbolic paths in the radiofrequency (RF) data, and then sum all the values. The result is a time complexity of O(M×N×q), where M is the number of output pixels, N is the number of transducer elements, and q is the interpolation complexity. The computational burden, as indicated by the extremely high time complexity, becomes prohibitive for real-time applications, and would likely require specialized hardware acceleration. For implantable neural interface systems in particular, the computational overhead would result in prohibitive power consumption, because the consumed power would be directly realized as thermal elevation in the implant.

Beamforming processes, such as DAS beamforming, also inherently discards valuable phase information. When the DAS beamforming algorithm performs envelope detection and compression, key aspects of the phase information are removed. Critical RF data characteristics that may directly correlate with latent neural states are lost during the beamforming process. Subsequent processing of the beamformed images to extract any latent neural states would further compound the information loss. In addition, for brain-computer interfaces and closed-loop neural systems, the beamforming pipeline can introduce unacceptable latencies—typically on the order of 10 ms to 1000 ms. The time required to calculate appropriate delays, interpolations, and envelope-detected images create a fundamental bottleneck that limits the temporal resolution of neural state detection. In addition, the proper implementation of beamforming algorithms requires careful optimization of multiple parameters across various media, such as the f-number, the received aperture, and the speed of sound. The need for continual parameter adjustment introduces additional complexity and error potential in neural monitoring applications. The traditional processing pipeline for beamforming is also very complex. The pipeline typically begins with RF data, which are then hyperbolically delayed and summed, which are then envelope detected, which are then image formed, which are then feature extracted, from which latent neural states can finally be estimated. Each computation in the processing pipeline introduces its own assumptions and approximations. Moreover, the potential for error propagation increases, as the number of computations in the processing pipeline increases.

The methods disclosed herein allow for the direct mapping of RF data to latent neural states, without the beamforming process. The described methods include establishing a direct pathway from the raw RF data that are received by ultrasound transducer elements, to the underlying neural activity patterns, e.g., the latent neural states. In doing so, the methods disclosed herein entirely bypass the conventional spatial reconstruction process. In brief, the methods disclosed herein can include defining N time-varying latent neural states that represent the underlying neural activity patterns, e.g., the latent neural states. Then, rather than converting the RF data to a spatial representation and then to the latent neural states, the approach can create a direct mapping, e.g., a decoding, from the RF data to the latent neural states. The decoding can employ linear regression techniques, such as ridge regression or partial least square regression, to establish the relationship between RF data and latent neural states. The use of linear regression techniques, as an alternative to complex deep learning approaches, for successful decoding, demonstrates the computational efficiency capable of the methods disclosed herein.

Disclosed herein is a method of decoding latent neural states directly from one or more neural data structures, comprising: determining, using one or more processors, the one or more target latent neural states; receiving first ultrasound waves reflected from one or more portions of a nervous system from a subject, at the ultrasound transducer corresponding to any of the embodiments herein; determining one or more first neural data structures from the ultrasound transducer; and decoding the one or more first neural data structures, via a regressor, using the one or more processors, into the one or more target latent neural states, thereby bypassing a beamforming transformation of the one or more neural data structures.

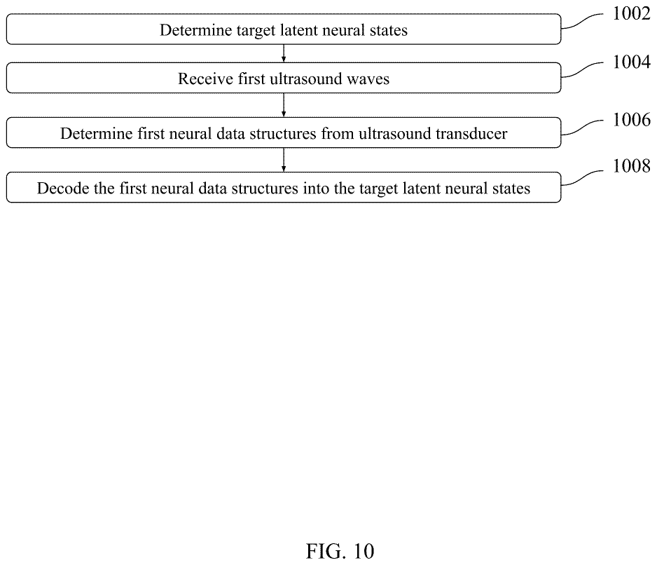

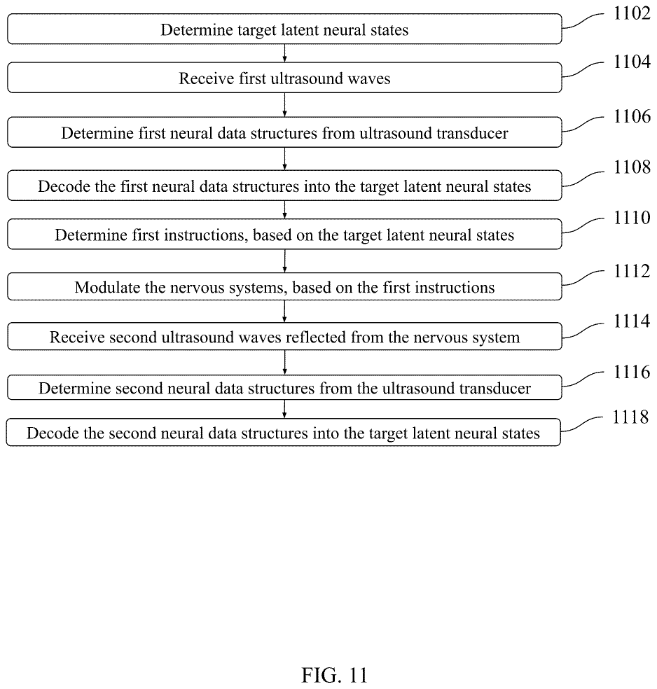

At 1002 in FIG. 10 or at 1102 in FIG. 11, the one or more target latent neural states are determined, using one or more processors. The one or more target latent neural states can be understood as a lower-dimensional representation of neural activity, relative to some input data structure that may represent neural activity, such as radiofrequency (RF) data or in-phase quadrature (IQ) data. The target latent neural state can correspond to a discrete non-neural activity event, such as a particular behavior of a subject.

At 1004 in FIG. 10 or at 1104 in FIG. 11, first ultrasound waves reflected from one or more portions of a nervous system from a subject are received, at the ultrasound transducer corresponding to any of the embodiments herein.

At 1006 in FIG. 10 or at 1106 in FIG. 11, one or more first neural data structures from the ultrasound transducer are determined, using the one or more processors. The one or more first neural data structures can be any data structure that can be represented as one or more arrays or collections of data points. For example, the one or more first neural data structures can comprise tensors, where the tensors can relate to ultrasound patterns corresponding to the nervous system of the subject. These data structures may include vectors (one-dimensional arrays), traditional matrices (two-dimensional arrays), three-dimensional arrays, multi-dimensional tensors, sparse matrices, compressed representations, hierarchical data structures, graph-based representations, time-series arrays, frequency-domain representations, wavelet decompositions, statistical distributions, probability density functions, feature maps, embedding spaces, dimensionality-reduced representations, manifolds, latent variable spaces, point clouds, voxel grids, octrees, mesh structures, or any combination thereof. The first neural data structures may further represent different types of neural information, including spatial coordinates, temporal dynamics, frequency characteristics, phase relationships, correlation structures, connectivity patterns, network topologies, activation patterns, inhibition patterns, hemodynamic responses, metabolic activities, electrical potentials, receptor distributions, neurotransmitter concentrations, structural features, tissue properties, vascular arrangements, white matter tracts, or any other measurable or derivable neural properties. The first neural data structures may be pre-processed, filtered, normalized, standardized, transformed, compressed, encrypted, error-corrected, noise-reduced, motion-compensated, artifact-removed, temporally or spatially aligned, interpolated, extrapolated, segmented, clustered, classified, or otherwise algorithmically manipulated to enhance their utility for subsequent analysis, visualization, or modulation purposes. Implementation of these data structures may use any suitable computational representation including fixed-point or floating-point numeric formats, binary encodings, quantized representations, or specialized data types optimized for neural data processing or machine learning operations. The one or more first neural data structures can encompass spatial or temporal attributes, in addition to channel data, such as optical channel data. The one or more first neural data structures can be dense neural data structures, or sparse neural data structures. The sparse neural data structures can store only voxels containing relevant neural information using coordinate-value pairs, and can be understood to be a sparse matrix or a sparse tensor. Sparse neural data structures can include sparse coordinate formats, indexed voxel collections, or point cloud representations. The one or more first neural data structures can include radiofrequency (RF) data, in-phase quadrature (IQ) data, processed RF data, or processed IQ data. The one or more first neural data structures can comprise data structures derived from ultrasound waves reflected, e.g., scattered, from the nervous system of the subject and received by the ultrasound transducer.

At 1008 in FIG. 10 or at 1108 in FIG. 11, the one or more first neural data structures are decoded, via a regressor, using the one or more processors, into the one or more target latent neural states, thereby bypassing a beamforming transformation of the one or more neural data structures. The regressor can comprise a linear regressor. In some embodiments, the linear regressor can comprise an ordinary least squares regressor, a ridge regressor, a lasso regressor, an elastic net regressor, a partial least squares regressor, a principal component regressor, a weighted least squares regressor, or a least angle regressor. In any of the embodiments herein, the regressor can comprise a non-linear regressor. In some embodiments, the non-linear regressor can comprise a polynomial regressor, a decision tree regressor, a random forest regressor, a logistic regressor, a support vector regressor with non-linear kernels, a neural network, a Gaussian process, a k-nearest neighbor regressor, a spline regressor, kernel ridge regressor, a generalized additive model, a multivariate adaptive regression spline regressor, a kernel regressor, a local regressor, a radial basis function network, an isotonic regressor, a projection pursuit regressor, a quantile regression forest, a sequential state estimator, a Kalman filter, or a multi-task learning regressor. Sparse regressors can include the use of LASSO or elastic net regressions to identify minimal subsets of radiofrequency (RF) or in-phase or quadrature (IQ) features needed for predicting the latent neural states. Sparse regression techniques are advantageous because they reduce computational load, and they can identify the most informative transducer elements. The use of kernel methods can include the application of kernel regression (e.g., kernel ridge regression) to capture non-linear relationships between the RF or IQ data and the latent neural states. The use of kernel methods are advantageous because they can provide higher accuracy for complex neural state mappings, while still maintaining acceptable computational efficiency. The sequential state estimation can include the incorporating of temporal dynamics using Kalman filtering, or some other state-space model. The advantages of sequential state estimation includes improved accuracy, because sequential state estimation involves modeling the temporal evolution of neural states, such as the latent neural states. Multi-task learning methods can involve simultaneously mapping the RF or IQ data to multiple related latent neural state variables. The advantages of multi-task learning include improved robustness, because of shared feature learning across related latent neural states.

The one or more neural data structures can comprise radiofrequency (RF) data, in-phase or quadrature (IQ) data, or an envelope of the radiofrequency data. That is, instead of decoding the latent neural states directly from the RF data, the latent neural states can be decoded directly from the IQ data. The advantages of decoding the latent neural states from IQ data include the lower bandwidth requirements relative to RF data, because IQ data is often downsampled 4×-10×, relative to RF data. The IQ data, however, still maintains phase information critical for discriminating latent neural states. Using the IQ data can involve applying linear regression approaches identical or similar to the linear regression approaches used for RF data, except that the IQ data can include complex-valued data, such that both the magnitude and the phase are potentially included as separate input features. Alternatively, the envelope of RF data—without spatial beamforming—can be used to directly decode the latent neural states. Using the envelope of RF data rather than the RF data itself can provide advantages, such as reduced sensitivity to phase variations, and potentially more robust performance against minor timing jitter.

Disclosed herein is a method of decoding latent neural states directly from the one or more neural data structures, further comprising: determining first instructions, using one or more processors, based on the one or more target latent neural states; modulating the one or more portions of the nervous system of the subject, based on the first instructions; receiving second ultrasound waves reflected from the one or more portions of the nervous system from the subject, at the ultrasound transducer; determining one or more second neural data structures from the ultrasound transducer; and decoding the one or more second neural data structures, via the regressor, using the one or more processors, into the one or more target latent neural states, thereby bypassing the beamforming transformation of the one or more neural data structures.

At 1110 in FIG. 11, the first instructions are determined, using the one or more processors, based on the one or more target latent neural states. The one or more target latent neural states can be understood as a lower-dimensional representation of neural activity, relative to some input data structure that may represent neural activity, such as radiofrequency (RF) data or in-phase quadrature (IQ) data. The target latent neural state can correspond to a discrete non-neural activity event, such as a particular behavior of a subject.

At 1112 in FIG. 11, the one or more portions of the nervous system of the subject are modulated, based on the first instructions.

At 1114 in FIG. 11, second ultrasound waves reflected from the one or more portions of the nervous system from the subject are received, at the ultrasound transducer.

At 1116 in FIG. 11, one or more second neural data structures from the ultrasound transducer are determined, using the one or more processors. The one or more second neural data structures can be any data structure that can be represented as one or more arrays or collections of data points. For example, the one or more second neural data structures can comprise tensors, where the tensors can relate to ultrasound patterns corresponding to the nervous system of the subject. These data structures may include vectors (one-dimensional arrays), traditional matrices (two-dimensional arrays), three-dimensional arrays, multi-dimensional tensors, sparse matrices, compressed representations, hierarchical data structures, graph-based representations, time-series arrays, frequency-domain representations, wavelet decompositions, statistical distributions, probability density functions, feature maps, embedding spaces, dimensionality-reduced representations, manifolds, latent variable spaces, point clouds, voxel grids, octrees, mesh structures, or any combination thereof. The second neural data structures may further represent different types of neural information, including spatial coordinates, temporal dynamics, frequency characteristics, phase relationships, correlation structures, connectivity patterns, network topologies, activation patterns, inhibition patterns, hemodynamic responses, metabolic activities, electrical potentials, receptor distributions, neurotransmitter concentrations, structural features, tissue properties, vascular arrangements, white matter tracts, or any other measurable or derivable neural properties. The second neural data structures may be pre-processed, filtered, normalized, standardized, transformed, compressed, encrypted, error-corrected, noise-reduced, motion-compensated, artifact-removed, temporally or spatially aligned, interpolated, extrapolated, segmented, clustered, classified, or otherwise algorithmically manipulated to enhance their utility for subsequent analysis, visualization, or modulation purposes. Implementation of these data structures may use any suitable computational representation including fixed-point or floating-point numeric formats, binary encodings, quantized representations, or specialized data types optimized for neural data processing or machine learning operations. The one or more neural data structures can encompass spatial or temporal attributes, in addition to channel data, such as optical channel data. The one or more second neural data structures can be dense neural data structures, or sparse neural data structures. The sparse neural data structures can store only voxels containing relevant neural information using coordinate-value pairs, and can be understood to be a sparse matrix or a sparse tensor. Sparse neural data structures can include sparse coordinate formats, indexed voxel collections, or point cloud representations. The one or more second neural data structures can include radiofrequency (RF) data, in-phase quadrature (IQ) data, processed RF data, or processed IQ data. The one or more second neural data structures can comprise data structures derived from ultrasound waves reflected, e.g., scattered, from the nervous system of the subject and received by the ultrasound transducer.

At 1118 in FIG. 11, the one or more second neural data structures are decoded, via the regressor, using the one or more processors, into the one or more target latent neural states, thereby bypassing the beamforming transformation of the one or more neural data structures. The regressor can comprise a linear regressor. In some embodiments, the linear regressor can comprise an ordinary least squares regressor, a ridge regressor, a lasso regressor, an elastic net regressor, a partial least squares regressor, a principal component regressor, a weighted least squares regressor, or a least angle regressor. In any of the embodiments herein, the regressor can comprise a non-linear regressor. In some embodiments, the non-linear regressor can comprise a polynomial regressor, a decision tree regressor, a random forest regressor, a logistic regressor, a support vector regressor with non-linear kernels, a neural network, a Gaussian process, a k-nearest neighbor regressor, a spline regressor, kernel ridge regressor, a generalized additive model, a multivariate adaptive regression spline regressor, a kernel regressor, a local regressor, a radial basis function network, an isotonic regressor, a projection pursuit regressor, a quantile regression forest, a sequential state estimator, a Kalman filter, or a multi-task learning regressor. Sparse regressors can include the use of LASSO or elastic net regressions to identify minimal subsets of radiofrequency (RF) or in-phase or quadrature (IQ) features needed for predicting the latent neural states. Sparse regression techniques are advantageous because they reduce computational load, and they can identify the most informative transducer elements. The use of kernel methods can include the application of kernel regression (e.g., kernel ridge regression) to capture non-linear relationships between the RF or IQ data and the latent neural states. The use of kernel methods are advantageous because they can provide higher accuracy for complex neural state mappings, while still maintaining acceptable computational efficiency. The sequential state estimation can include the incorporating of temporal dynamics using Kalman filtering, or some other state-space model. The advantages of sequential state estimation includes improved accuracy, because sequential state estimation involves modeling the temporal evolution of neural states, such as the latent neural states. Multi-task learning methods can involve simultaneously mapping the RF or IQ data to multiple related latent neural state variables. The advantages of multi-task learning include improved robustness, because of shared feature learning across related latent neural states.

Disclosed herein is a method of decoding latent neural states directly from one or more neural data structures, comprising: determining, using one or more processors, the one or more target latent neural states; receiving first ultrasound waves reflected from one or more portions of a nervous system from a subject, at the ultrasound transducer corresponding to any of the embodiments herein; determining one or more first neural data structures from the ultrasound transducer; transforming the one or more first neural data structures into a first non-beamformed intermediate representation; and decoding the first non-beamformed intermediate representation, via a regressor, using the one or more processors, into the one or more target latent neural states, thereby bypassing a beamforming transformation of the first non-beamformed intermediate representation.

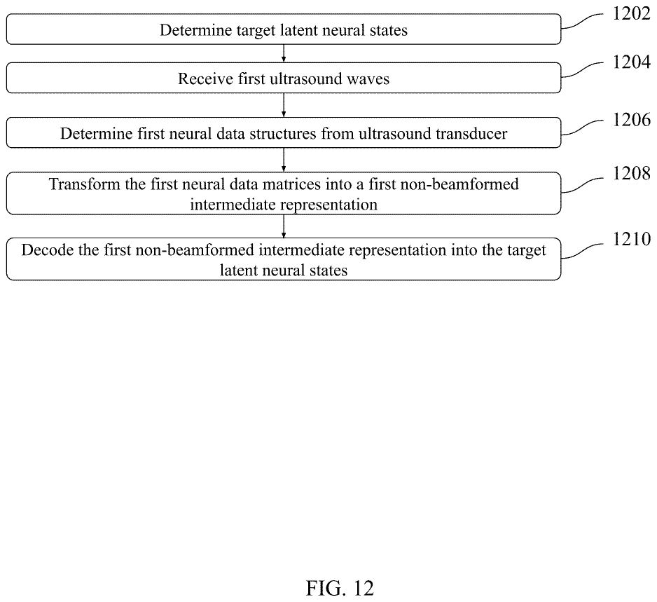

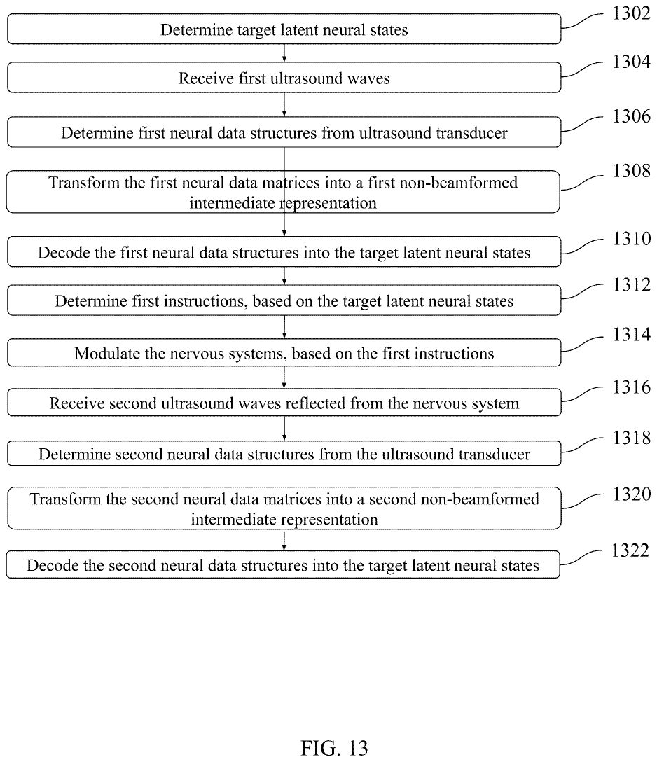

At 1202 in FIG. 12 or at 1302 in FIG. 13, the one or more target latent neural states are determined, using one or more processors. The one or more target latent neural states can be understood as a lower-dimensional representation of neural activity, relative to some input data structure that may represent neural activity, such as radiofrequency (RF) data or in-phase quadrature (IQ) data. The target latent neural state can correspond to a discrete non-neural activity event, such as a particular behavior of a subject.

At 1204 in FIG. 12 or at 1304 in FIG. 13, first ultrasound waves reflected from one or more portions of a nervous system from a subject are received, at the ultrasound transducer corresponding to any of the embodiments herein.

At 1206 in FIG. 12 or at 1306 in FIG. 13, one or more first neural data structures from the ultrasound transducer are determined, using the one or more processors.

At 1208 in FIG. 12 or at 1308 in FIG. 13, the one or more first neural data structures into a first non-beamformed intermediate representation are transformed, using the one or more processors. The first non-beamformed intermediate representation can comprise various transformation methods, including registration or alignment methods. For example, the transformation method can include wavelet decomposition mapping. In wavelet decomposition mapping, wavelet transforms can be applied to radiofrequency (RF) or in-phase or quadrature (IQ) data, before applying a regression. The advantages of performing a wavelet decomposition mapping include the extracting of multi-scale features from the data, noise reduction, and the ability to focus on specific frequency bands that may be particularly relevant to neural activity. The transformation method can include time-frequency representations. In time-frequency representations, a time-frequency representation, such as a short-time Fourier transform, can be mapped to the latent neural states. The advantages of using time-frequency representations include the capturing of spectral shifts that may be associated with different latent neural states. The transformation method can include compressed domain processing, e.g., dimensionality reduction. That is, random projections via some dimensionality reduction technique can be applied to the RF or the IQ data, and then the data can be mapped via some other transformation, or directly to the one or more latent neural states. Compressed domain processing is advantageous because it further reduces computational overhead, and because it potentially immunizes the processing from noise in the inputs. The transformation methods can include registration or alignment methods. That is, the first non-beamformed representation can comprise a registered representation. The registered representation can comprise a motion compensated representation, a spatiotemporal registered representation, or an adaptively sampled representation. The motion compensated representation can incorporate tissue motion estimations directly into the processing pipeline. For example, the motion compensated representation can estimate and compensate for bulk tissue motion prior to performing the regression for the neural latent states. Motion compensated representations are advantageous in that they provide improved robustness in moving tissue, e.g., against tissue that moves due to respiration or pulsation. Spatiotemporal registered representations can include registering RF or IQ data patterns across time points to a common reference frame. Advantages of spatiotemporal registered representations include reducing effects from probe movement or subject movement, which can provide confounding effects on estimating latent neural states. Adaptively sampled representations can include dynamically adjusting the representation to parts of the RF or IQ data that contribute most to the latent neural state estimation. Implementing the adaptive sampling for the adaptively sampled representations can include learning the weights that emphasize informative timepoints or channels from the RF or IQ data. Adaptively sampled representations are advantageous in that they reduce computational load, by focusing on the most relevant signal components.

At 1210 in FIG. 12 or at 1310 in FIG. 13, the first non-beamformed intermediate representation is decoded, via a regressor, using the one or more processors, into the one or more target latent neural states, thereby bypassing a beamforming transformation of the first non-beamformed intermediate representation. The regressor can comprise a linear regressor. In some embodiments, the linear regressor can comprise an ordinary least squares regressor, a ridge regressor, a lasso regressor, an elastic net regressor, a partial least squares regressor, a principal component regressor, a weighted least squares regressor, or a least angle regressor. In any of the embodiments herein, the regressor can comprise a non-linear regressor. In some embodiments, the non-linear regressor can comprise a polynomial regressor, a decision tree regressor, a random forest regressor, a logistic regressor, a support vector regressor with non-linear kernels, a neural network, a Gaussian process, a k-nearest neighbor regressor, a spline regressor, kernel ridge regressor, a generalized additive model, a multivariate adaptive regression spline regressor, a kernel regressor, a local regressor, a radial basis function network, an isotonic regressor, a projection pursuit regressor, a quantile regression forest, a sequential state estimator, a Kalman filter, or a multi-task learning regressor. Sparse regressors can include the use of LASSO or elastic net regressions to identify minimal subsets of radiofrequency (RF) or in-phase or quadrature (IQ) features needed for predicting the latent neural states. Sparse regression techniques are advantageous because they reduce computational load, and they can identify the most informative transducer elements. The use of kernel methods can include the application of kernel regression (e.g., kernel ridge regression) to capture non-linear relationships between the RF or IQ data and the latent neural states. The use of kernel methods are advantageous because they can provide higher accuracy for complex neural state mappings, while still maintaining acceptable computational efficiency. The sequential state estimation can include the incorporating of temporal dynamics using Kalman filtering, or some other state-space model. The advantages of sequential state estimation includes improved accuracy, because sequential state estimation involves modeling the temporal evolution of neural states, such as the latent neural states. Multi-task learning methods can involve simultaneously mapping the RF or IQ data to multiple related latent neural state variables. The advantages of multi-task learning include improved robustness, because of shared feature learning across related latent neural states.

Disclosed herein is a method of decoding latent neural states directly from the one or more neural data structures, further comprising: determining first instructions, using one or more processors, based on the one or more target latent neural states; modulating the one or more portions of the nervous system of the subject, based on the first instructions; receiving second ultrasound waves reflected from the one or more portions of the nervous system from the subject, at the ultrasound transducer; determining one or more second neural data structures from the ultrasound transducer; transforming the one or more second neural data structures into a second non-beamformed intermediate representation; and decoding the second non-beamformed intermediate representation, via a regressor, using the one or more processors, into the one or more target latent neural states, thereby bypassing a beamforming transformation of the second non-beamformed intermediate representation.

At 1312 in FIG. 13, first instructions are determined, using the one or more processors, based on the one or more target latent neural states. The one or more target latent neural states can be understood as a lower-dimensional representation of neural activity, relative to some input data structure that may represent neural activity, such as radiofrequency (RF) data or in-phase quadrature (IQ) data. The target latent neural state can correspond to a discrete non-neural activity event, such as a particular behavior of a subject.

At 1314 in FIG. 13, the one or more portions of the nervous system of the subject are modulated, based on the first instructions.

At 1316 in FIG. 13, second ultrasound waves reflected from the one or more portions of the nervous system from the subject are received, at the ultrasound transducer.

At 1318 in FIG. 13, one or more second neural data structures from the ultrasound transducer are determined, using the one or more processors.

At 1320 in FIG. 13, the one or more second neural data structures are transformed into a second non-beamformed intermediate representation, using the one or more processors. The second non-beamformed intermediate representation can comprise various transformation methods, including registration or alignment methods. For example, the transformation method can include wavelet decomposition mapping. In wavelet decomposition mapping, wavelet transforms can be applied to radiofrequency (RF) or in-phase or quadrature (IQ) data, before applying a regression. The advantages of performing a wavelet decomposition mapping include the extracting of multi-scale features from the data, noise reduction, and the ability to focus on specific frequency bands that may be particularly relevant to neural activity. The transformation method can include time-frequency representations. In time-frequency representations, a time-frequency representation, such as a short-time Fourier transform, can be mapped to the latent neural states. The advantages of using time-frequency representations include the capturing of spectral shifts that may be associated with different latent neural states. The transformation method can include compressed domain processing, e.g., dimensionality reduction. That is, random projections via some dimensionality reduction technique can be applied to the RF or the IQ data, and then the data can be mapped via some other transformation, or directly to the one or more latent neural states. Compressed domain processing is advantageous because it further reduces computational overhead, and because it potentially immunizes the processing from noise in the inputs. The transformation methods can include registration or alignment methods. That is, the second non-beamformed representation can comprise a registered representation. The registered representation can comprise a motion compensated representation, a spatiotemporal registered representation, or an adaptively sampled representation. The motion compensated representation can incorporate tissue motion estimations directly into the processing pipeline. For example, the motion compensated representation can estimate and compensate for bulk tissue motion prior to performing the regression for the neural latent states. Motion compensated representations are advantageous in that they provide improved robustness in moving tissue, e.g., against tissue that moves due to respiration or pulsation. Spatiotemporal registered representations can include registering RF or IQ data patterns across time points to a common reference frame. Advantages of spatiotemporal registered representations include reducing effects from probe movement or subject movement, which can provide confounding effects on estimating latent neural states. Adaptively sampled representations can include dynamically adjusting the representation to parts of the RF or IQ data that contribute most to the latent neural state estimation. Implementing the adaptive sampling for the adaptively sampled representations can include learning the weights that emphasize informative timepoints or channels from the RF or IQ data. Adaptively sampled representations are advantageous in that they reduce computational load, by focusing on the most relevant signal components.

At 1322 in FIG. 13, the second non-beamformed intermediate representation, via a regressor, is decoded, using the one or more processors, into the one or more target latent neural states, thereby bypassing a beamforming transformation of the second non-beamformed intermediate representation. The regressor can comprise a linear regressor. In some embodiments, the linear regressor can comprise an ordinary least squares regressor, a ridge regressor, a lasso regressor, an elastic net regressor, a partial least squares regressor, a principal component regressor, a weighted least squares regressor, or a least angle regressor. In any of the embodiments herein, the regressor can comprise a non-linear regressor. In some embodiments, the non-linear regressor can comprise a polynomial regressor, a decision tree regressor, a random forest regressor, a logistic regressor, a support vector regressor with non-linear kernels, a neural network, a Gaussian process, a k-nearest neighbor regressor, a spline regressor, kernel ridge regressor, a generalized additive model, a multivariate adaptive regression spline regressor, a kernel regressor, a local regressor, a radial basis function network, an isotonic regressor, a projection pursuit regressor, a quantile regression forest, a sequential state estimator, a Kalman filter, or a multi-task learning regressor. Sparse regressors can include the use of LASSO or elastic net regressions to identify minimal subsets of radiofrequency (RF) or in-phase or quadrature (IQ) features needed for predicting the latent neural states. Sparse regression techniques are advantageous because they reduce computational load, and they can identify the most informative transducer elements. The use of kernel methods can include the application of kernel regression (e.g., kernel ridge regression) to capture non-linear relationships between the RF or IQ data and the latent neural states. The use of kernel methods are advantageous because they can provide higher accuracy for complex neural state mappings, while still maintaining acceptable computational efficiency. The sequential state estimation can include the incorporating of temporal dynamics using Kalman filtering, or some other state-space model. The advantages of sequential state estimation includes improved accuracy, because sequential state estimation involves modeling the temporal evolution of neural states, such as the latent neural states. Multi-task learning methods can involve simultaneously mapping the RF or IQ data to multiple related latent neural state variables. The advantages of multi-task learning include improved robustness, because of shared feature learning across related latent neural states.

The methods described herein articulate a transformative shift in ultrasound signal processing for neural interfaces, with several key technical advantages. For one, inferring latent neural states directly from non-beamformed data provides immense improvements in computational efficiency. By eliminating the beamforming step, the methods described herein dramatically reduces computational complexity. That is, the methods described herein reduce the computational complexity from O(M×N×q) to O(N×d), where M is the number of output voxels, which is often on the order of a million for volumetric data, N is the number of transducer elements, which is often on the order of thousands, q is the interpolation factor, and d is the dimensionality of the latent space, which is often small. The reduced computational complexity from O(M×N×q) to O(N×d) marks orders of magnitude improvement in computational requirements. In addition, the methods disclosed herein can eliminate complex beamforming calculations, such as delay-and-sum (DAS) beamforming calculations. The methods described herein completely bypass the need to calculate precise time-of-flight hyperbolic paths for each pixel or look-up table. Such calculations comprise the most computationally intensive aspects of DAS beamforming. The direct mapping of non-beamformed representations also significantly reduces processing time from a minimum of tens of milliseconds to potentially sub-millisecond latencies. By eliminating the entire beamforming pipeline and associated buffer requirements, the system can operate at biologically relevant timescales (1-10 ms), allowing for truly closed-loop neural interfaces. The methods disclosed herein also preserve key information. For example, envelope detection and log compression preserves phase information and subtle radiofrequency data signatures that are critical for neural state discrimination, but are typically discarded during beamforming processes. The methods described herein captures all relevant signal characteristics during regression, e.g., during linear regression. In addition, by mapping directly to the latent neural state space, the methods described herein optimize for the dimensionality that matters—the latent neural state space—rather than wasting resources on high spatial resolution that does not contribute to the decoding of the latent neural state space. The methods described herein also eliminates the need for carefully tuning multiple beamforming parameters across various media, such as speed of sound, f-number, aperture size, etc., all of which typically require expert knowledge and application-specific optimization. The methods described herein also allow for hardware implementations that can directly process radiofrequency data without specialized beamforming circuits, potentially leading to more compact, power-efficient neural interfaces that could be implemented on standard processing units rather than specialized hardware. The methods described herein are also highly adaptable: they can adapt to various transducer configurations and can be retrained for different latent neural state detection tasks, without requiring re-optimization of the beamforming process.

The methods of inferring latent neural states described herein allow for applications that were previously impractical. For example, the methods described herein allow for ultra-fast neural state detection. By removing the beamforming bottleneck, neural state detection can potentially operate at >100 Hz, allowing for the tracking of fast neural dynamics. The methods described herein can also allow for implantable neural interfaces. The reduced computational requirements also make it feasible to implement processing on low-power implantable hardware. In addition, the low latencies accomplished by the methods described herein allow for true closed-loop systems where neural state detection and stimulation can occur within biologically relevant timeframes, such as during perceptibly real-time timeframes (e.g., latencies of approximately 200 ms or less). The simplified processing pipeline introduced by the methods described herein also allow for easier integration of ultrasound-based neural sensing with other modalities, such as electrophysiological (e.g., electroencephalograms) or optical modalities, in real-time. The methods described herein allow for the real-time decoding of latent neural states for control applications, the continuous monitoring of neural activity patterns for scientific or medical purposes, the rapid assessment of tissue states based on underlying biophysical properties, or the real-time feedback during clinical procedures via the direct mapping to relevant state variables.

The methods described herein represent a massive advancement. The direct mapping of RF or IQ data to latent neural states provides many benefits for ultrasound signal processing, especially as they relate to neural interfaces. The methods described herein eliminate the computationally intensive and potentially information-losing beamforming processes, and allows for faster, more efficient, and potentially more accurate latent neural state detection. The methods described herein allows for real-time closed-loop neural interfaces and brain-computer interfaces with drastically reduced latency and computational requirements.

### Computer Systems and Networks

Example systems designed to implement the disclosed methods and systems for imaging and modulating the nervous system of a subject are described below. The systems may comprise, e.g., one or more processors, and a memory unit communicatively coupled to the one or more processors and configured to store instructions that, when executed by the one or more processors, cause the system to perform the disclosed methods (e.g., methods described with respect to FIGS. 3-13).

In some instances, the disclosed systems may further comprise signal post-processing protocols, local data storage servers (e.g., network-attached storages), additional data storage modules, data communication modules (e.g., Bluetooth®, WiFi HaLow, impulse-radio ultra-wideband, ultra-wideband, ultrasonic data transmission, Light Fidelity (Li-Fi), millimeter wave technologies, wireless chip-to-chip, intranet, or internet communication hardware and associated software), display modules, one or more local and/or cloud-based software packages (e.g., remote servers with access to Graphics Processing Units (GPUs) and Central Processing Units (CPUs)), etc., or any combination thereof. In some instances, the systems may comprise, or be part of, a computer system or computer network as described elsewhere herein.

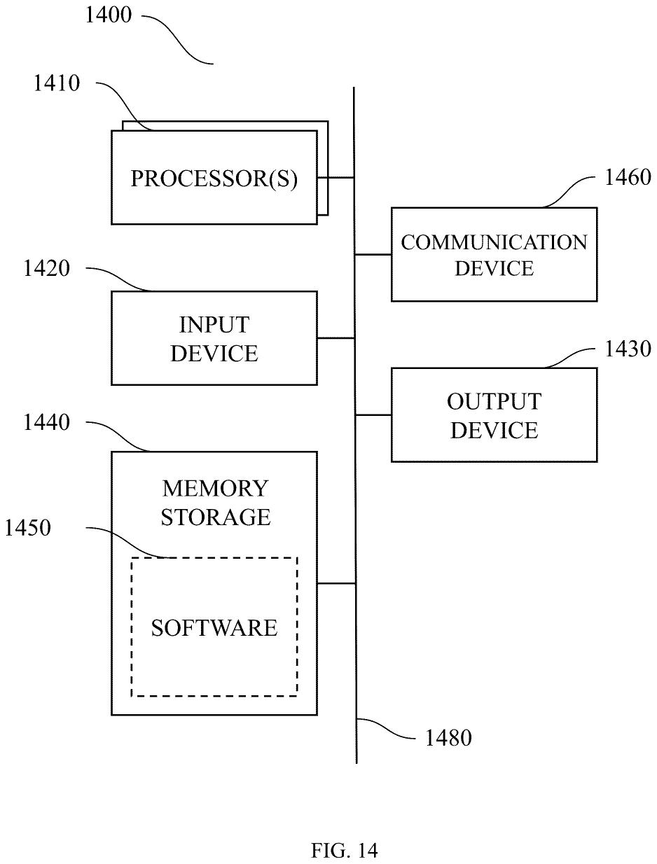

FIG. 14 illustrates an example of a computing device or system in accordance with one embodiment. Device 1400 can be a device for performing the steps described with respect to FIGS. 3-13. For example, device 1400 is a controller coupled to one or more ultrasound transducers, wherein the controller can comprise a power source and a processor. As another example, device 1400 is a device for determining modulation instructions. As another example, device 1400 is a device for processing one or more neural data structures. As another example, device 1400 is a device for communicating one or more neural data structures from one or more ultrasound transducers (e.g., receiving the one or more neural data structures from the transducers, transmitting the one or more neural data structures for determining modulation instructions). As another example, device 1400 is a device for communicating modulation instructions to a system for performing the modulation. As another example, device 1400 is a device for predicting an expected physiological state of the subject.

Device 1400 can be a host computer connected to a network. Device 1400 can be a client computer or a server. As shown in FIG. 14, device 1400 can be any suitable type of microprocessor-based device, such as a personal computer, workstation, server or handheld computing device (portable electronic device) such as a phone or tablet. The device can include, for example, one or more processor(s) 1410, input devices 1420, output devices 1430, memory or storage devices 1440, and communication devices 1460. Software 1450 residing in memory or storage 1440 may comprise, e.g., an operating system as well as software for executing the methods described herein. Input device 1420 and output device 1430 can generally correspond to those described herein and can either be connectable or integrated with the computer.

Input device 1420 can be any suitable device that provides input, such as a touch screen, keyboard or keypad, mouse, or voice-recognition device. The input device 1420 may comprise a user interface for receiving an input to perform a step of the disclosed method. Output device 1430 can be any suitable device that provides output, such as a touch screen, haptics device, or speaker.

Storage 1440 can be any suitable device that provides storage (e.g., an electrical, magnetic or optical memory including a RAM (volatile and non-volatile), cache, hard drive, or removable storage disk). Communication device 1460 can include any suitable device capable of transmitting and receiving signals over a network, such as a network interface chip or device. The components of the computer can be connected in any suitable manner, such as via a wired media (e.g., a physical system bus 1480, Ethernet connection, or any other wire transfer technology) or wirelessly (e.g., Bluetooth®, Wi-Fi®, or any other wireless technology).

Software 1450, which can be stored as executable instructions in storage 1440 and executed by processor(s) 1410, can include, for example, an operating system and/or the processes that embody the functionality of the methods of the present disclosure (e.g., as embodied in the devices as described herein).

Software module 1450 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 1440, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 1450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

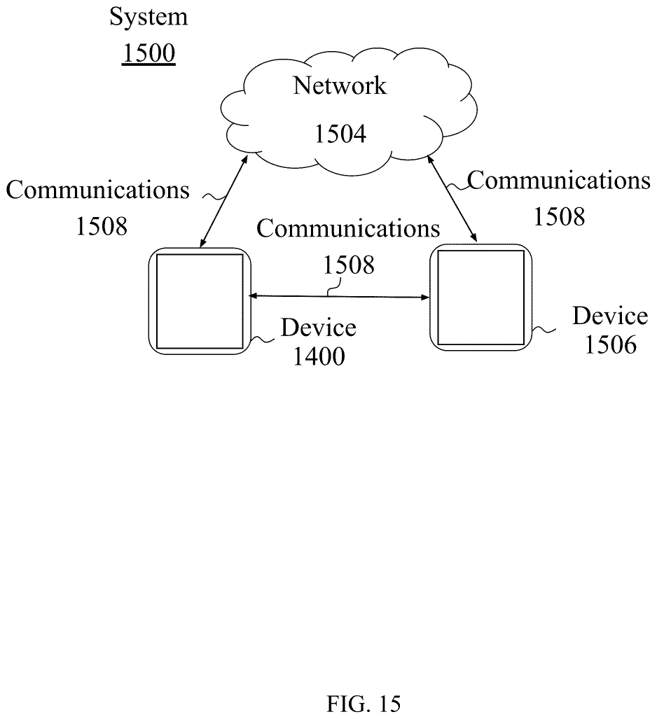

Device 1400 may be connected to a network (e.g., network 1504, as shown in FIG. 15 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1400 can be implemented using any operating system, e.g., an operating system suitable for operating on the network. Software module 1450 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 1410.

FIG. 15 illustrates an example of a computing system. In some embodiments, in system 1100, device 1500 is connected to network 1504, which is also connected to device 5106.

Devices 1400 and 5106 may communicate, e.g., using suitable communication interfaces via network 1504, such as a Local Area Network (LAN), Virtual Private Network (VPN), or the Internet. In some embodiments, network 1504 can be, for example, the Internet, an intranet, a virtual private network, a cloud network, a wired network, or a wireless network. Devices 1400 and 1506 may communicate, in part or in whole, via wireless or hardwired communications, such as Ethernet, IEEE 802.11b wireless, or the like. Additionally, devices 1400 and 1506 may communicate, e.g., using suitable communication interfaces, via a second network, such as a mobile/cellular network. Communication between devices 1400 and 1506 may further include or communicate with various servers such as a mail server, mobile server, media server, telephone server, and the like. In some embodiments, Devices 1400 and 1506 can communicate directly (instead of, or in addition to, communicating via network 1504), e.g., via wireless or hardwired communications, such as Ethernet, IEEE 802.11b wireless, or the like. In some embodiments, devices 1400 and 1506 communicate via communications 1508, which can be a direct connection or can occur via a network (e.g., network 1504).

One or all of devices 1400 and 1506 generally include logic (e.g., http web server logic) or are programmed to format data, accessed from local or remote databases or other sources of data and content, for providing and/or receiving information via network 1504 according to various examples described herein.

### EXEMPLARY EMBODIMENTS

Exemplary implementations of the devices, systems, and methods described herein include:
1. An ultrasound transducer configured to emit ultrasound waves, or receive the ultrasound waves, from one or more portions of a nervous system from a subject comprising a sono-enhancing contrast agent, the ultrasound transducer comprising:
   a sonolucent window;
   an ultrasonic transducer array, wherein
      an element in the ultrasonic transducer array is 20 to 500 microns wide, and
      the ultrasonic transducer array comprises at least 100 elements;
   a control circuit configured to receive or execute instructions for activating a modulating mode or a monitoring mode for the ultrasound transducer; and
   a processing circuit configured to transform the received ultrasound waves into one or more neural data structures.
2. An ultrasound transducer configured to emit ultrasound waves, or receive the ultrasound waves, from one or more portions of a nervous system from a subject comprising a sono-enhancing contrast agent, the ultrasound transducer comprising:
   a sonolucent window;
   an ultrasonic transducer array, wherein
      an element in the ultrasonic transducer array is 20 to 500 microns wide,
      the ultrasonic transducer array comprises at least 100 elements, and
      the ultrasonic transducer array is configured to receive or execute instructions for modulating the one or more portions of the nervous system from the subject; and
   a processing circuit configured to transform the received ultrasound waves into one or more neural data structures.
3. An ultrasound transducer configured to emit ultrasound waves, or receive the ultrasound waves, from one or more portions of a nervous system from a subject comprising a sono-enhancing contrast agent, the ultrasound transducer comprising:
   a sonolucent window;
   an ultrasonic transducer array, wherein
      an element in the ultrasonic transducer array is 20 to 500 microns wide,
      the ultrasonic transducer array comprises at least 100 elements, and
      the ultrasonic transducer array is configured to receive or execute instructions for monitoring the one or more portions of the nervous system from the subject; and
   a processing circuit configured to transform the received ultrasound waves into one or more neural data structures.
4. The ultrasound transducer of any of embodiments 1-3, wherein the one or more portions of the nervous system from the subject comprises a whole brain, a brain region, a neural circuit, or a single neuron.
5. The ultrasound transducer of any of embodiments 1-4, wherein the one or more portions of the nervous system from the subject comprise a dorsolateral prefrontal cortex, frontopolar cortex, anterior cingulate cortex, inferotemporal cortex, visual cortex, auditory cortex, somatosensory cortex, posterior parietal cortex, temporoparietal junction, nucleus accumbens, ventral tegmental area, basal ganglia, orbitofrontal cortex, amygdala, insula, ventromedial prefrontal cortex, hippocampus, entorhinal cortex, parahippcampal gyrus, locus coerulus, reticular formation, ventral striatum, anterior insula, Broca's area, Wernicke's area, medial prefrontal cortex, temporoparietal junction, anterior insula, posterior insula, supplementary motor area, pre-supplementary motor area, or a combination thereof.
6. The ultrasound transducer of any of embodiments 1-5, wherein the one or more neural data structures corresponds to an imaging volume of at most 0.2 cm³, 0.6 cm³, 12 cm³, 100 cm³, 700 cm³ or 1000 cm³.
7. The ultrasound transducer of any of embodiments 1-6, wherein the one or more neural data structures comprises imaging data at an optical resolution.
8. The ultrasound transducer of embodiment 7, wherein the optical resolution corresponds to approximately a width of the element in the ultrasonic transducer array.
9. The ultrasound transducer of embodiment 8, wherein the optical resolution corresponds to approximately the width of the element in the ultrasonic transducer array, based on an approximately one-to-one ratio.
10. The ultrasound transducer of embodiment 8 or 9, wherein the optical resolution is at least 20 microns, 25 microns, 50 microns, 100 microns, 150 microns, 200 microns, 250 microns, 300 microns, or 500 microns.
11. The ultrasound transducer of any of embodiments 1-10, wherein the one or more neural data structures comprises precursor imaging data at a computational superresolution.
12. The ultrasound transducer of embodiment 11, wherein the computational superresolution corresponds to less than the width of the element in the ultrasonic transducer array.
13. The ultrasound transducer of embodiment 11 or 12, wherein the computational superresolution corresponds to approximately the width of the element in the ultrasonic transducer array, based on an approximately multi-to-one ratio.
14. The ultrasound transducer of any of embodiments 1-13, wherein the one or more neural data structures comprises imaging data at an imaging depth.
15. The ultrasound transducer of embodiment 14, wherein the imaging depth is at least 0.04 cm, 0.1 cm, 0.2 cm, 0.4 cm, 0.5 cm, 2.0 cm, 4.0 cm, 6.0 cm, or 8.0 cm.
16.The ultrasound transducer of any of embodiments 1-15, wherein the ultrasound waves comprise a frequency of at most 100 MHz, 60 MHz, 15 MHz, 7.5 MHz, 5.0 MHz, 2.5, or 1.0 MHz.
17. The ultrasound transducer of embodiment 16, wherein the optical resolution corresponds to approximately the frequency.
18. The ultrasound transducer of embodiment 16 or 17, wherein the computational superresolution corresponds to approximately the frequency.
19. The ultrasound transducer of any of embodiments 1-18, wherein the one or more neural data structures comprises hemodynamic data.
20. The ultrasound transducer of any of embodiments 1-19, wherein the one or more neural data structures comprises neural activity data.
21. The ultrasound transducer of any of embodiments 1-20, wherein the one or more neural data structures comprises neural anatomy data.
22. The ultrasound transducer of any of embodiments 1-21, wherein the element in the ultrasonic transducer array is circular, elliptical, square, rectangular, hexagonal, regular, or irregular, in shape.
23. The ultrasound transducer of any of embodiments 1-22, wherein the ultrasonic transducer array is a capacitive micromachined ultrasonic transducer (CMUT) array or a piezoelectric micromachined ultrasonic transducer (PMUT) array.
24. The ultrasound transducer of embodiment 23, wherein the CMUT array comprises a flat membrane CMUT, a curved membrane CMUT, a dual-electrode CMUT, a collapsed-mode CMUT, a wafer-bonded CMUT, a surface-micromachined CMUT, a CMUT-on-CMOS integrated device, a piston-shaped CMUT, an annular array CMUT, or a flexible CMUT.
25. The ultrasound transducer of embodiment 23, wherein the PMUT array comprises a unimorph PMUT, a bimorph PMUT, a dome-shaped PMUT, a suspended membrane PMUT, an aluminum nitride PMUT, a lead zirconate titanate PMUT, a scandium-doped aluminum nitride PMUT, a lithium niobate PMUT, a polyvinylidene fluoride PMUT, a thin-film PMUT, or a flexible PMUT.
26. The ultrasound transducer of any of embodiments 1-25, wherein the ultrasound transducer is an implantable ultrasound transducer.
27. The ultrasound transducer of any of embodiments 1-25, wherein the ultrasound transducer is a non-implantable ultrasound transducer.
28. The ultrasound transducer of embodiment 27, wherein the non-implantable ultrasound transducer comprises a wearable ultrasound transducer.
29. The ultrasound transducer of any of embodiments 1-28, wherein the sono-enhancing contrast agent comprises a biological sono-enhancing contrast agent, a polymeric sono-enhancing contrast agent, or a pharmaceutical sono-enhancing contrast agent.
30. The ultrasound transducer of embodiment 29, wherein the biological sono-enhancing contrast agent comprises a genetically-encoded sono-enhancing contrast agent.
31. The ultrasound transducer of embodiment 30, wherein the genetically-encoded sono-enhancing contrast agent comprises an ultrasonic reporter of calcium, an ultrasonic reporter of voltage, or an ultrasonic reporter of a neurotransmitter.
32. The ultrasound transducer of any of embodiments 1-31, wherein the subject comprises a human subject or a non-human primate subject.
33. The ultrasound transducer of any of embodiments 1-32, wherein the subject has, or is suspected of having, a neural dysfunction.
34. The ultrasound transducer of embodiment 33, wherein the neural dysfunction comprises a motor dysfunction, a sensory dysfunction, a cognitive dysfunction, a neurological condition, or a neuropsychiatric dysfunction.
35. The ultrasound transducer of embodiment 34, wherein the motor dysfunction comprises a spinal cord injury, amyotrophic lateral sclerosis, locked-in syndrome, stroke, Parkinson's disease, or essential tremor.
36. The ultrasound transducer of embodiment 34 or 35, wherein the sensory dysfunction comprises blindness, deafness, epilepsy, a loss of chemosensation, or a loss of somatosensation.
37. The ultrasound transducer of any of embodiments 34-36, wherein the neuropsychiatric dysfunction comprises, clinical depression, schizophrenia, addiction, post-traumatic stress disorder, eating disorder, or sleep disorder.
38. The ultrasound transducer of any of embodiments 1-37, wherein the ultrasound transducer provides a neural augmenting to the subject.
39. The ultrasound transducer of any of embodiments 1-38, wherein the neural augmenting comprises sensorimotor integrating, sensory augmenting, integrating to a virtual reality, integrating to an augmented reality, modulating a brain state, or integrating to an artificial intelligence.
40. A system for monitoring or modulating one or more portions of a nervous system from a subject, comprising:
   one or more ultrasound transducers, an ultrasound transducer of the one or more ultrasound transducers corresponding to the ultrasound transducer of any of embodiments 1-39;
   a controller coupled to each of the one or more ultrasound transducers, the controller comprising a power source and a processor,
      wherein the power source is configured to power each of the one or more ultrasound transducers; and
      wherein the processor is configured to execute a method, the method comprising:
         sending one or more signals to the one or more ultrasound transducers; and
         receiving one or more neural data structures from the one or more ultrasound transducers.
41. The system of embodiment 40, wherein the method further comprises emitting or receiving, via the one or more ultrasound transducers, ultrasound waves.
42. The system of embodiment 40 or 41, wherein the one or more portions of the nervous system from the subject comprises a whole brain, a brain region, a neural circuit, or a single neuron.
43. The system of any of embodiments 40-42, wherein the one or more portions of the nervous system from the subject comprise a dorsolateral prefrontal cortex, frontopolar cortex, anterior cingulate cortex, inferotemporal cortex, visual cortex, auditory cortex, somatosensory cortex, posterior parietal cortex, temporoparietal junction, nucleus accumbens, ventral tegmental area, basal ganglia, orbitofrontal cortex, amygdala, insula, ventromedial prefrontal cortex, hippocampus, entorhinal cortex, parahippcampal gyrus, locus coerulus, reticular formation, ventral striatum, anterior insula, Broca's area, Wernicke's area, medial prefrontal cortex, temporoparietal junction, anterior insula, posterior insula, supplementary motor area, pre-supplementary motor area, or a combination thereof.
44. The system of any of embodiments 40-43, wherein the one or more neural data structures corresponds to an imaging volume of at most 0.2 cm³, 0.6 cm³, 12 cm³, 700 cm³ or 1000 cm³.
45. The system of any of embodiments 40-44, wherein the one or more neural data structures comprises imaging data at an optical resolution.
46. The system of embodiment 44 or 45, wherein the system comprises between one and ten ultrasound transducers.
47. The system of any of embodiments 40-46, the system further comprising:
   a remote hub, wherein
      the remote hub is configured to receive the one or more neural data structures from the controller, and
      the remote hub is configured to transmit external data to the controller.
48. The system of any of embodiments 40-47, wherein the remote hub is configured to communicate with a display to provide a user interface for controlling the system.
49. The system of any of embodiments 40-48, wherein the ultrasound transducer and the controller are configured to communicate wirelessly.
50. The system of embodiment 40-49, wherein the ultrasound transducer and the controller are configured to communicate via Bluetooth, Bluetooth low energy, WiFi, Wi-Fi HaLow, ultra-wideband, impulse-radio ultra-wideband, ultrasonic data transmission, optical data communication, Light Fidelity (li-fi), millimeter wave technologies, wireless chip-to-chip, or a combination thereof.
51. The system of any of embodiments 40-50, wherein the one or more signals are configured to coordinate emission of ultrasound waves via the ultrasound transducer and further configured to coordinate receipt of the ultrasound waves.
52. The system of embodiment 51, wherein the controller comprises a clock, and wherein the one or more signals are sent based on predetermined intervals associated with the clock.
53. The system of embodiment 51, wherein the controller comprises a central clock, and the one or more ultrasound transducers each comprise a peripheral clock, and wherein the one or more signals correspond to reset signals associated with the central clock.
54. The system of any of embodiments 40-53, wherein the controller transforms the one or more neural data structures.
55. The system of any of embodiments 40-54, wherein the controller transmits the one or more neural data structures.
56. The system of any of embodiments 40-55, wherein the subject engages in a clinically relevant behavior while the ultrasound transducer obtains the one or more neural data structures.
57. The system of embodiment 56, wherein the clinically relevant behavior comprises activities of daily living, estimates of movement, motion capture, facial expression and response time, self-reported mood, self-reported cognitive state, heart rate, heart rate variability, breathing rate, oxygenation, galvanic skin response, inertial monitoring, or a combination thereof.
58. The system of any of embodiments 40-57, wherein the system is configured to modify neural activity of the subject based on the one or more neural data structures.
59. The system of embodiment 58, wherein modifying the neural activity of the subject based on the one or more neural data structures occurs in real-time.
60. The system of embodiment 58 or 59, wherein a real-time modifying of the neural activity of the subject comprises a response time of 5 seconds or less, after receiving the one or more neural data structures.
61. The system of embodiment 58-60, wherein the real-time modifying of the neural activity occurs at regular predetermined intervals.
62. The system of any of embodiments 40-61, wherein the one or more neural data structures comprises imaging data at an optical resolution.
63. The system of embodiment 62, wherein the optical resolution is at least 20 microns, 25 microns, 50 microns, 100 microns, 150 microns, 200 microns, 250 microns, 300 microns, or 500 microns.
64. The system of any of embodiments 40-63, wherein the one or more neural data structures comprises precursor imaging data at a computational superresolution.
65. The system of embodiment 40-64, wherein the computational superresolution is less than the optical resolution.
66. The system of any of embodiments 40-65, wherein the one or more neural data structures comprises imaging data at an imaging depth.
67. The system of embodiment 66, wherein the imaging depth is at least 0.04 cm, 0.1 cm, 0.2 cm, 0.4 cm, 0.5 cm, 2.0 cm, 4.0 cm, 6.0 cm, or 8.0 cm.
68. The system of any of embodiments 41-67, wherein the ultrasound waves comprise a frequency of at most 100 MHz, 60 MHz, 15 MHz, 7.5 MHz, 5.0 MHz, 2.5 MHz, or 1.0 MHz.
69. The system of embodiment 68, wherein the optical resolution corresponds to approximately the frequency.
70. The system of any of embodiments 68 or 69, wherein the computational superresolution corresponds to approximately the frequency.
71. The system of any of embodiments 40-70, wherein the one or more neural data structures comprises hemodynamic data.
72. The system of any of embodiments 40-71, wherein the one or more neural data structures comprises neural activity data.
73. The system of any of embodiments 40-72, wherein the one or more neural data structures comprises neural anatomy data.
74. The system of any of embodiments 40-73, wherein the ultrasound transducer is an implantable ultrasound transducer.
75. The system of any of embodiments 40-73, wherein the ultrasound transducer is a non-implantable ultrasound transducer.
76. The system of embodiment 75, wherein the non-implantable ultrasound transducer comprises a wearable ultrasound transducer.
77. The system of any of embodiments 40-76, wherein the sono-enhancing contrast agent comprises a biological sono-enhancing contrast agent, a polymeric sono-enhancing contrast agent, or a pharmaceutical sono-enhancing contrast agent.
78. The system of embodiment 77, wherein the biological sono-enhancing contrast agent comprises a genetically-encoded sono-enhancing contrast agent.
79. The system of embodiment 78, wherein the genetically-encoded sono-enhancing contrast agent comprises an ultrasonic reporter of calcium, an ultrasonic reporter of voltage, or an ultrasonic reporter of a neurotransmitter.
80. The system of any of embodiments 40-79, wherein the subject comprises a human subject or a non-human primate subject.
81. The system of any of embodiments 40-80, wherein the subject has, or is suspected of having, a neural dysfunction.
82. The system of embodiment 81, wherein the neural dysfunction comprises a motor dysfunction, a sensory dysfunction, a cognitive dysfunction, a neurological condition, or a neuropsychiatric dysfunction.
83. The system of embodiment 82, wherein the motor dysfunction comprises a spinal cord injury, amyotrophic lateral sclerosis, locked-in syndrome, stroke, Parkinson's disease, or essential tremor.
84. The system of embodiment 82 or 83, wherein the sensory dysfunction comprises blindness, deafness, epilepsy, a loss of chemosensation, or a loss of somatosensation.
85. The system of any of embodiments 82-84, wherein the neuropsychiatric dysfunction comprises clinical depression, schizophrenia, addiction, post-traumatic stress disorder, eating disorder, or sleep disorder.
86. The system of any of embodiments 40-85, wherein the ultrasound transducer provides a neural augmenting to the subject.
87. The system of any of embodiments 40-86, wherein the neural augmenting comprises sensorimotor integrating, sensory augmenting, integrating to a virtual reality, integrating to an augmented reality, modulating a brain state, or integrating to an artificial intelligence.
88. An ultrasound transducer configured to emit ultrasound waves, or receive the ultrasound waves, from a whole brain from a subject comprising a sono-enhancing contrast agent, the ultrasound transducer comprising:
   a sonolucent window;
   an ultrasonic transducer array, wherein
      an element in the ultrasonic transducer array is 100 to 500 microns wide, and
      the ultrasonic transducer array comprises at least 100 elements;
   a control circuit configured to receive or execute instructions for activating a modulating mode or a monitoring mode for the ultrasound transducer; and
   a processing circuit configured to transform the received ultrasound waves into one or more neural data structures.
89. An ultrasound transducer configured to emit ultrasound waves, or receive the ultrasound waves, from a whole brain from a subject comprising a sono-enhancing contrast agent, the ultrasound transducer comprising:
   a sonolucent window;
   an ultrasonic transducer array, wherein
      an element in the ultrasonic transducer array is 100 to 500 microns wide,
      the ultrasonic transducer array comprises at least 100 elements, and
      the ultrasonic transducer array is configured to receive or execute instructions for modulating the one or more portions of the nervous system from the subject; and
   a processing circuit configured to transform the received ultrasound waves into one or more neural data structures.
90. An ultrasound transducer configured to emit ultrasound waves, or receive the ultrasound waves, from a whole brain from a subject comprising a sono-enhancing contrast agent, the ultrasound transducer comprising:
   a sonolucent window;
   an ultrasonic transducer array, wherein
      an element in the ultrasonic transducer array is 100 to 500 microns wide,
      the ultrasonic transducer array comprises at least 100 elements, and
      the ultrasonic transducer array is configured to receive or execute instructions for monitoring the one or more portions of the nervous system from the subject; and
   a processing circuit configured to transform the received ultrasound waves into one or more neural data structures.
91. The ultrasound transducer of any of embodiments 88-90, wherein the one or more neural data structures corresponds to an imaging volume of at most 100 cm³, 700 cm³, or 1000 cm³.
92. The ultrasound transducer of any of embodiments 88-91, wherein the one or more neural data structures comprises imaging data at an optical resolution.
93. The ultrasound transducer of embodiment 92, wherein the optical resolution corresponds to approximately a width of the element in the ultrasonic transducer array.
94. The ultrasound transducer of embodiment 93, wherein the optical resolution corresponds to approximately the width of the element in the ultrasonic transducer array, based on an approximately one-to-one ratio.
95. The ultrasound transducer of embodiment 93 or 94, wherein the optical resolution is at least 100 microns, 150 microns, 250 microns, 300 microns or 500 microns.
96. The ultrasound transducer of any of embodiments 88-95, wherein the one or more neural data structures comprises precursor imaging data at a computational superresolution.
97. The ultrasound transducer of embodiment 96, wherein the computational superresolution corresponds to less than the width of the element in the ultrasonic transducer array.
98. The ultrasound transducer of embodiment 96 or 97, wherein the computational superresolution corresponds to approximately the width of the element in the ultrasonic transducer array, based on an approximately multi-to-one ratio.
99. The ultrasound transducer of any of embodiments 88-98, wherein the one or more neural data structures comprises imaging data at an imaging depth.
100. The ultrasound transducer of embodiment 99, wherein the imaging depth is at least 4.0 cm, 6.0 cm, or 8.0 cm.
101. The ultrasound transducer of embodiments 88-100, wherein the ultrasound waves comprise a frequency of at most 5.0 MHz, 2.5 MHz, or 1.0 MHz.
102. The ultrasound transducer of embodiment 101, wherein the optical resolution corresponds to approximately the frequency.
103. The ultrasound transducer of embodiment 100 or 101, wherein the computational superresolution corresponds to approximately the frequency.
104. An ultrasound transducer configured to emit ultrasound waves, or receive the ultrasound waves, from a brain region or a neural circuit from a subject comprising a sono-enhancing contrast agent, the ultrasound transducer comprising:
   a sonolucent window;
   an ultrasonic transducer array, wherein
      an element in the ultrasonic transducer array is 50 to 250 microns wide, and
      the ultrasonic transducer array comprises at least 100 elements;
   a control circuit configured to receive or execute instructions for activating a modulating mode or a monitoring mode for the ultrasound transducer; and
   a processing circuit configured to transform the received ultrasound waves into one or more neural data structures.
105. An ultrasound transducer configured to emit ultrasound waves, or receive the ultrasound waves, from a brain region or a neural circuit from a subject comprising a sono-enhancing contrast agent, the ultrasound transducer comprising:
   a sonolucent window;
   an ultrasonic transducer array, wherein
      an element in the ultrasonic transducer array is 50 to 250 microns wide,
      the ultrasonic transducer array comprises at least 100 elements, and
      the ultrasonic transducer array is configured to receive or execute instructions for modulating the one or more portions of the nervous system from the subject; and
   a processing circuit configured to transform the received ultrasound waves into one or more neural data structures.
106. An ultrasound transducer configured to emit ultrasound waves, or receive the ultrasound waves, from a brain region or a neural circuit from a subject comprising a sono-enhancing contrast agent, the ultrasound transducer comprising:
   a sonolucent window;
   an ultrasonic transducer array, wherein
      an element in the ultrasonic transducer array is 50 to 250 microns wide,
      the ultrasonic transducer array comprises at least 100 elements, and
      the ultrasonic transducer array is configured to receive or execute instructions for monitoring the one or more portions of the nervous system from the subject; and
   a processing circuit configured to transform the received ultrasound waves into one or more neural data structures.
107. The ultrasound transducer of any of embodiments 104-106, wherein the one or more neural data structures corresponds to an imaging volume of at most 0.6 cm³, 12 cm³, or 100 cm³.
108. The ultrasound transducer of any of embodiments 104-107, wherein the one or more neural data structures comprises imaging data at an optical resolution.
109. The ultrasound transducer of embodiment 108, wherein the optical resolution corresponds to approximately a width of the element in the ultrasonic transducer array.
110. The ultrasound transducer of embodiment 109, wherein the optical resolution corresponds to approximately the width of the element in the ultrasonic transducer array, based on an approximately one-to-one ratio.
111. The ultrasound transducer of embodiment 109 or 110, wherein the optical resolution is at least 50 microns, 100 microns, 150 microns, 200 microns, or 250 microns.
112. The ultrasound transducer of any of embodiments 104-111, wherein the one or more neural data structures comprises precursor imaging data at a computational superresolution.
113. The ultrasound transducer of embodiment 112, wherein the computational superresolution corresponds to less than the width of the element in the ultrasonic transducer array.
114. The ultrasound transducer of embodiment 112 or 113, wherein the computational superresolution corresponds to approximately the width of the element in the ultrasonic transducer array, based on an approximately multi-to-one ratio.
115. The ultrasound transducer of any of embodiments 104-114, wherein the one or more neural data structures comprises imaging data at an imaging depth.
116. The ultrasound transducer of embodiment 115, wherein the imaging depth is at least 6.0 cm, or 8.0 cm.
117. The ultrasound transducer of embodiments 104-116, wherein the ultrasound waves comprise a frequency of at most 15 MHz, 7.5 MHz, or 5.0 MHz.
118. The ultrasound transducer of embodiment 117, wherein the optical resolution corresponds to approximately the frequency.
119. The ultrasound transducer of embodiment 117 or 118, wherein the computational superresolution corresponds to approximately the frequency.
120. An ultrasound transducer configured to emit ultrasound waves, or receive the ultrasound waves, from a neural circuit or a single neuron from a subject comprising a sono-enhancing contrast agent, the ultrasound transducer comprising:
   a sonolucent window;
   an ultrasonic transducer array, wherein
      an element in the ultrasonic transducer array is 20 to 50 microns wide, and
      the ultrasonic transducer array comprises at least 100 elements;
   a control circuit configured to receive or execute instructions for activating a modulating mode or a monitoring mode for the ultrasound transducer; and
   a processing circuit configured to transform the received ultrasound waves into one or more neural data structures.
121. An ultrasound transducer configured to emit ultrasound waves, or receive the ultrasound waves, from a neural circuit or a single neuron from a subject comprising a sono-enhancing contrast agent, the ultrasound transducer comprising:
   a sonolucent window;
   an ultrasonic transducer array, wherein
      an element in the ultrasonic transducer array is 20 to 50 microns wide,
      the ultrasonic transducer array comprises at least 100 elements, and
      the ultrasonic transducer array is configured to receive or execute instructions for modulating the one or more portions of the nervous system from the subject; and
   a processing circuit configured to transform the received ultrasound waves into one or more neural data structures.
122. An ultrasound transducer configured to emit ultrasound waves, or receive the ultrasound waves, from a neural circuit or a single neuron from a subject comprising a sono-enhancing contrast agent, the ultrasound transducer comprising:
   a sonolucent window;
   an ultrasonic transducer array, wherein
      an element in the transducer array is 20 to 50 microns wide,
      the ultrasonic transducer array comprises at least 100 elements, and
      the ultrasonic transducer array is configured to receive or execute instructions for monitoring the one or more portions of the nervous system from the subject; and
   a processing circuit configured to transform the received ultrasound waves into one or more neural data structures.
123. The ultrasound transducer of any of embodiments 120-122, wherein the one or more neural data structures corresponds to an imaging volume of at most 0.2 cm³ or 0.6 cm³.
124. The ultrasound transducer of any of embodiments 120-123, wherein the one or more neural data structures comprises imaging data at an optical resolution.
125. The ultrasound transducer of embodiment 124, wherein the optical resolution corresponds to approximately a width of the element in the ultrasonic transducer array.
126. The ultrasound transducer of embodiment 125, wherein the optical resolution corresponds to approximately the width of the element in the ultrasonic transducer array, based on an approximately one-to-one ratio.
127. The ultrasound transducer of embodiment 125 or 126, wherein the optical resolution is at most 50 microns, 25 microns, or 20 microns.
128. The ultrasound transducer of any of embodiments 120-127, wherein the one or more neural data structures comprises precursor imaging data at a computational superresolution.
129. The ultrasound transducer of embodiment 128, wherein the computational superresolution corresponds to less than the width of the element in the ultrasonic transducer array.
130. The ultrasound transducer of embodiment 128 or 129, wherein the computational superresolution corresponds to approximately the width of the element in the ultrasonic transducer array, based on an approximately multi-to-one ratio.
131. The ultrasound transducer of any of embodiments 120-130, wherein the one or more neural data structures comprises imaging data at an imaging depth.
132. The ultrasound transducer of embodiment 131, wherein the imaging depth is at most 0.2 cm, 0.5 cm, or 2.0 cm.
133. The ultrasound transducer of embodiments 120-132, wherein the ultrasound waves comprise a frequency of at least 15 MHz, 60 MHz, or 100 MHz
134. The ultrasound transducer of embodiment 133, wherein the optical resolution corresponds to approximately the frequency.
135. The ultrasound transducer of embodiment 133 or 134, wherein the computational superresolution corresponds to approximately the frequency.
136. The ultrasound transducer of any of embodiments 88-135, wherein the one or more neural data structures comprises hemodynamic data.
137. The ultrasound transducer of any of embodiments 88-136, wherein the one or more neural data structures comprises neural activity data.
138. The ultrasound transducer of any of embodiments 88-187, wherein the one or more neural data structures comprises neural anatomy data.
139. The ultrasound transducer of any of embodiments 88-138, wherein the element in the ultrasonic transducer array is circular, elliptical, square, rectangular, hexagonal, regular, or irregular, in shape.
140. The ultrasound transducer of any of embodiments 88-139, wherein the ultrasonic transducer array is a capacitive micromachined ultrasonic transducer (CMUT) array or a piezoelectric micromachined ultrasonic transducer (PMUT) array.
141. The ultrasound transducer of embodiment 140, wherein the CMUT array comprises a flat membrane CMUT, a curved membrane CMUT, a dual-electrode CMUT, a collapsed-mode CMUT, a wafer-bonded CMUT, a surface-micromachined CMUT, a CMUT-on-CMOS integrated device, a piston-shaped CMUT, an annular array CMUT, or a flexible CMUT.
142. The ultrasound transducer of embodiment 141, wherein the PMUT array comprises a unimorph PMUT, a bimorph PMUT, a dome-shaped PMUT, a suspended membrane PMUT, an aluminum nitride PMUT, a lead zirconate titanate PMUT, a scandium-doped aluminum nitride PMUT, a lithium niobate PMUT, a polyvinylidene fluoride PMUT, a thin-film PMUT, or a flexible PMUT.
143. The ultrasound transducer of any of embodiments 88-142, wherein the ultrasound transducer is an implantable ultrasound transducer.
144. The ultrasound transducer of any of embodiments 88-142, wherein the ultrasound transducer is a non-implantable ultrasound transducer.
145. The ultrasound transducer of embodiment 144, wherein the non-implantable ultrasound transducer comprises a wearable ultrasound transducer.
146. The ultrasound transducer of any of embodiments 88-145, wherein the sono-enhancing contrast agent comprises a biological sono-enhancing contrast agent, a polymeric sono-enhancing contrast agent, or a pharmaceutical sono-enhancing contrast agent.
147. The ultrasound transducer of embodiment 146, wherein the biological sono-enhancing contrast agent comprises a genetically-encoded sono-enhancing contrast agent.
148. The ultrasound transducer of embodiment 147, wherein the genetically-encoded sono-enhancing contrast agent comprises an ultrasonic reporter of calcium, an ultrasonic reporter of voltage, or an ultrasonic reporter of a neurotransmitter.
149. The ultrasound transducer of any of embodiments 88-148, wherein the subject comprises a human subject or a non-human primate subject.
150. The ultrasound transducer of any of embodiments 88-149, wherein the subject has, or is suspected of having, a neural dysfunction.
151. The ultrasound transducer of embodiment 150, wherein the neural dysfunction comprises a motor dysfunction, a sensory dysfunction, a cognitive dysfunction, a neurological condition, or a neuropsychiatric dysfunction.
152. The ultrasound transducer of embodiment 151, wherein the motor dysfunction comprises a spinal cord injury, amyotrophic lateral sclerosis, locked-in syndrome, stroke, Parkinson's disease, or essential tremor.
153. The ultrasound transducer of embodiment 151 or 152, wherein the sensory dysfunction comprises blindness, deafness, epilepsy, a loss of chemosensation, or a loss of somatosensation.
154. The ultrasound transducer of any of embodiments 151-153, wherein the neuropsychiatric dysfunction comprises, clinical depression, schizophrenia, addiction, post-traumatic stress disorder, eating disorder, or sleep disorder.
155. The ultrasound transducer of any of embodiments 88-154, wherein the ultrasound transducer provides a neural augmenting to the subject.
156. The ultrasound transducer of any of embodiments 88-155, wherein the neural augmenting comprises sensorimotor integrating, sensory augmenting, integrating to a virtual reality, integrating to an augmented reality, modulating a brain state, or integrating to an artificial intelligence.
157. A method of modulating or monitoring a whole brain of a subject comprising a sono-enhancing contrast agent, comprising:
   emitting ultrasound waves to the whole brain, from an ultrasound transducer corresponding to the ultrasound transducer of any of embodiments 1-156;
   sono-enhancing the emitted ultrasound waves, via the sono-enhancing contrast agent in the subject, to form sono-enhanced ultrasound waves;
   receiving the sono-enhanced ultrasound waves from the subject, by the ultrasound transducer; and
   determining one or more neural data structures, using one or more processors, based on the received sono-enhanced ultrasound waves.
158. The method of embodiment 157, wherein the ultrasound transducer comprises an ultrasonic transducer array, wherein an element in the ultrasonic transducer array is 100 to 500 microns wide.
159. The method of embodiment 157 or 158, wherein the one or more neural data structures corresponds to an imaging volume of at most 100 cm³, 700 cm³, or 1000 cm³.
160. The method of any of embodiments 157-159, wherein the one or more neural data structures comprises imaging data at an optical resolution, wherein the optical resolution is at least 100 microns, 150 microns, 250 microns, 300 microns or 500 microns.
161. The method of any of embodiments 157-160, wherein the one or more neural data structures comprises imaging data at an imaging depth, wherein the imaging depth is at least 4.0 cm, 6.0 cm, or 8.0 cm.
162. The method of any of embodiments 157-161, wherein the ultrasound waves comprise a frequency of at most 5.0 MHz, 2.5 MHz, or 1.0 MHz.
163. A method of modulating or monitoring a brain region or a neural circuit of a subject comprising a sono-enhancing contrast agent, comprising:
   emitting ultrasound waves to the brain region or the neural circuit, from an ultrasound transducer corresponding to the ultrasound transducer of any of embodiments 1-156;
   sono-enhancing the emitted ultrasound waves, via the sono-enhancing contrast agent in the subject, to form sono-enhanced ultrasound waves;
   receiving the sono-enhanced ultrasound waves from the subject, by the ultrasound transducer; and
   determining one or more neural data structures, using one or more processors, based on the received sono-enhanced ultrasound waves.
164. The method of embodiment 163, wherein the ultrasound transducer comprises an ultrasonic transducer array, wherein an element in the ultrasonic transducer array is 50 to 250 microns wide.
165. The method of embodiment 163 or 164, wherein the one or more neural data structures corresponds to an imaging volume of at most 0.6 cm³, 12 cm³, or 100 cm³.
166. The method of any of embodiments 163-165, wherein the one or more neural data structures comprises imaging data at an optical resolution, wherein the optical resolution is at least 50 microns, 100 microns, 150 microns, 200 microns, or 250 microns.
167. The method of any of embodiments 163-166, wherein the one or more neural data structures comprises imaging data at an imaging depth, wherein the imaging depth is at least 2.0 cm, or 4.0 cm.
168. The method of any of embodiments 163-167, wherein the ultrasound waves comprise a frequency of at most 15 MHz, 7.5 MHz, or 5.0 MHz.
169. A method of modulating or monitoring a neural circuit or a single neuron of a subject comprising a sono-enhancing contrast agent, comprising:
   emitting ultrasound waves to the neural circuit or the single neuron, from an ultrasound transducer corresponding to the ultrasound transducer of any of embodiments 1-156;
   sono-enhancing the emitted ultrasound waves, via the sono-enhancing contrast agent in the subject, to form sono-enhanced ultrasound waves;
   receiving the sono-enhanced ultrasound waves from the subject, by the ultrasound transducer; and
   determining one or more neural data structures, using one or more processors, based on the received sono-enhanced ultrasound waves.
170. The method of embodiment 169, wherein the ultrasound transducer comprises an ultrasonic transducer array, wherein an element in the ultrasonic transducer array is 20 to 50 microns wide.
171. The method of embodiment 169 or 170, wherein the one or more neural data structures corresponds to an imaging volume of at most 0.2 cm³, or 0.6 cm³.
172. The method of any of embodiments 169-171, wherein the one or more neural data structures comprises imaging data at an optical resolution, wherein the optical resolution is at least 20 microns, 25 microns, or 50 microns.
173. The method of any of embodiments 169-172, wherein the one or more neural data structures comprises imaging data at an imaging depth, wherein the imaging depth is at least 0.04 cm, 0.1 cm, or 0.4 cm.
174. The method of any of embodiments 169-173, wherein the ultrasound waves comprise a frequency of at most 100 MHz, 60 MHz, or 15 MHz.
175. The method of any of embodiments 157-174, wherein the one or more neural data structures comprises precursor imaging data at a computational superresolution.
176. The method of embodiment 175, wherein the computational superresolution corresponds to less than a width of the element in the ultrasonic transducer array.
177. The method of any of embodiments 157-176, wherein the one or more neural data structures comprises hemodynamic data.
178. The method of any of embodiments 157-177, wherein the one or more neural data structures comprises neural activity data.
179. The method of any of embodiments 157-178, wherein the one or more neural data structures comprises neural anatomy data.
180. The method of any of embodiments 157-179, wherein the element in the ultrasonic transducer array is circular, elliptical, square, rectangular, hexagonal, regular, or irregular, in shape.
181. The method of any of embodiments 157-180, wherein the ultrasonic transducer array is a capacitive micromachined ultrasonic transducer (CMUT) array or a piezoelectric micromachined ultrasonic transducer (PMUT) array.
182. The method of embodiment 181, wherein the CMUT array comprises a flat membrane CMUT, a curved membrane CMUT, a dual-electrode CMUT, a collapsed-mode CMUT, a wafer-bonded CMUT, a surface-micromachined CMUT, a CMUT-on-CMOS integrated device, a piston-shaped CMUT, an annular array CMUT, or a flexible CMUT.
183. The method of embodiment 181, wherein the PMUT array comprises a unimorph PMUT, a bimorph PMUT, a dome-shaped PMUT, a suspended membrane PMUT, an aluminum nitride PMUT, a lead zirconate titanate PMUT, a scandium-doped aluminum nitride PMUT, a lithium niobate PMUT, a polyvinylidene fluoride PMUT, a thin-film PMUT, or a flexible PMUT.
184. The method of any of embodiments 157-183, wherein the ultrasound transducer is an implantable ultrasound transducer.
185. The method of any of embodiments 157-183, wherein the ultrasound transducer is a non-implantable ultrasound transducer.
186. The method of embodiment 185, wherein the non-implantable ultrasound transducer comprises a wearable ultrasound transducer.
187. The method of any of embodiments 157-186, wherein the sono-enhancing contrast agent comprises a biological sono-enhancing contrast agent, a polymeric sono-enhancing contrast agent, or a pharmaceutical sono-enhancing contrast agent.
188. The method of embodiment 157-187, wherein the biological sono-enhancing contrast agent comprises a genetically-encoded sono-enhancing contrast agent.
189. The method of embodiment 157-188, wherein the genetically-encoded sono-enhancing contrast agent comprises an ultrasonic reporter of calcium, an ultrasonic reporter of voltage, or an ultrasonic reporter of a neurotransmitter.
190. The method of any of embodiments 157-189, wherein the subject comprises a human subject or a non-human primate subject.
191. The method of any of embodiments 157-190, wherein the subject has, or is suspected of having, a neural dysfunction.
192. The method of embodiment 191, wherein the neural dysfunction comprises a motor dysfunction, a sensory dysfunction, a cognitive dysfunction, a neurological condition, or a neuropsychiatric dysfunction.
193. The method of embodiment 192, wherein the motor dysfunction comprises a spinal cord injury, amyotrophic lateral sclerosis, locked-in syndrome, stroke, Parkinson's disease, or essential tremor.
194. The method of embodiment 192 or 193, wherein the sensory dysfunction comprises blindness, deafness, epilepsy, a loss of chemosensation, or a loss of somatosensation.
195. The method of any of embodiments 192-194, wherein the neuropsychiatric dysfunction comprises clinical depression, schizophrenia, addiction, post-traumatic stress disorder, eating disorder, or sleep disorder.
196. The method of any of embodiments 192-195, wherein the ultrasound transducer provides a neural augmenting to the subject.
197. The method of any of embodiments 192-196, wherein the neural augmenting comprises sensorimotor integrating, sensory augmenting, integrating to a virtual reality, integrating to an augmented reality, modulating a brain state, or integrating to an artificial intelligence.
198. A system comprising:
   one or more processors; and
   a memory communicatively coupled to the one or more processors and configured to store instructions that, when executed by the one or more processors, cause the system to:
      emit ultrasound waves to a whole brain of a subject, from an ultrasound transducer corresponding to the ultrasound transducer of any of embodiments 1-156;
      sono-enhance the emitted ultrasound waves, via the sono-enhancing contrast agent in the subject, to form sono-enhanced ultrasound waves;
      receive the sono-enhanced ultrasound waves from the subject, by the ultrasound transducer; and
      determine one or more neural data structures, using one or more processors, based on the received sono-enhanced ultrasound waves.
199. The system of embodiment 198, wherein the ultrasound transducer comprises an ultrasonic transducer array, wherein an element in the ultrasonic transducer array is 100 to 500 microns wide.
200. The system of embodiment 198 or 199, wherein the one or more neural data structures corresponds to an imaging volume of at most 100 cm³, 700 cm³, or 1000 cm³.
201. The system of any of embodiments 198-200, wherein the one or more neural data structures comprises imaging data at an optical resolution, wherein the optical resolution is at least 100 microns, 150 microns, 250 microns, 300 microns or 500 microns.
202. The system of any of embodiments 198-201, wherein the one or more neural data structures comprises imaging data at an imaging depth, wherein the imaging depth is at least 4.0 cm, 6.0 cm, or 8.0 cm.
203. The system of any of embodiments 198-202, wherein the ultrasound waves comprise a frequency of at most 5.0 MHz, 2.5 MHz, or 1.0 MHz.
204. A system comprising:
   one or more processors; and
   a memory communicatively coupled to the one or more processors and configured to store instructions that, when executed by the one or more processors, cause the system to:
      emit ultrasound waves to a brain region or a neural circuit of a subject, from an ultrasound transducer corresponding to the ultrasound transducer of any of embodiments 1-156;
      sono-enhance the emitted ultrasound waves, via the sono-enhancing contrast agent in the subject, to form sono-enhanced ultrasound waves;
      receive the sono-enhanced ultrasound waves from the subject, by the ultrasound transducer; and
      determine one or more neural data structures, using one or more processors, based on the received sono-enhanced ultrasound waves.
205. The system of embodiment 204, wherein the ultrasound transducer comprises an ultrasonic transducer array, wherein an element in the ultrasonic transducer array is 50 to 250 microns wide.
206. The system of embodiment 204 or 205, wherein the one or more neural data structures corresponds to an imaging volume of at most 0.6 cm³, 12 cm³, or 100 cm³.
207. The system of any of embodiments 204-206, wherein the one or more neural data structures comprises imaging data at an optical resolution, wherein the optical resolution is at least 50 microns, 100 microns, 150 microns, 200 microns, or 250 microns.
208. The system of any of embodiments 204-207, wherein the one or more neural data structures comprises imaging data at an imaging depth, wherein the imaging depth is at least 2.0 cm, or 4.0 cm.
209. The system of any of embodiments 204-208, wherein the ultrasound waves comprise a frequency of at most 15 MHz, 7.5 MHz, or 5.0 MHz.
210. A system comprising:
   one or more processors; and
   a memory communicatively coupled to the one or more processors and configured to store instructions that, when executed by the one or more processors, cause the system to:
      emit ultrasound waves to a neural circuit or a single neuron of a subject, from an ultrasound transducer corresponding to the ultrasound transducer of any of embodiments 1-156;
      sono-enhance the emitted ultrasound waves, via the sono-enhancing contrast agent in the subject, to form sono-enhanced ultrasound waves;
      receive the sono-enhanced ultrasound waves from the subject, by the ultrasound transducer; and
      determine one or more neural data structures, using one or more processors, based on the received sono-enhanced ultrasound waves.
211. The system of embodiment 210, wherein the ultrasound transducer comprises an ultrasonic transducer array, wherein an element in the ultrasonic transducer array is 20 to 50 microns wide.
212. The system of embodiment 210 or 211, wherein the one or more neural data structures corresponds to an imaging volume of at most 0.2 cm³, or 0.6 cm³.
213. The system of any of embodiments 210-212, wherein the one or more neural data structures comprises imaging data at an optical resolution, wherein the optical resolution is at least 20 microns, 25 microns, or 50 microns.
214. The system of any of embodiments 210-213, wherein the one or more neural data structures comprises imaging data at an imaging depth, wherein the imaging depth is at least 0.04 cm, 0.1 cm, or 0.4 cm.
215. The system of any of embodiments 210-214, wherein the ultrasound waves comprise a frequency of at most 100 MHz, 60 MHz, or 15 MHz.
216. A non-transitory computer-readable storage medium storing one or more programs, the one or more programs comprising instructions, which when executed by one or more processors of a system, cause the system to:
   emit ultrasound waves to a whole brain of a subject, from an ultrasound transducer corresponding to the ultrasound transducer of any of embodiments 1-156;
   sono-enhance the emitted ultrasound waves, via the sono-enhancing contrast agent in the subject, to form sono-enhanced ultrasound waves;
   receive the sono-enhanced ultrasound waves from the subject, by the ultrasound transducer; and
   determine one or more neural data structures, using one or more processors, based on the received sono-enhanced ultrasound waves.
217. The non-transitory computer-readable storage medium of embodiment 216, wherein the ultrasound transducer comprises an ultrasonic transducer array, wherein an element in the ultrasonic transducer array is 20 to 50 microns wide.
218. The non-transitory computer-readable storage medium of embodiment 216 or 217, wherein the one or more neural data structures corresponds to an imaging volume of at most 0.2 cm³, or 0.6 cm³.
219. The non-transitory computer-readable storage medium of any of embodiments 216-218, wherein the one or more neural data structures comprises imaging data at an optical resolution, wherein the optical resolution is at least 20 microns, 25 microns, or 50 microns.
220. The non-transitory computer-readable storage medium of any of embodiments 216-219, wherein the one or more neural data structures comprises imaging data at an imaging depth, wherein the imaging depth is at least 0.04 cm, 0.1 cm, or 0.4 cm.
221. The non-transitory computer-readable storage medium of any of embodiments 216-220, wherein the ultrasound waves comprise a frequency of at most 100 MHz, 60 MHz, or 15 MHz.
222. A non-transitory computer-readable storage medium storing one or more programs, the one or more programs comprising instructions, which when executed by one or more processors of a system, cause the system to:
   emit ultrasound waves to a brain region or a neural circuit of a subject, from an ultrasound transducer corresponding to the ultrasound transducer of any of embodiments 1-156;
   sono-enhance the emitted ultrasound waves, via the sono-enhancing contrast agent in the subject, to form sono-enhanced ultrasound waves;
   receive the sono-enhanced ultrasound waves from the subject, by the ultrasound transducer; and
   determine one or more neural data structures, using one or more processors, based on the received sono-enhanced ultrasound waves.
223. The non-transitory computer-readable storage medium of embodiment 222, wherein the ultrasound transducer comprises an ultrasonic transducer array, wherein an element in the ultrasonic transducer array is 50 to 250 microns wide.
224. The non-transitory computer-readable storage medium of embodiment 222 or 223, wherein the one or more neural data structures corresponds to an imaging volume of at most 0.6 cm³, 12 cm³, or 100 cm³.
225. The non-transitory computer-readable storage medium of any of embodiments 222-224, wherein the one or more neural data structures comprises imaging data at an optical resolution, wherein the optical resolution is at least 50 microns, 100 microns, 150 microns, 200 microns, or 250 microns.
226. The non-transitory computer-readable storage medium of any of embodiments 222-225, wherein the one or more neural data structures comprises imaging data at an imaging depth, wherein the imaging depth is at least 2.0 cm, or 4.0 cm.
227. The non-transitory computer-readable storage medium of any of embodiments 222-226, wherein the ultrasound waves comprise a frequency of at most 15 MHz, 7.5 MHz, or 5.0 MHz.
228. A non-transitory computer-readable storage medium storing one or more programs, the one or more programs comprising instructions, which when executed by one or more processors of a system, cause the system to:
   emit ultrasound waves to a neural circuit or a single neuron of a subject, from an ultrasound transducer corresponding to the ultrasound transducer of any of embodiments 1-156;
   sono-enhance the emitted ultrasound waves, via the sono-enhancing contrast agent in the subject, to form sono-enhanced ultrasound waves;
   receive the sono-enhanced ultrasound waves from the subject, by the ultrasound transducer; and
   determine one or more neural data structures, using one or more processors, based on the received sono-enhanced ultrasound waves.
229. The non-transitory computer-readable storage medium of embodiment 228, wherein the ultrasound transducer comprises an ultrasonic transducer array, wherein an element in the ultrasonic transducer array is 20 to 50 microns wide.
230. The non-transitory computer-readable storage medium of embodiment 228 or 229, wherein the one or more neural data structures corresponds to an imaging volume of at most 0.2 cm³, or 0.6 cm³.
231. The non-transitory computer-readable storage medium of any of embodiments 228-230, wherein the one or more neural data structures comprises imaging data at an optical resolution, wherein the optical resolution is at least 20 microns, 25 microns, or 50 microns.
232. The non-transitory computer-readable storage medium of any of embodiments 228-231, wherein the one or more neural data structures comprises imaging data at an imaging depth, wherein the imaging depth is at least 0.04 cm, 0.1 cm, or 0.4 cm.
233. The non-transitory computer-readable storage medium of any of embodiments 228-232, wherein the ultrasound waves comprise a frequency of at most 100 MHz, 60 MHz, or 15 MHz.
234. A method for modulating a first ongoing psychophysical state of a subject, based on neural activity from one or more portions of a nervous system of a subject comprising a sono-enhancing contrast agent, comprising:
   emitting first ultrasound waves to the subject, from an ultrasound transducer corresponding to the ultrasound transducer of any of embodiments 1-156;
   receiving first sono-enhanced ultrasound waves from the subject, wherein the first sono-enhanced ultrasound waves comprise the first emitted ultrasound waves enhanced via the sono-enhancing contrast agent;
   determining a first plurality of one or more neural data structures, using one or more processors, based on the first received sono-enhanced ultrasound waves;
   decoding the first plurality into a first high-dimensional goal manifold, via a predictive machine learning algorithm;
   determining first instructions based on the first high-dimensional goal manifold, via an instruction-determining machine learning algorithm; and
   modulating the first ongoing psychophysical state of the subject, based on the first instructions.
235. The method of embodiment 234, further comprising:
   emitting second ultrasound waves to the subject, from the ultrasound transducer;
   receiving second sono-enhanced ultrasound waves from the subject, wherein the second sono-enhanced ultrasound waves comprise the second emitted ultrasound waves enhanced via the sono-enhancing contrast agent;
   determining a second plurality of one or more neural data structures, using one or more processors, based on the second received sono-enhanced ultrasound waves;
   decoding the second plurality or the first plurality into a second high-dimensional goal manifold, via a predictive machine learning algorithm;
   determining second instructions based on the first high-dimensional goal manifold, via an instruction-determining machine learning algorithm; and
   modulating the second ongoing psychophysical state of the subject, based on the second instructions.
236. A method for modulating a first ongoing psychophysical state of a subject, based on neural activity from one or more portions of a nervous system of a subject comprising a sono-enhancing contrast agent, comprising:
   emitting first ultrasound waves to the subject, from an ultrasound transducer corresponding to the ultrasound transducer of any of embodiments 1-156, to the one or more portions of the nervous system;
   receiving first sono-enhanced ultrasound waves from the subject, wherein the first sono-enhanced ultrasound waves comprise the first emitted ultrasound waves enhanced via the sono-enhancing contrast agent;
   determining a first plurality of one or more neural data structures, using one or more processors, based on the first received sono-enhanced ultrasound waves;
   decoding the first plurality, via an instruction-determining machine learning algorithm, into first instructions; and
   modulating the first ongoing psychophysical state of the subject, based on the first instructions.
237. The method of embodiment 236, further comprising:
   emitting second ultrasound waves to the subject, from an ultrasound transducer corresponding to the ultrasound transducer of any of embodiments 1-156, to the one or more portions of the nervous system;
   receiving second sono-enhanced ultrasound waves from the subject, wherein the second sono-enhanced ultrasound waves comprise the second emitted ultrasound waves enhanced via the sono-enhancing contrast agent;
   determining a second plurality of one or more neural data structures, using one or more processors, based on the second received sono-enhanced ultrasound waves;
   decoding the second plurality, via an instruction-determining machine learning algorithm, into second instructions; and
   modulating the second ongoing psychophysical state of the subject, based on the second instructions.
238. The method of any of embodiments 235-237, wherein the first high-dimensional goal manifold or the second high-dimensional goal manifold comprises dimensions representing, at least in part, predictions across multiple timescales, environmental variables, or personal values of the subject.
239. The method of embodiment 238, wherein the multiple timescales comprise milliseconds-long timescales, seconds-long timescales, minutes-long timescales, hours-long timescales. days-long timescales, weeks-long timescales, months-long timescales, or years-long timescales.
240. The method of any of embodiments 235-239, wherein the modulating the first ongoing psychophysical state or the modulating the second ongoing psychophysical state comprises modulating the neural activity of the subject.
241. The method of embodiment 240, wherein the modulating the neural activity of the subject comprises the emitting of the ultrasound waves to the subject, from the ultrasound transducer.
242. The method of embodiment 241, wherein the modulating the neural activity of the subject comprises electro-stimulating the one or more portions of the nervous system of the subject via one or more electrodes.
243. The method of embodiment 242, wherein the electro-stimulating comprises deep brain stimulation, transcranial magnetic stimulation (TMS), repetitive TMS, vagus nerve stimulation, transcranial direct current stimulation, or electrocorticography.
244. The method of any of embodiments 234-243, wherein the determining the first plurality occurs before the determining the second plurality.
245. The method of any of embodiments 234-244, wherein the one or more portions of the nervous system from the subject comprises a whole brain, a brain region, a neural circuit, or a single neuron.
246. The method of any of embodiments 234-245, wherein the one or more portions of the nervous system from the subject comprise a dorsolateral prefrontal cortex, frontopolar cortex, anterior cingulate cortex, inferotemporal cortex, visual cortex, auditory cortex, somatosensory cortex, posterior parietal cortex, temporoparietal junction, nucleus accumbens, ventral tegmental area, basal ganglia, orbitofrontal cortex, amygdala, insula, ventromedial prefrontal cortex, hippocampus, entorhinal cortex, parahippcampal gyrus, locus coerulus, reticular formation, ventral striatum, anterior insula, Broca's area, Wernicke's area, medial prefrontal cortex, temporoparietal junction, anterior insula, posterior insula, supplementary motor area, pre-supplementary motor area, or a combination thereof.
247. The method of any of embodiments 234-246, wherein the predictive machine learning algorithm or the instruction-determining machine learning algorithm is trained on ultrasound imaging data, radiofrequency data, electrophysiology data, functional magnetic imaging (fMRI) data, structural magnetic resonance imaging scan data, diffusion tensor imaging data, computed tomography scan data, or behavioral data.
248. The method of any of embodiments 234-247, wherein the predictive machine learning algorithm or the instruction-determining machine learning algorithm comprises an artificial neural network, a deep neural network, a convolutional neural network, a generative adversarial network, a deep unfolding network, a tokenizer, a transformer, an encoder, a decoder, an autoencoder, a Markov chain, a hidden Markov model, Bayesian optimization, a dimensionality reduction technique, a generalized linear model, a multi-layer perceptron, or a support vector machine.
249. The method of any of embodiments 234-248, wherein the one or more neural data structures comprises precursor imaging data at a computational superresolution.
250. The method of embodiment 249, wherein the computational superresolution corresponds to less than a width of the element in the ultrasonic transducer array.
251. The method of any of embodiments 234-250, wherein the one or more neural data structures comprises hemodynamic data.
252. The method of any of embodiments 234-251, wherein the one or more neural data structures comprises neural activity data.
253. The method of any of embodiments 234-252, wherein the one or more neural data structures comprises neural anatomy data.
254. The method of any of embodiments 234-253, wherein the element in the ultrasonic transducer array is circular, elliptical, square, rectangular, hexagonal, regular, or irregular, in shape.
255. The method of any of embodiments 234-254, wherein the ultrasonic transducer array is a capacitive micromachined ultrasonic transducer (CMUT) array or a piezoelectric micromachined ultrasonic transducer (PMUT) array.
256. The method of embodiment 255, wherein the CMUT array comprises a flat membrane CMUT, a curved membrane CMUT, a dual-electrode CMUT, a collapsed-mode CMUT, a wafer-bonded CMUT, a surface-micromachined CMUT, a CMUT-on-CMOS integrated device, a piston-shaped CMUT, an annular array CMUT, or a flexible CMUT.
257. The method of embodiment 256, wherein the PMUT array comprises a unimorph PMUT, a bimorph PMUT, a dome-shaped PMUT, a suspended membrane PMUT, an aluminum nitride PMUT, a lead zirconate titanate PMUT, a scandium-doped aluminum nitride PMUT, a lithium niobate PMUT, a polyvinylidene fluoride PMUT, a thin-film PMUT, or a flexible PMUT.
258. The method of any of embodiments 234-257, wherein the ultrasound transducer is an implantable ultrasound transducer.
259. The method of any of embodiments 234-258, wherein the ultrasound transducer is a non-implantable ultrasound transducer.
260. The method of embodiment 259, wherein the non-implantable ultrasound transducer comprises a wearable ultrasound transducer.
261. The method of any of embodiments 234-260, wherein the sono-enhancing contrast agent comprises a biological sono-enhancing contrast agent, a polymeric sono-enhancing contrast agent, or a pharmaceutical sono-enhancing contrast agent.
262. The method of embodiment 261, wherein the biological sono-enhancing contrast agent comprises a genetically-encoded sono-enhancing contrast agent.
263. The method of embodiment 262, wherein the genetically-encoded sono-enhancing contrast agent comprises an ultrasonic reporter of calcium, an ultrasonic reporter of voltage, or an ultrasonic reporter of a neurotransmitter.
264. The method of any of embodiments 234-263, wherein the subject comprises a human subject or a non-human primate subject.
265. The method of any of embodiments 234-264, wherein the subject has, or is suspected of having, a neural dysfunction.
266. The method of embodiment 265, wherein the neural dysfunction comprises a motor dysfunction, a sensory dysfunction, a cognitive dysfunction, a neurological condition, or a neuropsychiatric dysfunction.
267. The method of embodiment 266, wherein the motor dysfunction comprises a spinal cord injury, amyotrophic lateral sclerosis, locked-in syndrome, stroke, Parkinson's disease, or essential tremor.
268. The method of embodiment 266 or 267 wherein the sensory dysfunction comprises blindness, deafness, epilepsy, a loss of chemosensation, or a loss of somatosensation.
269. The method of any of embodiments 266-268, wherein the neuropsychiatric dysfunction comprises clinical depression, schizophrenia, addiction, post-traumatic stress disorder, eating disorder, or sleep disorder.
270. The method of any of embodiments 234-269, wherein the ultrasound transducer provides a neural augmenting to the subject.
271. The method of any of embodiments 234-270, wherein the neural augmenting comprises sensorimotor integrating, sensory augmenting, integrating to a virtual reality, integrating to an augmented reality, modulating a brain state, or integrating to an artificial intelligence.
272. A system comprising:
   one or more processors; and
   a memory communicatively coupled to the one or more processors and configured to store instructions that, when executed by the one or more processors, cause the system to:
      emit first ultrasound waves to the subject, from an ultrasound transducer corresponding to the ultrasound transducer of any of embodiments 1-156;
      receive first sono-enhanced ultrasound waves from the subject, wherein the first sono-enhanced ultrasound waves comprise the first emitted ultrasound waves enhanced via the sono-enhancing contrast agent;
      determine a first plurality of one or more neural data structures, using one or more processors, based on the first received sono-enhanced ultrasound waves;
      decode the first plurality into a first high-dimensional goal manifold, via a predictive machine learning algorithm;
      determine first instructions based on the first high-dimensional goal manifold, via an instruction-determining machine learning algorithm; and
      modulate the first ongoing psychophysical state of the subject, based on the first instructions.
273. The system of embodiment 271, comprising further instructions, that when executed by the one or more processors, cause the system to:
   emit second ultrasound waves to the subject, from the ultrasound transducer;
   receive second sono-enhanced ultrasound waves from the subject, wherein the second sono-enhanced ultrasound waves comprise the second emitted ultrasound waves enhanced via the sono-enhancing contrast agent;
   determine a second plurality of one or more neural data structures, using one or more processors, based on the second received sono-enhanced ultrasound waves;
   decode the second plurality or the first plurality into a second high-dimensional goal manifold, via a predictive machine learning algorithm;
   determine second instructions based on the first high-dimensional goal manifold, via an instruction-determining machine learning algorithm; and
   modulate the second ongoing psychophysical state of the subject, based on the second instructions.
274. A non-transitory computer-readable storage medium storing one or more programs, the one or more programs comprising instructions, which when executed by one or more processors of a system, cause the system to:
   emit first ultrasound waves to the subject, from an ultrasound transducer corresponding to the ultrasound transducer of any of embodiments 1-156;
   receive first sono-enhanced ultrasound waves from the subject, wherein the first sono-enhanced ultrasound waves comprise the first emitted ultrasound waves enhanced via the sono-enhancing contrast agent;
   determine a first plurality of one or more neural data structures, using one or more processors, based on the first received sono-enhanced ultrasound waves;
   decode the first plurality into a first high-dimensional goal manifold, via a predictive machine learning algorithm;
   determine first instructions based on the first high-dimensional goal manifold, via an instruction-determining machine learning algorithm; and
   modulate the first ongoing psychophysical state of the subject, based on the first instructions.
275. The non-transitory computer-readable storage medium of embodiment 274, comprising further instructions, that when executed by the one or more processors, cause the system to:
   emit second ultrasound waves to the subject, from the ultrasound transducer;
   receive second sono-enhanced ultrasound waves from the subject, wherein the second sono-enhanced ultrasound waves comprise the second emitted ultrasound waves enhanced via the sono-enhancing contrast agent;
   determine a second plurality of one or more neural data structures, using one or more processors, based on the second received sono-enhanced ultrasound waves;
   decode the second plurality or the first plurality into a second high-dimensional goal manifold, via a predictive machine learning algorithm;
   determine second instructions based on the first high-dimensional goal manifold, via an instruction-determining machine learning algorithm; and
   modulate the second ongoing psychophysical state of the subject, based on the second instructions.
276. A system comprising:
   one or more processors; and
   a memory communicatively coupled to the one or more processors and configured to store instructions that, when executed by the one or more processors, cause the system to:
      emit first ultrasound waves to the subject, from an ultrasound transducer corresponding to the ultrasound transducer of any of embodiments 1-156, to the one or more portions of the nervous system;
      receive first sono-enhanced ultrasound waves from the subject, wherein the first sono-enhanced ultrasound waves comprise the first emitted ultrasound waves enhanced via the sono-enhancing contrast agent;
      determine a first plurality of one or more neural data structures, using one or more processors, based on the first received sono-enhanced ultrasound waves;
      decode the first plurality, via an instruction-determining machine learning algorithm, into first instructions; and
      modulate the first ongoing psychophysical state of the subject, based on the first instructions.
277. The system of embodiment 276, comprising further instructions, that when executed by the one or more processors, cause the system to:
   emit second ultrasound waves to the subject, from an ultrasound transducer corresponding to the ultrasound transducer of any of embodiments 1-156, to the one or more portions of the nervous system;
   receive second sono-enhanced ultrasound waves from the subject, wherein the second sono-enhanced ultrasound waves comprise the second emitted ultrasound waves enhanced via the sono-enhancing contrast agent;
   determine a second plurality of one or more neural data structures, using one or more processors, based on the second received sono-enhanced ultrasound waves;
   decode the second plurality, via an instruction-determining machine learning algorithm, into second instructions; and
   modulate the second ongoing psychophysical state of the subject, based on the second instructions.
278. A non-transitory computer-readable storage medium storing one or more programs, the one or more programs comprising instructions, which when executed by one or more processors of a system, cause the system to:
   emit first ultrasound waves to the subject, from an ultrasound transducer corresponding to the ultrasound transducer of any of embodiments 1-156, to the one or more portions of the nervous system;
   receive first sono-enhanced ultrasound waves from the subject, wherein the first sono-enhanced ultrasound waves comprise the first emitted ultrasound waves enhanced via the sono-enhancing contrast agent;
   determine a first plurality of one or more neural data structures, using one or more processors, based on the first received sono-enhanced ultrasound waves;
   decode the first plurality, via an instruction-determining machine learning algorithm, into first instructions; and
   modulate the first ongoing psychophysical state of the subject, based on the first instructions.
279. The non-transitory computer-readable storage medium of embodiment 278, comprising further instructions, that when executed by the one or more processors, cause the system to:
   emit second ultrasound waves to the subject, from an ultrasound transducer corresponding to the ultrasound transducer of any of embodiments 1-156, to the one or more portions of the nervous system;
   receive second sono-enhanced ultrasound waves from the subject, wherein the second sono-enhanced ultrasound waves comprise the second emitted ultrasound waves enhanced via the sono-enhancing contrast agent;
   determine a second plurality of one or more neural data structures, using one or more processors, based on the second received sono-enhanced ultrasound waves;
   decode the second plurality, via an instruction-determining machine learning algorithm, into second instructions; and
   modulate the second ongoing psychophysical state of the subject, based on the second instructions.
280. A method of decoding latent neural states directly from one or more neural data structures, comprising:
   determining, using one or more processors, the one or more target latent neural states;
   receiving first ultrasound waves reflected from one or more portions of a nervous system from a subject, at the ultrasound transducer corresponding to any of embodiments 1-156;
   determining one or more first neural data structures from the ultrasound transducer; and
   decoding the one or more first neural data structures, via a regressor, using the one or more processors, into the one or more target latent neural states, thereby bypassing a beamforming transformation of the one or more neural data structures.
281. The method of embodiment 280, further comprising:
   determining first instructions, using one or more processors, based on the one or more target latent neural states;
   modulating the one or more portions of the nervous system of the subject, based on the first instructions;
   receiving second ultrasound waves reflected from the one or more portions of the nervous system from the subject, at the ultrasound transducer;
   determining one or more second neural data structures from the ultrasound transducer; and
   decoding the one or more second neural data structures, via the regressor, using the one or more processors, into the one or more target latent neural states, thereby bypassing the beamforming transformation of the one or more neural data structures.
282. A method of decoding latent neural states from one or more neural data structures, comprising:
   determining, using one or more processors, the one or more target latent neural states;
   receiving first ultrasound waves reflected from one or more portions of a nervous system from a subject, at the ultrasound transducer corresponding to any of embodiments 1-156;
   determining one or more first neural data structures from the ultrasound transducer;
   transforming the one or more first neural data structures into a first non-beamformed intermediate representation; and
   decoding the first non-beamformed intermediate representation, via a regressor, using the one or more processors, into the one or more target latent neural states, thereby bypassing a beamforming transformation of the first non-beamformed intermediate representation.
283. The method of embodiment 282, further comprising:
   determining first instructions, using one or more processors, based on the one or more target latent neural states;
   modulating the one or more portions of the nervous system of the subject, based on the first instructions;
   receiving second ultrasound waves reflected from the one or more portions of the nervous system from the subject, at the ultrasound transducer;
   determining one or more second neural data structures from the ultrasound transducer;
   transforming the one or more second neural data structures into a second non-beamformed intermediate representation; and
   decoding the second non-beamformed intermediate representation, via a regressor, using the one or more processors, into the one or more target latent neural states, thereby bypassing a beamforming transformation of the second non-beamformed intermediate representation.
284. The method of embodiment 283, wherein the first non-beamformed intermediate representation or the second non-beamformed intermediate representation comprises a wavelet decomposed representation, a time-frequency representation, or a dimensionality reduced representation.
285. The method of embodiment 284, wherein the time-frequency representation comprises a short-time Fourier transform.
286. The method of any of embodiments 283-285, wherein the first non-beamformed intermediate representation or the second non-beamformed intermediate representation comprises a registered representation.
287. The method of embodiment 286, wherein the registered representation comprises a motion compensated representation, a spatiotemporal registered representation, or an adaptively sampled representation.
288. The method of any of embodiments 284-287, wherein the modulating the one or more portions of the nervous system comprises emitting additional ultrasound waves to the subject, from the ultrasound transducer.
289. The method of any of embodiments 284-288, wherein the modulating the neural activity of the subject comprises electro-stimulating the one or more portions of the nervous system of the subject via one or more electrodes.
290. The method of any of embodiments 285-289, wherein the electro-stimulating comprises deep brain stimulation, transcranial magnetic stimulation (TMS), repetitive TMS, vagus nerve stimulation, transcranial direct current stimulation, or electrocorticography.
291. The method of any of embodiments 280-290, wherein the regressor comprises a linear regressor.
292. The method of embodiment 291, wherein the linear regressor comprises an ordinary least squares regressor, a ridge regressor, a lasso regressor, an elastic net regressor, a partial least squares regressor, a principal component regressor, a weighted least squares regressor, or a least angle regressor.
293. The method of any of embodiments 280-292, wherein the regressor comprises a non-linear regressor.
294. The method of embodiment 293, wherein the non-linear regressor comprises a polynomial regressor, a decision tree regressor, a random forest regressor, a logistic regressor, a support vector regressor with non-linear kernels, a neural network, a Gaussian process, a k-nearest neighbor regressor, a spline regressor, kernel ridge regressor, a generalized additive model, a multivariate adaptive regression spline regressor, a kernel regressor, a local regressor, a radial basis function network, an isotonic regressor, a projection pursuit regressor, a quantile regression forest, a sequential state estimator, a Kalman filter, or a multi-task learning regressor.
295. The method of any of embodiments 280-294, wherein the one or more neural data structures comprises radiofrequency data, in-phase or quadrature (IQ) data, or an envelope of the radiofrequency data.
296. The method of any of embodiments 280-295, wherein the one or more portions of the nervous system from the subject comprises a whole brain, a brain region, a neural circuit, or a single neuron.
297. The method of any of embodiments 280-296, wherein the one or more portions of the nervous system from the subject comprise a dorsolateral prefrontal cortex, frontopolar cortex, anterior cingulate cortex, inferotemporal cortex, visual cortex, auditory cortex, somatosensory cortex, posterior parietal cortex, temporoparietal junction, nucleus accumbens, ventral tegmental area, basal ganglia, orbitofrontal cortex, amygdala, insula, ventromedial prefrontal cortex, hippocampus, entorhinal cortex, parahippcampal gyrus, locus coerulus, reticular formation, ventral striatum, anterior insula, Broca's area, Wernicke's area, medial prefrontal cortex, temporoparietal junction, anterior insula, posterior insula, supplementary motor area, pre-supplementary motor area, or a combination thereof.
298. The method of any of embodiments 280-297, wherein the ultrasound transducer comprises an ultrasonic transducer array.
299. The method of embodiment 298, wherein the ultrasonic transducer array is a capacitive micromachined ultrasonic transducer (CMUT) array or a piezoelectric micromachined ultrasonic transducer (PMUT) array.
300. The method of embodiment 299, wherein the CMUT array comprises a flat membrane CMUT, a curved membrane CMUT, a dual-electrode CMUT, a collapsed-mode CMUT, a wafer-bonded CMUT, a surface-micromachined CMUT, a CMUT-on-CMOS integrated device, a piston-shaped CMUT, an annular array CMUT, or a flexible CMUT.
301. The method of embodiment 299, wherein the PMUT array comprises a unimorph PMUT, a bimorph PMUT, a dome-shaped PMUT, a suspended membrane PMUT, an aluminum nitride PMUT, a lead zirconate titanate PMUT, a scandium-doped aluminum nitride PMUT, a lithium niobate PMUT, a polyvinylidene fluoride PMUT, a thin-film PMUT, or a flexible PMUT.
302. The method of any of embodiments 280-301, wherein the ultrasound transducer is an implantable ultrasound transducer.
303. The method of any of embodiments 280-302, wherein the ultrasound transducer is a non-implantable ultrasound transducer.
304. The method of embodiment 303, wherein the non-implantable ultrasound transducer comprises a wearable ultrasound transducer.
305. The method of any of embodiments 280-304, wherein the subject comprises a sono-enhancing contrast agent.
306. The method of embodiment 305, wherein the sono-enhancing contrast agent comprises a biological sono-enhancing contrast agent, a polymeric sono-enhancing contrast agent, or a pharmaceutical sono-enhancing contrast agent.
307. The method of embodiment 306, wherein the biological sono-enhancing contrast agent comprises a genetically-encoded sono-enhancing contrast agent.
308. The method of embodiment 307, wherein the genetically-encoded sono-enhancing contrast agent comprises an ultrasonic reporter of calcium, an ultrasonic reporter of voltage, or an ultrasonic reporter of a neurotransmitter.
309. The method of any of embodiments 280-308, wherein the subject comprises a human subject or a non-human primate subject.
310. The method of any of embodiments 280-309, wherein the subject has, or is suspected of having a neural dysfunction.
311. The method of embodiment 310, wherein the neural dysfunction comprises a motor dysfunction, a sensory dysfunction, a cognitive dysfunction, a neurological condition, or a neuropsychiatric dysfunction.
312. The method of embodiment 311, wherein the motor dysfunction comprises a spinal cord injury, amyotrophic lateral sclerosis, locked-in syndrome, stroke, Parkinson's disease, or essential tremor.
313. The method of embodiment 311 or 312 wherein the sensory dysfunction comprises blindness, deafness, epilepsy, a loss of chemosensation, or a loss of somatosensation.
314. The method of any of embodiments 311-313, wherein the neuropsychiatric dysfunction comprises clinical depression, schizophrenia, addiction, post-traumatic stress disorder, eating disorder, or sleep disorder.
315. A system comprising:
   one or more processors; and
   a memory communicatively coupled to the one or more processors and configured to store instructions that, when executed by the one or more processors, cause the system to:
      determine, using one or more processors, the one or more target latent neural states;
      receive first ultrasound waves reflected from one or more portions of a nervous system from a subject, at the ultrasound transducer corresponding to any of embodiments 1-156;
      determine one or more first neural data structures from the ultrasound transducer; and
      decode the one or more first neural data structures, via a regressor, using the one or more processors, into the one or more target latent neural states, thereby bypassing a beamforming transformation of the one or more neural data structures.
316. The system of embodiment 315, comprising further instructions, that when executed by the one or more processors, cause the system to:
   determine first instructions, using one or more processors, based on the one or more target latent neural states;
   modulate the one or more portions of the nervous system of the subject, based on the first instructions;
   receive second ultrasound waves reflected from the one or more portions of the nervous system from the subject, at the ultrasound transducer;
   determine one or more second neural data structures from the ultrasound transducer; and
   decode the one or more second neural data structures, via the regressor, using the one or more processors, into the one or more target latent neural states, thereby bypassing the beamforming transformation of the one or more neural data structures.
317. A system comprising:
   one or more processors; and
   a memory communicatively coupled to the one or more processors and configured to store instructions that, when executed by the one or more processors, cause the system to:
      determine, using one or more processors, the one or more target latent neural states;
      receive first ultrasound waves reflected from one or more portions of a nervous system from a subject, at the ultrasound transducer corresponding to any of embodiments 1-156;
      determine one or more first neural data structures from the ultrasound transducer;
      transform the one or more first neural data structures into a first non-beamformed intermediate representation; and
      decode the first non-beamformed intermediate representation, via a regressor, using the one or more processors, into the one or more target latent neural states, thereby bypassing a beamforming transformation of the first non-beamformed intermediate representation.
318. The system of embodiment 317, comprising further instructions, that when executed by the one or more processors, cause the system to:
   determine first instructions, using one or more processors, based on the one or more target latent neural states;
   modulate the one or more portions of the nervous system of the subject, based on the first instructions;
   receive second ultrasound waves reflected from the one or more portions of the nervous system from the subject, at the ultrasound transducer;
   determine one or more second neural data structures from the ultrasound transducer;
   transform the one or more second neural data structures into a second non-beamformed intermediate representation; and
   decode the second non-beamformed intermediate representation, via a regressor, using the one or more processors, into the one or more target latent neural states, thereby bypassing a beamforming transformation of the second non-beamformed intermediate representation.
319. A non-transitory computer-readable storage medium storing one or more programs, the one or more programs comprising instructions, which when executed by one or more processors of a system, cause the system to:
   determine, using one or more processors, the one or more target latent neural states;
   receive first ultrasound waves reflected from one or more portions of a nervous system from a subject, at the ultrasound transducer corresponding to any of embodiments 1-156;
   determine one or more first neural data structures from the ultrasound transducer; and
   decode the one or more first neural data structures, via a regressor, using the one or more processors, into the one or more target latent neural states, thereby bypassing a beamforming transformation of the one or more neural data structures.
320. The non-transitory computer-readable storage medium of embodiment 319, comprising further instructions, that when executed by the one or more processors, cause the system to:
   determine first instructions, using one or more processors, based on the one or more target latent neural states;
   modulate the one or more portions of the nervous system of the subject, based on the first instructions;
   receive second ultrasound waves reflected from the one or more portions of the nervous system from the subject, at the ultrasound transducer;
   determine one or more second neural data structures from the ultrasound transducer; and
   decode the one or more second neural data structures, via the regressor, using the one or more processors, into the one or more target latent neural states, thereby bypassing the beamforming transformation of the one or more neural data structures.
321. A non-transitory computer-readable storage medium storing one or more programs, the one or more programs comprising instructions, which when executed by one or more processors of a system, cause the system to:
   determine, using one or more processors, the one or more target latent neural states;
   receive first ultrasound waves reflected from one or more portions of a nervous system from a subject, at the ultrasound transducer corresponding to any of embodiments 1-156;
   determine one or more first neural data structures from the ultrasound transducer;
   transform the one or more first neural data structures into a first non-beamformed intermediate representation; and
   decode the first non-beamformed intermediate representation, via a regressor, using the one or more processors, into the one or more target latent neural states, thereby bypassing a beamforming transformation of the first non-beamformed intermediate representation.
322. The non-transitory computer-readable storage medium of embodiment 321, comprising further instructions, that when executed by the one or more processors, cause the system to:
   determine first instructions, using one or more processors, based on the one or more target latent neural states;
   modulate the one or more portions of the nervous system of the subject, based on the first instructions;
   receive second ultrasound waves reflected from the one or more portions of the nervous system from the subject, at the ultrasound transducer;
   determine one or more second neural data structures from the ultrasound transducer;
   transform the one or more second neural data structures into a second non-beamformed intermediate representation; and
   decode the second non-beamformed intermediate representation, via a regressor, using the one or more processors, into the one or more target latent neural states, thereby bypassing a beamforming transformation of the second non-beamformed intermediate representation.

### EXAMPLES

Examples 1-3 provide diagrams illustrating the operation of the ultrasound transducers (e.g., ultrasound transducer devices) for nervous systems comprising a sono-enhancing contrast agent, and their use in the methods and systems described herein. Example 1 provides diagrams describing the scaling parameters of the ultrasound transducers, across the different neural scales. Example 2 provides diagrams describing the operation of one or more sono-enhancing contrast agents for use with the one or more ultrasound transducers. Example 3 provides diagrams describing the implementation of the different scale-optimized ultrasound transducers, in conjunction with the nervous system of a subject comprising the sono-enhancing contrast agent.

### Example 1. Scaling Parameters of the Ultrasound Transducers

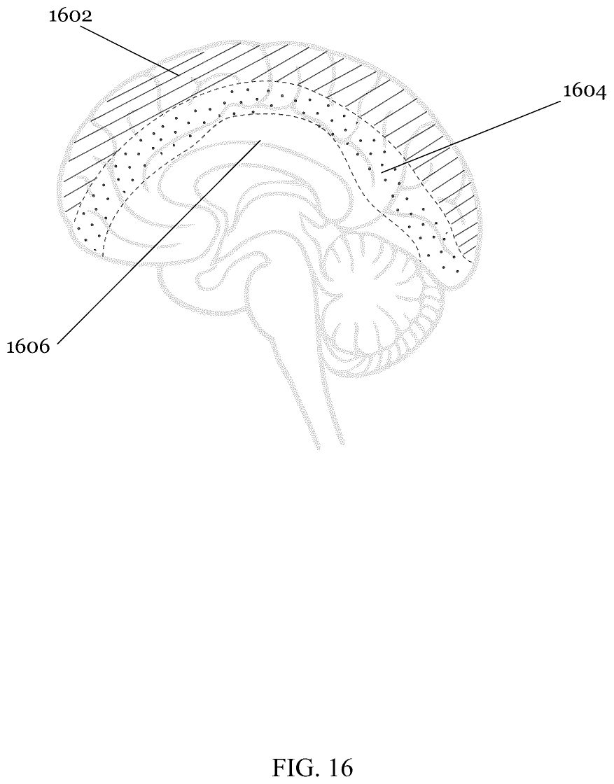

The ultrasound transducers described in the present examples are optimized for a given neural scale, e.g., they are scale-optimized ultrasound transducers. FIG. 16 provides a diagram illustrating example brain depths that can be accessed via different parameters for the ultrasound transducers, e.g., varying element widths of the ultrasound transducer arrays, or varying frequencies. Various depths of a brain, relative to the brain's surface, are depicted, at depth 1602 of 0.5 cm, at depth 1604 of 2.0 cm, and at depth 1606 of 4.0 cm. Each depth of the brain can correspond to a maximum monitoring (e.g., imaging) depth or modulating depth of the ultrasound transducer, depending on the scale to which the ultrasound transducer is optimized. At each depth, a corresponding approximate frequency and minimum optical resolution are also depicted. For example, at a depth of 0.5 cm, a minimum optical resolution of 25 microns and a frequency of 60 MHz is depicted; at a depth of 2.0 cm, a minimum optical resolution of 100 microns and a frequency of 15 MHz is depicted; at a depth of 4.0 cm, a minimum optical resolution of 200 microns and a frequency of 7.5 MHz is depicted; and at a depth of 6.0 cm, a minimum optical resolution of 300 microns and a frequency of 5.0 MHz is depicted. For each depth, the optical resolution can be further improved, e.g., decreased, if a superresolution method, such as a computational superresolution method, is used. For example, ultrasound images acquired across multiple angles at approximately similar timepoints can be analyzed to infer one or more super-resolved images. In addition, the use of common imaging landmarks can be leveraged to infer or generate a super-resolved image, and/or one or more machine learning-based methods (e.g., a transformer, a convolutional neural network, a generative adversarial network, or a deep unfolding network) can be used to infer or generate a super-resolved image.

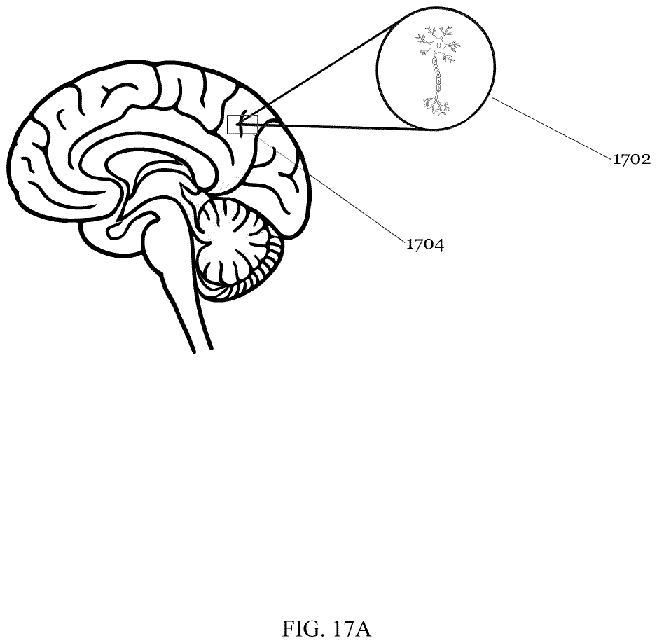

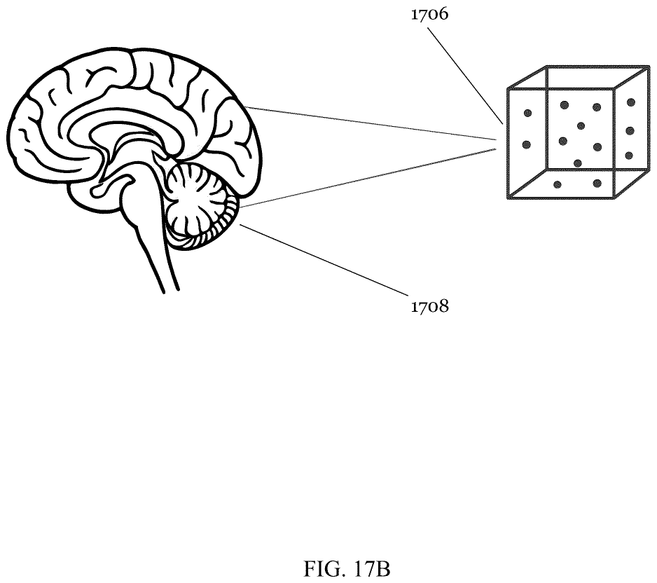

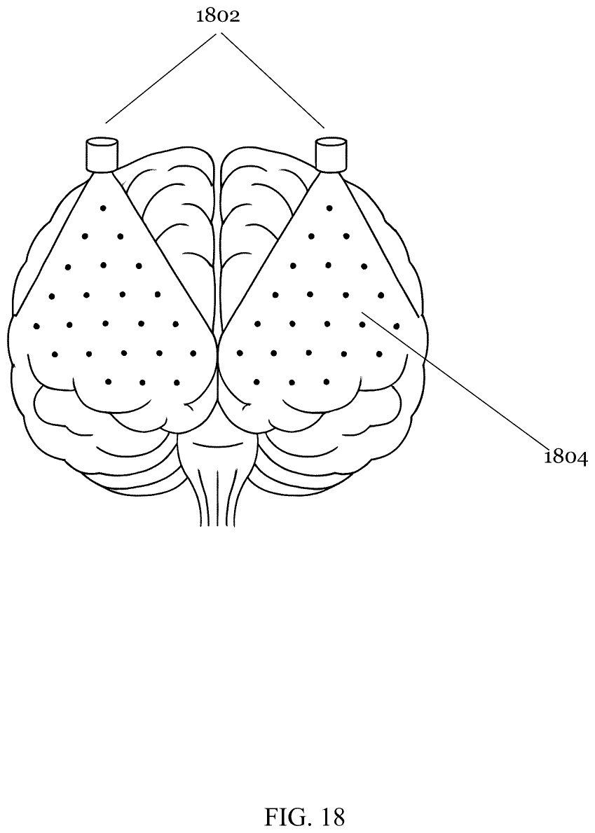

FIG. 17A provides a diagram illustrating the imaging or modulating parameters for an ultrasound transducer optimized for operating at a single-neuron scale. A voxel 1702 corresponding to the monitoring or modulating resolution of the single-neuron-optimized ultrasound transducer is depicted at an exemplary resolution of, for example, 25 microns—that is, as a cubic voxel comprising a unit length of 25 microns. The 25 micron resolution can correspond to an ultrasound frequency of 60 MHz, as emitted by the ultrasound transducer. The 25 micron resolution can correspond to a more limited field of view 1704, e.g., imaging or modulating volume, of e.g., 1×1×0.3 cm. At these imaging or modulating parameters, a single neuron can be resolved. A complete single neuron may not be resolved within a given voxel or even within a single field of view, however, depending on the single neuron's geometry. That is, some neurons may possess certain geometries, e.g., long axonal projections, that may exceed the dimensions of the imaging or modulating volume. The resolution and corresponding field of view provided by the single-neuron-optimized ultrasound transducer should be understood as being capable of, but not necessitating, resolving neural structures at the level of the single neuron.

FIG. 17B provides a diagram illustrating the imaging or modulating parameters for an ultrasound transducer optimized for operating at a brain-wide scale. A voxel 1706 corresponding to the monitoring or modulating resolution of the brain-wide-optimized ultrasound transducer is depicted at an exemplary resolution of 300 microns—that is, as a cubic voxel comprising a unit length of 300 microns. The 300 micron resolution can correspond to an ultrasound frequency of 5 MHz, as emitted by the ultrasound transducer. The 300 micron resolution can correspond to a less limited field of view 1708, e.g., imaging or modulating volume, of approximately or near approximately the whole brain. At these imaging or modulating parameters, a circuit node, e.g., a population of approximately 1000 neurons, can be resolved per voxel, as indicated in voxel 1706. Each dot in voxel 1706 represents a neuron—the optical resolution of voxel 1706 cannot resolve neurons to the same detailed resolution as voxel 1702. Accordingly, the imaging or the modulating of the whole brain can be performed, at the resolution of approximately a circuit node, e.g., neural circuit.

### Example 2. Scale-Optimized Ultrasound Transducers and Sono-Enhancing Contrast Agents

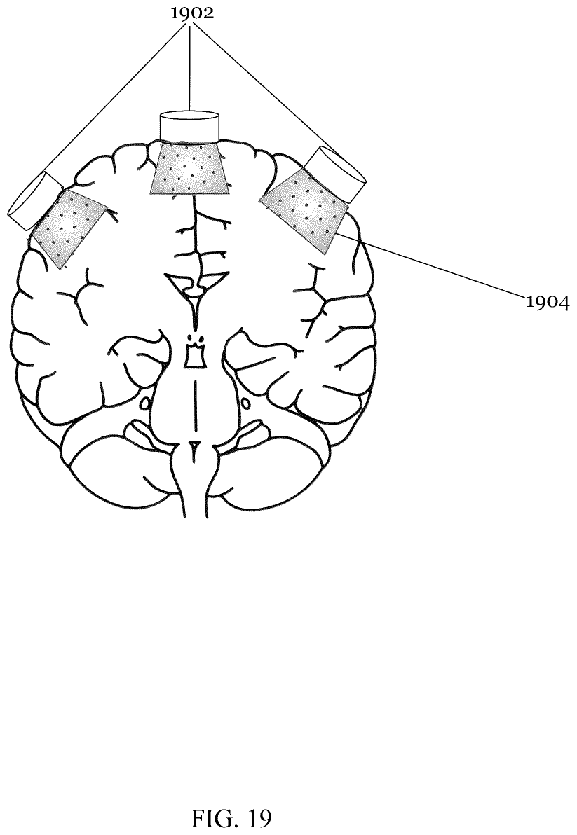

The present example describes configurations for implementing scale-optimized ultrasound transducers and their associated sono-enhancing contrast agents, for various neural scales. FIG. 18 provides a diagram illustrating the use of the ultrasound transducers optimized for brain-wide monitoring or modulating of a nervous system comprising sono-enhancing contrast agents. Ultrasound transducers 1802 emit ultrasound waves at fields of view optimized for brain-wide monitoring or modulating. The ultrasound waves interact with neurons possessing sono-enhancing contrast agents, such as neuron 1804. The combination of both the brain wide-optimized ultrasound transducers 1802 and the sono-enhancing contrast agent-expressing neurons 1804 result in the monitoring or modulating of a subject's nervous system at a brain wide scale, at a high signal-to-noise ratio. Such a scale can include approximately 1 million neurons, or an imaging/modulating volume of 700 cm³, at a resolution of approximately 300 microns, per field of view. In the example depicted in FIG. 19, multiple fields of view from the ultrasound transducers are employed, such that the fields of view are overlapping. In addition, other neurons expressing sono-enhancing contrast agents are shown as dots in FIG. 18. The neurons expressing the sono-enhancing contrast agents need not be confined to the positions depicted in FIG. 18. The sono-enhancing contrast agent-expressing neurons depicted in FIG. 18 show a sample of neurons that exist within the field of views of the ultrasound transducers.

FIG. 19 provides a diagram illustrating the use of the ultrasound transducers optimized for monitoring or modulating brain regions or neural circuits, for a nervous system comprising sono-enhancing contrast agents. Ultrasound transducers 1902 emit ultrasound waves at fields of view optimized for brain region-level or neural circuit-level monitoring or modulating. The ultrasound waves interact with neurons possessing sono-enhancing contrast agents, such as neuron 1904. The ultrasound waves interact with neurons possessing sono-enhancing contrast agents, such as neuron 1904. The combination of both the brain region-optimized or neural circuit-optimized ultrasound transducers 1902 and the sono-enhancing contrast agent-expressing neurons 1904 result in the monitoring or modulating of a subject's nervous system at a mesoscale, at a high signal-to-noise ratio. Such a scale can include approximately 1 million neurons, or an imaging/modulating volume of 12 cm³, at a resolution of approximately 100 microns, per field of view. In the example depicted in FIG. 19, multiple fields of view from the ultrasound transducers are employed, such that the fields of view are not necessarily overlapping. In addition, other neurons expressing sono-enhancing contrast agents are shown as dots in FIG. 19. The neurons expressing the sono-enhancing contrast agents need not be confined to the positions depicted in FIG. 19. The sono-enhancing contrast agent-expressing neurons depicted in FIG. 19 show a sample of neurons that exist within the field of views of the ultrasound transducers.

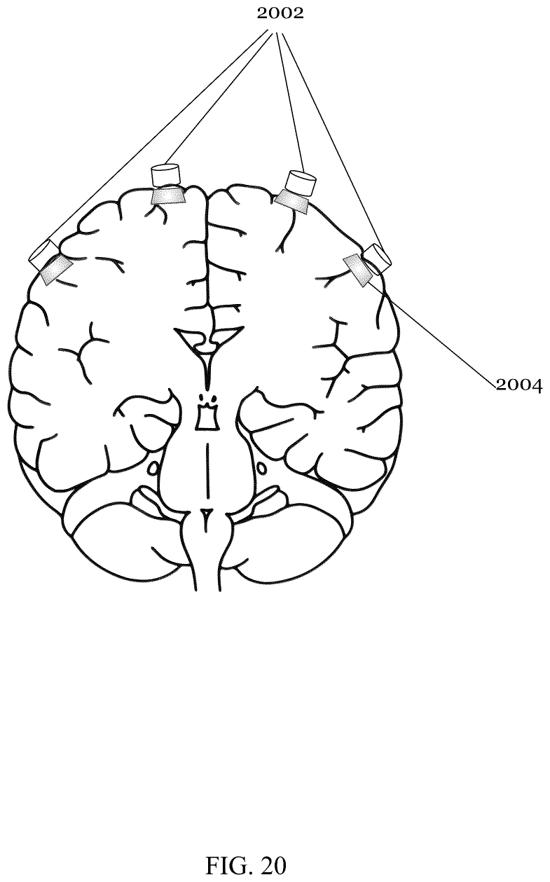

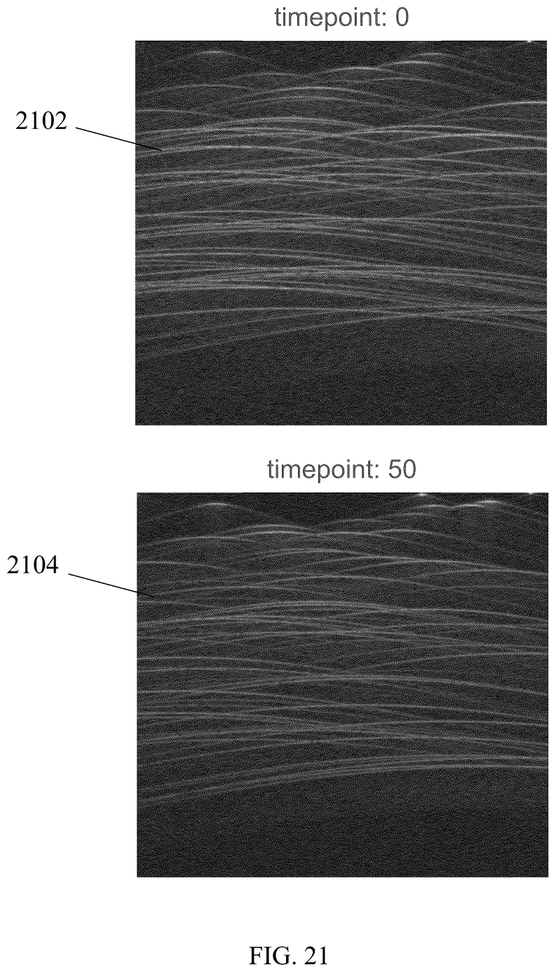

FIG. 20 provides a diagram illustrating the use of the ultrasound transducers optimized for monitoring or modulating neural circuits or single neurons for a nervous system comprising sono-enhancing contrast agents. Ultrasound transducers 2002 emit ultrasound waves at fields of view optimized for neural circuit-level or single neuron-level monitoring or modulating. The ultrasound waves interact with neurons possessing sono-enhancing contrast agents, such as neuron 2004. The ultrasound waves interact with neurons possessing sono-enhancing contrast agents, such as neuron 2004. The combination of both the neural circuit-optimized or single neuron-optimized ultrasound transducers 2002 and the sono-enhancing contrast agent-expressing neurons 2004 result in the monitoring or modulating of a subject's nervous system at a microscale, at a high signal-to-noise ratio. Such a scale can include approximately 1 million neurons, or an imaging/modulating volume of 0.6 cm³, at a resolution of approximately 25 microns, per field of view. In the example depicted in FIG. 20, multiple fields of view from the ultrasound transducers are employed, such that the fields of view are not necessarily overlapping. In addition, other neurons expressing sono-enhancing contrast agents are shown as dots in FIG. 20. The neurons expressing the sono-enhancing contrast agents need not be confined to the positions depicted in FIG. 20. The sono-enhancing contrast agent-expressing neurons depicted in FIG. 20 show a sample of neurons that exist within the field of views of the ultrasound transducers.

### Example 3. Point Scatter Simulation Data for Validating Target Latent Neural States

The present example provides point scatter simulation data for validating the decoding of target latent neural states for a subject. M time-varying latent neural states that represent underlying neural activity patterns were first defined. The M time-varying latent states were then decoded directly from simulated radiofrequency signals. That is, N point scatterers were placed at arbitrary Cartesian coordinates in 2D space. The N point scatterers were used to approximate acoustic scatters with reflectances that correlate with underlying neural activity. Therefore, the N point scatterers were used to simulate a sono-enhancing contrast agent, such as ultrasound reporters of calcium, because ultrasound reporters of calcium can effectively behave as neural activity-dependent point sources for ultrasound waves. Each point scatterer in the N point scatterers were then assigned an acoustic impedance contrast value, based on the defined M time-varying latent neural states. From the acoustic impedance contrast values, radiofrequency signals were derived. In the simulation results, the derived radiofrequency signals corresponded to signals from a linear array of ultrasound transducer elements from an ultrasonic transducer array. Image 2102 shows 2D ultrasound wave patterns from the N point scatterers at timepoint 0, and image 2104 shows 2D ultrasound wave patterns from N point scatterers at timepoint 50, where each timepoint represents an arbitrary unit of time under the simulation conditions. A key distinction between image 2102 at timepoint 0 and image 2104 at timepoint 50, however, is the differing magnitudes of backscattered echoes corresponding to each point of the N point scatterers. The differing magnitudes of the backscattered echoes are the result of changes in a neural state for the simulated subject. That is, changes in the neural state results in correlated changes across the N point scatterers. These correlated changes across the N point scatterers are represented, e.g., realized, by a target latent neural state, via random projections to each of the points in the N point scatterers. The target latent neural state can be recovered directly from the simulated point scatter data, as shown, for example, in image 2102 and image 2104.

It should be understood from the foregoing that, while particular implementations of the disclosed methods and systems have been illustrated and described, various modifications can be made thereto and are contemplated herein. It is also not intended that the disclosure be limited by the specific examples provided within the specification. While the disclosure has been described with reference to the aforementioned specification, the descriptions and illustrations of the preferable embodiments herein are not meant to be construed in a limiting sense. Furthermore, it shall be understood that all aspects of the disclosure are not limited to the specific depictions, configurations or relative proportions set forth herein which depend upon a variety of conditions and variables. Various modifications in form and detail of the embodiments of the disclosure may be apparent to a person skilled in the art. It is therefore contemplated that the disclosure shall also cover any such modifications, variations, and equivalents.

1. A method of modulating or monitoring a subject, the method comprising:
emitting, from an ultrasound transducer, ultrasound waves to the subject;
sono-enhancing the emitted ultrasound waves, via a sono-enhancing contrast agent in the subject, to form sono-enhanced ultrasound waves;
receiving, by the ultrasound transducer, the sono-enhanced ultrasound waves from the subject; and
determining, using one or more processors, one or more neural data structures based on the received sono-enhanced ultrasound waves.

2. The method of claim 1, wherein modulating or monitoring of the subject comprises modulating or monitoring a brain region, a neural circuit, or a single neuron of the subject.

3. The method of claim 1, wherein the one or more neural data structures correspond to an imaging volume of at most 0.2 cm³, or 0.6 cm³, or comprise imaging data at an imaging depth of at least 0.04 cm, 0.1 cm, or 0.4 cm.

4. The method of claim 1, wherein the one or more neural data structures comprise imaging data at an optical resolution of at least 20 microns, 25 microns, or 50 microns.

5. The method of claim 1, wherein the ultrasound waves have a frequency of at most 100 MHz, 60 MHz, or 15 MHz.

6. The method of claim 1, wherein the one or more neural data structures comprise precursor imaging data at a computational superresolution.

7. The method of claim 6, wherein the computational superresolution corresponds to a spatial resolution less than a width of an element in an ultrasonic transducer array of the ultrasound transducer.

8. The method of claim 1, wherein the one or more neural data structures comprise hemodynamic data, neural activity data, or neural anatomy data.

9. The method of claim 1, wherein the ultrasound transducer comprises an ultrasonic transducer array having elements, the elements having a shape that is circular, elliptical, square, rectangular, hexagonal, regular, or irregular.

10. The method of claim 9, wherein the ultrasonic transducer array is a capacitive micromachined ultrasonic transducer (CMUT) array or a piezoelectric micromachined ultrasonic transducer (PMUT) array.

11. The method of claim 1, wherein the sono-enhancing contrast agent comprises a biological sono-enhancing contrast agent, a polymeric sono-enhancing contrast agent, or a pharmaceutical sono-enhancing contrast agent.

12. The method of claim 11, wherein the biological sono-enhancing contrast agent comprises a genetically-encoded sono-enhancing contrast agent that is an ultrasonic reporter of calcium, voltage, or a neurotransmitter.

13. The method of claim 1, wherein the ultrasound transducer provides neural augmentation to the subject.

14. The method of claims 13, wherein the neural augmentation comprises sensorimotor integration, sensory augmentation, integration with virtual reality, integration with augmented reality, modulation of a brain state, or integration with artificial intelligence.

15. A system comprising:
one or more processors; and
a memory communicatively coupled to the one or more processors and configured to store instructions that, when executed by the one or more processors, cause the system to:
emit, using an ultrasound transducer, ultrasound waves to a brain of a subject;
sono-enhance the emitted ultrasound waves, via a sono-enhancing contrast agent in the subject, to form sono-enhanced ultrasound waves;
receive, by the ultrasound transducer, the sono-enhanced ultrasound waves from the subject; and
determine, using the one or more processors, one or more neural data structures based on the received sono-enhanced ultrasound waves.

16. The system of claim 15, wherein the ultrasound transducer comprises an ultrasonic transducer array including elements having a width of 100 to 500 microns.

17. The system of claim 15, wherein the one or more neural data structures correspond to an imaging volume of at most 100 cm³, 700 cm³, or 1000 cm³.

18. The system of claim 15, wherein the one or more neural data structures comprise imaging data at an optical resolution of at least 100 microns, 150 microns, 250 microns, 300 microns, or 500 microns.

19. The system of claim 15, wherein the one or more neural data structures comprise imaging data at an imaging depth of at least 4.0 cm, 6.0 cm, or 8.0 cm.

20. The system of claim 15, wherein the ultrasound waves have a frequency of at most 5.0 MHz, 2.5 MHz, or 1.0 MHz.
